(19) United States
(12) Patent Application Publication
(10) Pub. No.: US 2006/0127397 A1
(43) Pub. Date: Jun. 15, 2006

(54) RAG polypeptides, nucleic acids, and their use

(76) Inventor: Stephen S. Strittmatter, Guilford, CT (US)

Correspondence Address: FOLEY & LARDNER LLP, 111 HUNTINGTON AVENUE, 26TH FLOOR, BOSTON, MA, 02199-7610, US

(21) Appl. No.: 11/194,074

(22) Filed: Jul. 29, 2005

Related U.S. Application Data
(63) Continuation of application No. PCT/US04/02758, filed on Jan. 30, 2004.
(60) Provisional application No. 60/443,485, filed on Jan. 30, 2003.

(51) Int. Cl. A61K 48/00; A61K 38/17; A61K 39/395

(52) U.S. Cl. 424/143.1; 514/12; 514/44

(57) ABSTRACT

The present invention relates generally to regeneration associated genes (RAGs). More specifically, the invention relates to structure-based methods and compositions useful in designing, identifying, and producing molecules which act as functional modulators of RAGs and RAG polypeptides. The invention further relates to methods of detecting, preventing, and treating RAG-associated disorders.

### STATEMENT CONCERNING GOVERNMENT RIGHTS IN FEDERALLY SPONSORED RESEARCH

Research involved in developing this Invention was supported, in whole or In part, via Grant No. R01 NS39962 from the United States National Institutes of Health. The Government of the United States of America may have certain rights in this application.

### FIELD OF THE INVENTION

The present invention relates generally to regeneration-associated genes (RAGs). More specifically, the invention relates to structure-based methods and compositions useful in designing, identifying, and producing molecules which act as functional modulators of RAGs and RAG polypeptides. The invention further relates to methods of detecting, preventing, and treating RAG-associated disorders.

### BACKGROUND OF THE INVENTION

In adult mammals, neurons of the peripheral nervous system (PNS) can regenerate an axon even after complete nerve transection. In contrast, axonal regeneration is minimal in the central nervous system (CNS). The failure of neurons to regenerate in the damages brain and spinal cord is a major cause of human suffering in many common neurological disorders such as brain and spinal cord trauma, stroke, cerebral palsy and multiple sclerosis.

Several factors are likely to contribute to this marked and clinically important disparity in regeneration capacity of the PNS and CNS. The environment surrounding the injured nerve differs between the PNS and CNS, partially accounting for the differential response to axonal injury. In the CNS, oligodendrocytes and glial scar tissue block regeneration by expression of inhibitory molecules such as Nogo, myelin-associated glycoprotein, oligodendrocyte myelin glycoprotein, and chondroitin sulfate proteoglycans (McKerracher et al.,, 1994; Fawcett and Asher, 1999; Niederost et al., 1999; GrandPre et al., 2000, 2002; Fournier and Stritimatter, 2001; Wang et al., 2002). The distal degenerating stump of a peripheral nerve lacks these axon growth inhibitors and is a fertile environment for the growth of axons. indeed, CNS axons can extend long distances through transplanted peripheral nerve stumps or through a transplanted embryonic nervous system (Richardson et al., 1980; Coumans et al., 2001; Bregman et al., 2002).

Cell-autonomous factors also appear to specify the capacity for axonal regeneration, regardless of extracellular factors. Adult CNS neurons never extend axons as rapidly as do their embryonic or PNS counterparts (Goldberg et al., 2002). Peripheral nerve injury before central injury appears to induce a growth-competent state that allows a degree of CNS growth from the central process of the same neuron injured peripherally (Chong et al., 1999; Neumann and Woolf, 1999). This “conditioning” effect suggests that a program of gene expression uniquely induced by peripheral axotomy can support axon extension. Data from different paradigms indicate that signaling pathways mediated by CAMP (Qiu et al., 2002a,b) and Janus activated kinase (JAK)-signal transducer and activator of transcription (STAT) phosphorylatlon (Liu and Snider, 2001) may participate in changes in axonal regeneration competence.

Molecular determinants of axonal regeneration can be identified by characterizing gene expression profiles before and after peripheral nerve injury. Regeneration-associated genes (RAGs) have been identified using a range of methods, including differential-display PCR (Kiryu et al., 1995), expressed sequence tag analysis (Tanabe et al., 1999), differential screening of a subtractive cDNA library (Araki et al., 2001), subtractive hybridization (Costigan et al., 1998), and cDNA microarray analysis (Bonilla et al., 2002; Costigan et al., 2002). Known RAGs encode many classes of proteins, including but not limited to neurotrophin receptors (Funakoshi et al., 1993), cytoskeletal elements (Muma et al., 1990;

Moskowitz and Oblinger, 1995), neuropeptides (Villar et al., 1989; Wakisaka et al., 1991), transcription factors (Tsujino et al., 2000), and molecular chaperones (Costigan et al., 1998; Lewis et al., 1999; Benn et al., 2002). The observation that many RAGs are developmentally prevalent genes has led to the notion that “regeneration recapitulates development.” Growth-associated protein (GAP)-43 is perhaps the prototypical RAG and fits this paradigm (Skene and Willard, 1981; Skene, 1989). In a recent cDNA microarray study, we observed that the small proline rich protein 1A (SPRRLA) Is very strongly induced by peripheral axotomy and promotes axon growth (Bonilla et al., 2002); however, SPRRIA is not found in any developing neurons, so the development-regeneration link is not universal.

As a result of their biological activities, there remains a need for the development of RAG-based therapeutics for treating injured or diseased mammalian tissue, including, for example, therapeutic compositions for inducing regenerative healing of injured neurons, e.g., stroke, or inhibiting neuron growth, e.g., neuroblastoma, as well as therapeutic compositions for preserving or restoring healthy metabolic properties in diseased tissue, e.g., degenerative nerve disorders.

### SUMMARY OF THE INVENTION

The present invention relates generally to RAGs. More specifically, the invention relates to structure-based methods and compositions useful in designing, identifying, and producing molecules which act as functional modulators of RAGs and RAG polypeptides. The invention further relates to methods of detecting, preventing, and treating RAG-associated disorders.

In one aspect, the invention includes a method of regenerating neurons comprising administering to a subject in need thereof a RAG compound that modulates the activity of a polypeptide encoded by a polynucleotide selected from the group consisting of RAG ID NOs:1-281 in amounts effective to cause neuronal regeneration. RAG compounds can include polypeptides, peptides, polynucleoudes, anti-RAG antibodies and small molecules. Also included are variants, analogs homologs, or fragments of the polypeptide and polynucleofide sequences, and small molecules incorporating these. In a preferred embodiment of the method, the RAG compound modulates the activity of an Fn14 polypeptide (RAG ID NO:6) in amounts effective to cause neuronal regeneration. In another embodiment of the method, the 30 subject is a human. In another embodiment of the method, the RAG compound increases the activity of a polypeptide encoded by a polynucleotide selected from the group consisting of RAG ID NOs:1-281. In preferred embodiment of the method, the RAG compound increases the activity of an Fn14 polypeptide (RAG ID NO:6). In another embodiment of the method, the RAG compound Is an F14 agonist. In another embodiment of the method, the RAG compound is a TWEAK polypeptide, homolog, or fragment thereof. In another embodiment of the method, the RAG compound increases the expression of a polypeptide encoded by a polynucleotide selected from the group consisting of RAG ID NOs:1-281. In a preferred embodiment of the method, the RAG compound increases the expression of an expression of an Fn14 polypeptide (RAG ID NO:6). In another embodiment of the method, the RAG compound decreases the activity of a polypeptide encoded by a polynucdeotide selected from the group consisting of RAG ID NOs:1-281. In another embodiment of the method, the RAG compound decreases the activity of an Fn14 polypeptide (RAG ID NO:6). In another embodiment of the method, the RAG compound is an Fn14 antagonist In another embodiment, the RAG compound decreases the expression of a polypeptide encoded by a polynucleotide selected from the group consisting of RAG ID NOs:1-281. In a preferred embodiment, the RAG compound decreases the expression of an Fn14 polypeptide (RAG ID NO:6).

In another aspect, the invention includes method of inhibiting neuron growth comprising administering to a subject in need thereof a RAG compound that modulates the activity of a polypeptide encoded by a polynudeotide selected from the group consisting of RAG ID NOs:1-281 in amounts effective to cause inhibition of neuronal growth. RAG compounds can include polypeptides, peptides, polynucleotides, anti-RAG antibodies and small molecules. Also included are variants, analogs homologs, or fragments of the polypeptide and polynucleotide sequences, and small molecules incorporating these. In a preferred embodiment of the method, the RAG compound modulates the activity of an Fn14 polypeptide (RAG ID NO:6) in amounts effective to inhibit neuronal growth. In another embodiment of the method, the subject is a human. In another embodiment of the method, the RAG compound increases the activity of a polypeptide encoded by a polynucleotide selected from the group consisting of RAG ID NOs:1-281. In a preferred embodiment of the method, the RAG compound increases the activity of an Fn14 polypeptide (RAG ID NO:6). In another embodiment of the method, the RAG compound is an F14 agonist. In another embodiment of the method, the RAG compound is a TWEAK polypeptide, homolog, or fragment thereof. In another embodiment of the method, the RAG compound increases the expression of a polypeptide encoded by a polynucleotide selected from the group consisting of RAG ID NOs:1-281. In a preferred embodiment of the method, the RAG compound increases the expression of an expression of an Fn14 polypeptide (RAG ID NO:6). In another embodiment of the method, the RAG compound decreases the activity of a polypeptide encoded by a polynucleotide selected from the group consisting of RAG ID NOs:1-281. In another embodiment of the method, the RAG compound decreases the activity of an Fn14 polypeptide (RAG ID NO:6). In another embodiment of the method, the RAG compound is an Fn14 antagonist. In another embodiment, the RAG compound decreases the expression of a polypeptide encoded by a polynucleotide selected from the group consisting of RAG ID NOs:1-281. In a preferred embodiment, the RAG compound decreases the expression of an Fn14 polypeptide (RAG ID NO:6).

In another aspect, the invention includes a method of regenerating neurons comprising administering to a subject in need thereof an effective amount of a polypeptide encoded by a polynucleotide selected from the group consisting of RAG ID NOs:1-281. In one embodiment of the method, the polypeptide is an Fn14 polypeptide. In one embodiment of the method, a composition that upregulates a polypeptide encoded by a polynucleotide selected from the group consisting of RAG ID NOs:1-281 is administered to the subject. In another embodiment of the method, a composition that down-regulates a polypeptide encoded by a polynucleotide selected from the group consisting of RAG ID NOs:1-281 is administered to the subject In another embodiment of the method, the composition comprises a vector that delivers a polynucleotide selected from the group consisting of RAG ID NOs:1-281. In a preferred embodiment of the method, the vector delivers a polynucleotide encoding an Fn14 polypeptide. Also included are polynucleotides encoding variants, analogs, homologs, or fragments of Fn14 polypeptide. In another embodiment of the method, the subject is a human.

In another aspect, the invention includes the use of a compound for the manufacture of a medicament for treatment of a RAG-associated disorder, wherein the compound is selected from the group consisting of a polypeptide encoded by a polynucleotide selected from the RAG ID NOs:1-281. In one embodiment, the compound is an Fn14 polypeptide. In another embodiment of the use, the RAG associated disorder is selected from the group consisting of brain trauma, spinal cord trauma, stroke, cerebral palsy, multiple sclerosis, Parkinson's disease, Alzheimer's disease, ALS, stroke, peripheral neuropathies, brain cancer, neuroblastoma, and reinnervation of transplanted tissue.

In another aspect, the invention includes, a method of treating or preventing a RAG associated disorder, the method comprising administering to a subject in which such treatment or prevention is desired a polypeptide encoded by a polynucleotide selected from the group consisting of RAG ID NOs:1-281 in an amount sufficient to treat or prevent the RAG-associated disorder. In a preferred embodiment of the method, the subject Is administered an Fn14 polypeptide. In another embodiment of the method, the RAG-associated disorder is selected from the group consisting of RAG associated disorder is selected from the group consisting of: brain trauma, spinal cord trauma, stroke, cerebral palsy, multiple sclerosis, Parkinson's disease, Alzheimer's disease, ALS, stroke, peripheral neuropathies, brain cancer, neuroblastoma, and reinnervation of transplanted tissue. In another embodiment of the method, the subject Is a human.

In another aspect, the invention includes a method of treating or preventing a RAG-associated disorder, the method comprising administering to a subject in which such treatment or prevention is desired a polynucleotide selected from the group consisting of RAG ID NOs:1-281 in an amount sufficient to treat or prevent the RAG-associated disorder in the subject. In a preferred embodiment of the invention, the subject is administered a polynucleotide encoding an Fn14 polypeptide (RAG ID NO: 6). In another embodiment of the method the RAG-associated disorder is selected from the group consisting of RAG-associated disorder is selected from the group consisting of: brain trauma, spinal cord trauma, stroke, cerebral palsy, multiple sclerosis, Parkinson's disease, Alzheimer's disease, ALS, stroke, peripheral neuropathies, brain cancer, neuroblastoma, and reinnervation of transplanted tissue. In another embodiment of the method, the subject is a human.

In another aspect, the invention includes a method of treating a pathological state in a mammal, the method comprising administering to the mammal a compound in an amount that Is sufficient to alleviate the pathological state, wherein the compound Is a compound having an amino acid sequence at least 90% identical to a compound comprising a polypepbde encoded by a polynucleotide selected from the group consisting of RAG ID NOs:1-281. In a preferred embodiment of the method, the compound is a compound having an amino acid sequence at least 90% identical to an Fn14 polypeptide (*RAG ID NO:6)

In another aspect, the invention includes a method of treating a RAG-associated disorder in a mammal, the method comprising administering to the mammal at least one compound which modulates the expression or activity of a polypeptide encoded by a polynucleotide selected from the group consisting of RAG ID NOs:1-281. In a preferred embodiment of the method, the at least one compound modulates the expression or activity of an Fn14 polypeptide. In another embodiment of the method, the RAG-associated disorder is selected from the group consisting of: brain trauma, spinal cord trauma, stroke, cerebral palsy, multiple sclerosis, Parkinson's disease, Alzheimer's disease, ALS, stroke, peripheral neuropathies, brain cancer, neuroblastoma, and reinnervation of transplanted tissue.

In another aspect, the invention includes, a method of identifying a compound which binds to a polypeptide encoded by a polynucleotide selected from the group consisting of RAG ID NOs:1-281, the method comprising the steps of, a) providing a candidate compound; b) contacting the candidate compound with the polypeptide under conditions which a complex is formed between the candidate compound and the polypeptide; c) incubating the complex under conditions where co-crystals of the complex form; d) determining the structural atomic coordinates of the complex by x-ray diffraction; and modeling the structure of the complex to determine the binding of the candidate compound to the polypeptide. In a preferred embodiment of the method is a method to identify a compound which binds to an Fn14 polypeptide (RAG ID NO:6). In another aspect, the Invention includes a crystalline preparation of a polypeptide encoded by a polynucleotide selected from the group consisting of RAG ID Nos:1-281 and a test compound prepared by this method. In a preferred embodiment, the crystalline preparation comprising an Fn14 polypeptide.

In another aspect, the invention includes a method of identifying a compound which binds to a polypeptide encoded by a polynucleotide selected from the group consisting of RAG ID NOs:1-281, the method comprising the steps of: a) providing a candidate compound; b) contacting the candidate compound with the polypeptide encoded by a polynucleotide selected from the group consisting of RAG ID NOs: 1-281 under conditions which a complex is formed between the candidate compound and the polypeptide; c) determining the binding or structure of the complex by methods of nuclear magnetic resonance spectroscopy or mass; and optionally e) modeling the structure of the complex. In a preferred embodiment of the method, the compound binds to an Fn14 polypeptide (RAG ID NO:6).

In another aspect, the invention includes a transgenic non-human mammal, having genomically-integrated in non-human mammal cells, comprising a nucleic acid compound having a first segment which is a regulatory region and a second segment which is a polynucleotide sequence encoding a compound selected from the group consisting of RAG ID NOs:1-281, wherein the first segment is operably linked to the second segment. In one embodiment, the second segment comprises a polynucleotide sequence which encodes an Fn14 compound. In another embodiment, the transgenic non-human mammal is a transgenic mouse. In another embodiment,the first segment is a regulatable expression element or elements which are subject to cell- or Issue-specific regulation. In another aspect, the invention includes tissue or cells derived, or cultured from, the non-human transgenic mammal described above.

In another aspect, the invention includes, a transgenic knockout non-human mammal whose genome comprises a homozygous or heterozygous disruption in at least one RAG gene selected from the group consisting of RAG ID NOs:1-281, wherein the homozygous disruption prevents the expression of a functional RAG polypeptide, and wherein the heterozygous disruption reduces the expression of a functional RAG polypeptide in the knockout mouse. In a preferred embodiment, the at least one RAG gene is an Fn14 gene. In another embodiment, the transgenic knockout non-human mammal is a transgenic mouse. In another aspect, the invention includes tissue or cells derived, or cultured from, the non-human knockout transgenic non-human mammal described above.

In another aspect, the invention includes a method for producing a cell in which the expression of a RAG gene selected from the group consisting of RAG ID NOs:1-281 expression is decreased, wherein the method comprises the steps of: introducing an siRNA expression system into a host cells; and selecting the host cells in which the siRNA expression system is introduced. In a preferred embodiment of the method, the expression of an Fn14 gene is decreased.

In another aspect, the invention includes a cell maintaining an siRNA expression system in which the expression of a RAG polypeptide encoded by a RAG gene selected from the group consisting of RAG ID NOs:1-281 expression is decreased. In a preferred embodiment, the cell maintains an siRNA expression system in which the expression of an Fn14 polypeptide (RAG ID NO:6) Is decreased. In one embodiment, the cell is a mammalian cell.

In another aspect, the invention includes an antibody or fragment thereof that binds immunospecifically to a polypeptide encoded by a polynucleotide selected from the group consisting of RAG ID NOs:1-281. In one embodiment, the antibody or fragment thereof binds immunospecifically to an Fn14 polypeptide (RAG ID NO:6). In a preferred embodiment, the antibody is a monoclonal antibody. In another embodiment, the antibody is a humanized antibody.

In another aspect, the invention includes a method of treating a pathological state in a mammal, the method comprising administering to the mammal an anti-RAG antibody described above in an amount sufficient to alleviate the pathological state. In a preferred embodiment of the method, an anti-Fn14 antibody is administered in an amount to alleviate the pathological state.

In another aspect, the invention includes a pharmaceutical composition comprising an an-RAG antibody described above and a pharmaceutically acceptable carrier. In a preferred embodiment, the antibody is an anti-Fn14 antibody.

In another aspect, the invention includes a kit comprising in one or more containers, an anti-RAG antibody and a pharmaceutically acceptable carrier, and instructions for using the contents therein. In one embodiment, in a preferred embodiment, the antibody is an anti-Fn14 antibody.

In another aspect, the invention includes a method of detecting a RAG polypeptide encoded by a polynucleotide selected from the group consisting of RAG ID Nos:1-281, the method comprising: (a) providing a test sample; (b) contacting the test sample with an anti-RAG antibody of claim 94 under conditions which the ant-RAG antibody complexes with the RAG polypeptide to form an anti-RAG antibody/RAG polypeptide complexes; (c) detecting the anti-RAG antibody/RAG polypeptide complexes; and (d) quantifying the anti-RAG antibody/RAG polypeptide complexes in the test sample. In one embodiment of the method, the method is a method of detecting an Fn14 polypeptide (RAG ID NO:6).

In another aspect, the invention includes a method of identifying a compound that binds to a polypeptide encoded by a polynucleotide selected from the group consisting of RAG ID NOs:1-281, the method comprising: (a) contacting the compound with the polypeptide encoded by a polynucleotide selected from the group consisting of RAG ID NOs: 1-281; and (b) determining whether the compound binds to the compound of the polypeptide encoded by a polynucleotide selected from the group consisting of RAG ID Nos:1-281. In a preferred embodiment the method, a compound Is identified that binds to FN14 polypeptide (RAG ID NO;6).

In another aspect, the invention includes a method for determining the presence of, or predisposition to, a disease associated with altered levels of a RAG polypeptide encoded by a polynudeotide selected from the group consisting of RAG ID NOs:1-281 in a first mammalian subject, the method comprising: (a) providing a test sample from the first mammalian subject; (b) contacting the test sample from the first mammalian subject with compound that binds the RAG polypeptide; (c) detecting the level of compound/RAG polypeptide complex; (d) quantifying the level of expression of the RAG polypeptide in the sample from the first mammalian subject; and (e) comparing the amount of the RAG polypeptide in the sample of step (a) to the amount of polypeptide present in a control sample from a second mammalian subject known not to have, or not to be predisposed to, the disease, wherein an alteration in the expression level of the RAG polypeptide in the first subject as compared to the control sample indicates the presence of or predisposition to the disease. In a preferred embodiment the method, the disease Is associated with altered level of FN14 polypeptide (RAG ID NO;6).

### BRIEF DESCRIPTION OF THE DRAWINGS

The present invention will be further understood from the following description with reference to the figures, in which:

FIG. 1 shows Northern blots and photomicrographs illustrating that the myosin-X, SOX11, and FLRT3 genes are induced in DRG neurons by axotomy. Panel A, Northern blot analysis shows mRNA expression of myosin-X, SOX11, and FLRT3 in uninjured E15, uninjured P3, adult axotomized (Axo), and adult contralateral (Con) L46 mouse DRG neurons. Samples were collected 7 d after axotomy. Relative signal intensity for indicated bands is reported at the bottom of each lane. The ethidium bromide staining pattern of the 18S ribosomal RNA Is shown to demonstrate the quantity of RNA loaded, and size markers are shown at the left. Panel B, in situ hybridization shows colocalization of SOX11 and FLRT3 transcripts in the fluoro-gold-labeled axotomized DRG (arrowheads) and little mRNA expression in control DRG sections. Scale bar, 50 μm.

FIG. 2 shows Northern blots and photomicrographs illustrating that Fn14 mRNA expression is induced in DRG neurons by axotomy. A, B, Northern blot analysis of Fn14 mRNA expression in uninjured E15, uninjured P3, adult axotomized (Axo), and adult contralateral (Con) L4-6 mouse DRGs. Samples were collected at 7 d (A) or at various times (B) after sciatic nerve injury. The ethidium bromide staining pattern of the 18S ribosomal RNA is shown to demonstrate the quantity of RNA loaded. The migration of 28S and 18S mRNA is shown at left, and the relative signal intensity is reported at the bottom of each lane. C, in situ hybridization shows colocalization of Fn14 mRNA in the fluoro-gold-labeled axotomized DRG (arrowheads) but not in control contralateral DRG sections. Scale bar: four left panels, 250 μm; two right panels, 50 μm. D, Northern blot analysis of TWEAK mRNA expression in uninjured E15, uninjured P3, adult axotomized, and adult contralateral L4-6 mouse DRGs and in proximal or distal injured nerve stumps. Samples were collected 7 d after sciatic nerve transection. The ethidium bromide staining pattern of the 18S ribosomal RNA is shown to demonstrate the quantity of RNA loaded.

FIG. 3 shows Northern blots illustrating Fn14 and TWEAK mRNA expression in PC12 cells. PC12 cells were treated with NGF for various lengths of time and then harvested. Fn14 (A) and TWEAK (B) mRNA levels were examined by Northern blot. In A, the relative signal intensity for Fn14 mRNA is reported at the bottom of each lane. The ethidium bromide staining pattern of the 18S ribosomal RNA is shown to demonstrate the quantity of RNA loaded.

FIG. 4 shows schematic diagrams, photomicrographs, and immunoblots illustrating that Fn14 overexpression in PC12 cells promotes neurite outgrowth. Panel A, A schematic of Fn14 protein features and the mutant forms of the protein that were expressed here. Panel B, PC12 cells were cultured for 24 hr after infection with HSV-Fn14wild-GFP, HSV-Fn14ECT-GFP, HSV-Fn14END-GFP, and HSV-GFP. Cellular F-actin was visualized with rhodamine-phalloidin staining. Panel C, Neurite length per cell and the percentage of cells with neurites in PC12 cells after infection. Data are means±SEM from four independent experiments. A significant (p<0.01; Student's two-tailed t test) increase in neurite outgrowth is observed in PC12 cells with HSV-Fn14wild-GFP compared with control cells with HSV-GFP. Panel D, The percentage of PC12 cells with neurites after infection with each HSV virus and the addition of no protein, TWEAK (100 ng/ml), or Fn14-Fc (5 μg/ml) to the culture media. Data are means±SEM from four independent experiments. No significant change of the percentage of neurite-bearing cells is observed by the addition of recombinant TWEAK or Fn14Fc. Panel E, Regulation of DRG outgrowth by Fn14. Dissociated adult mouse DRG neurons were cultured from naive animals or animals undergoing sciatic nerve lesion I week before harvest of the axotomized DRG (preconditioned). The neurons were infected with HSV-Fn14wild-GFP, HSV-Fn14END-GFP, or HSV-GFP. Some HSV-GFP cultures were treated with TWEAK (100 ng/ml). Neurite outgrowth in GFP-expressing neurons was quantitated after 12-18 hr and is expressed as a percentage of the HSV-GFP control value for each culture. A significant (p<0.05; Student's two-tailed t test) increase in neurite outgrowth is observed for naive DRG outgrowth from cells with HSV-Fn14wild-GFP compared with HSV-Fn14END-GFP. Panel F, Expression of recombinant Fn14-GFP protein in DRG neurons. Immunoblot for either Fn14 or GFP demonstrates Fn14 overexpression levels in HSV-Fn14-GFP-infected DRG cultures.

FIG. 5 shows photomicrographs illustrating that Fn14 overexpression in PC12 cells enhances filopodial and growth cone formation. Panel A-E, PC12 cells were cultured for 24 hr after infection with HSV-GFP or HSV-Fn14wild-GFP and examined for GFP (green) and F-actin (red, rhodamine-phalloidin). Although HSV-GFP has no effect on cell morphology (A), overexpression of Fn14 induces a number of long filopodia extending from straight cell borders and from angular cell borders (B). Spreading and lamelipodial formation Is also observed in Fn14-expressing PC12 cells (C). Filopodia and lamelipodia are seen at the distal ends of formed growth cones (D, E). Fn14 and F-actin are well colocalized near the cell perimeter and in filopida and lamelipodia (arrows). F, After HSV-Fn14wild-GFP infection, GAP-43 (red) is expressed abundantly in filopodia and lamelipodia and colocalized with Fn14 (arrows). Scale bars, 10 μm.

FIG. 6 shows photomicrographs and a bar graph illustrating that Fn14 promotes growth cone formation in differentiated PC12 cells. PC12 cells were differentiated for 9 d in the presence of NGF and then examined 24 hr after infection with HSV-GFP or HSV-Fn14-GFP. A, Fn14-GFP and GFP fluorescence are shown in green, whereas rhodamine-phalloidin staining of F-actin is red. Fn14 overexpression promotes growth cone formation compared with GFP control. Scale bar, 10 μm. B, The percentage of neurites with growth cones after infection with HSV-GFP or HSV-Fn14-GFP. In some cases, TWEAK (100 ng/ml) or Fn14-Fc (5 μg/ml) was added to the culture media at the time of viral infection as indicated. Overexpression of Fn14 significantly (p<0.01; Student's two-tailed t test) induces growth cone formation compared with GFP control. Neither TWEAK nor Fn14-Fc significantly alters growth cone formation. Data are means±SEM from three independent experiments.

FIG. 7 shows photomicrographs illustrating that Fn14 colocalizes with Cdc42 and Rac1. Cos-7 cells were cotransfected with GFP-tagged Fn14 and myc -tagged wild-type Rho family GTPases. F-actin was visualized with rhodamine-phalloidin, and Rho family GTPases were visualized with 9E10 anti-myc antibody and Cy3-conjugated anti-mouse IgG antibody. A, Fn14 accumulates and colocalizes with F-actin at dorsal ruffles (arrowheads) and leading edge ruffles (arrows) but not at stress fibers (small arrows). B, C, Cdc42 and Rac1 distribution overlap extensively with that of Fn14 at dorsal ruffies (arrows) and leading edge ruffles (arrowheads). D, RhoA colocalizes with Fn14 at leading edge ruffles (arrowheads) but not at dorsal ruffles (arrows). Scale bar, 20 μm.

FIG. 8 shows Western immunoblots illustrating that Fn14 interacts physically with Raci. A, Communoprecipitation of Fn14-HA with myc-tagged wild-type, constitutively active form and dominant-negative form of Rho family GTPases. Myc-tagged Rho family GTPases were precipitated with myc antibody from the lysate of 293T cells cotransfected with Fn14-HA. The precipitate protein and cell lysate were blotted with anti-HA or anti-myc antibody. Fn14 Is coimmunoprecipitated with Raci with no preference to Rac1 activation state. B, Rac1 activity was evaluated by precipitation with agarose beads containing the Cdc42/Rac1 -binding domain of PAK-1. GTP-bound Raci was precipitated from the lysate of 293T cells transfected with Fn14-HA or mock plasmid. The lysate was blotted with anti-Rac1 antibody and anti-HA antibody. The amount of GTP-bound (active) Raci is not altered by overexpression of Fn14.

FIG. 9 is a bar graph illustrating that Fn14-induced PC12 neurite outgrowth requires Rac1 function. Constitutively active (G12V), dominant-negative (T17N), or wild-type Rac1 was coexpressed with Fn14-GFP (Fn14) or GFP in PC12 cells, and neurite outgrowth was assessed. Expression of Rac1G12V+GFP, Rac1wild+Fn14-GFP, or Fn14-GFP alone (+Mock) increased neurite outgrowth significantly, compared with GFP-expressing cells (*p<0.01; Student's two-tailed t test). Coexpression of Rac1G12V with Fn14-GFP increased outgrowth to levels greater than that observed with Rac1 G12V+GFP, Rac1wild+Fn14-GFP, or Fn14-GFP expression (**p<0.05; Student's two-tailed t test). Cells expressing Rac1T17N with Fn14-GFP exhibited outgrowth indistinguishable from control GFP-expressing cells and significantly less outgrowth than those cells expressing Rac1G12V+GFP, Rac1wild+Fn14-GFP, or Fn14-GFP alone (***p<0.05; Student's two-tailed t test). Data are the means+SEM from three independent experiments.

### DETAILED DESCRIPTION OF THE INVENTION

Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

“Axon,” as used herein, refers to a long cellular protrusion from a neuron, whereby efferent (outgoing) action potentials are conducted from the cell body towards target cells.

“Axonal growth,” as used herein, refers to an extension of the long process or axon, originating at the cell body and preceded by the growth cone.

“Neurite,” as used herein, refers to a process growing out of a neuron. As it is sometimes difficult to distinguish a dendrite from an axon in culture, the term neurite is used for both.

“Aromatic amino acid,” as used herein, refers to a hydrophobic amino acid having a side chain containing at least one ring having a conjugated electron system (aromatic group). The aromatic group may be further substituted with substituent groups such as alkyl, alkenyl, alkynyl, hydroxyl, sulfanyl, nitro and amino groups, as well as others. Examples of genetically encoded aromatic amino acids include phenylalanine, tyrosine and tryptophan. Commonly encountered non-genetically encoded aromatic amino acids include phenylglycine, 2-naphthylalanine, beta-2-thienylalanine, 1,2,3,4-tetrahydroisoquinoline-3-carboxylic add, 4-chlorophenylalanine, 2-fluorophenylalanine, 3-fluorophenylalanine and 4-fluorophenylalanine.

“Aliphatic amino acid,” as used herein, refers to an apolar amino acid having a saturated or unsaturated straight chain, branched or cyclic hydrocarbon side chain. Examples of genetically encoded aliphatic amino acids include alanine, leucine, valine and Isoleucine. Examples of non-encoded aliphatic amino acids include norneucine (Nle).

“Acidic amino acid,” as used herein, refers to a hydrophilic amino acid having a side chain pK value of less than 7. Addic amino acids typically have negatively charged side chains at physiological pH due to loss of a hydrogen ion. Examples of genetically encoded acidic amino acids include aspartic acid (aspartate) and glutamic acid (glutamate).

“Basic amino acid,” as used herein, refers to a hydrophilic amino acid having a side chain pK value of greater than 7. Basic amino acids typically have positively charged side chains at physiological pH due to association with hydronium Ion. Examples of genetically encoded basic amino acids include arginine, lysine and histidine. Examples of non-genetically encoded basic amino acids include the non-cyclic amino adds omithine, 2,3-diaminopropionic acid, 2,4-diaminobutyric acid and homoarginine.

“Polar amino acid,” as used herein, refers to a hydrophilic amino acid having a side chain that is uncharged at physiological pH, but which has a bond in which the pair of electrons shared in common by two atoms is held more closely by one of the atoms. Examples of genetically encoded polar amino adds include asparagine and glutarnine. Examples of non-genetcally encoded polar amino acids include citrulline, N-acetyl lysine and methionine sulfoxide.

As will be appreciated by those having skill in the art, the above classification are not absolute—several amino acids exhibit more than one characteristic property, and can therefore be included in more than one category. For example, tyrosine has both an aromatic ring and a polar hydroxyl group. Thus, tyrosine has dual properties and can be included in both the aromatic and polar categories.

A “subject,” as used herein, Is preferably a mammal, such as a human, but can also be an animal, e.g., domestic animals (e.g., dogs, cats and the like), farm animals (e.g., cows, sheep, pigs, horses and the like) and laboratory animals (e.g., rats, mice, guinea pigs and the like).

An “effective amount” of a compound, as used herein, is a quantity sufficient to achieve a desired therapeutic and/or prophylactic effect, for example, an amount which results in the prevention of or a decrease in the symptoms associated with a disease that is being treated, e.g., the diseases associated with the RAGs and RAG polypeptides listed above. The amount of compound administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. Typically, an effective amount of the compounds of the present invention, sufficient for achieving a therapeutic or prophylactic effect, range from about 0.0001 mg per kilogram body weight per day to about 10,000 mg per kilogram body weight per day. Preferably, the dosage ranges are from about 1 mg per kilogram body weight per day to about 100 mg per kilogram body weight per day. The compounds of the present invention can also be administered in combination with each other, or with one or more additional therapeutic compounds.

An “isolated” or “purified” polypeptide or polypeptide or biologically-active portion thereof is substantially free of cellular material or other contaminating polypeptides from the cell or Issue source from which the RAG polypeptide is derived, or substantially free from chemical precursors or other chemicals when chemically synthesized.

“Substantially free of cellular material,” as used herein, includes preparations of RAG in which the polypeptide is separated from cellular components of the cells from which it is isolated or recombinantoy-produced. In one embodiment, the language “substantially free of cellular material” includes preparations of RAG polypeptides having less than about 30% (by dry weight) of non-RAG polypeptides (also referred to herein as a “contaminating polypeptide”), more preferably less than about 20% of non-RAG polypeptides, still more preferably less than about 10% of non-RAG polypeptides, and most preferably less than about 5% of non-RAG polypeptides. When the RAG polypeptide or biologically-acfive portion thereof is recombinantly-produced, it is also preferably substantially free of culture medium, i.e., culture medium represents less than about 20%, more preferably less than about 10%, and most preferably less than about 5% of the volume of the RAG polypeptides preparation.

The language “substantially free of chemical precursors or other chemicals,” as used herein, includes preparations of RAG polypeptide in which the polypeptide is separated from chemical precursors or other chemicals that are involved in the synthesis of the polypeptide. In one embodiment, the language “substantially free of chemical precursors or other chemicals” includes preparations of RAG polypeptides having less than about 30% (by dry weight) of chemical precursors or non-RAG chemicals, more preferably less than about 20% chemical precursors or non-RAG chemicals, still more preferably less than about 10% chemical precursors or non-RAG chemicals, and most preferably less than about 5% chemical precursors or non-RAG chemicals.

The term “variant,” as used herein, refers to a compound that differs from the compound of the present invention, but retains essential properties thereof. A non-limiting example of this is a polynucleotide or polypeptide compound having conservative substitutions with respect to the reference compound, commonly known as degenerate variants. Another non-limiting example of a variant is a compound that is structurally different, but retains the same active domain of the compounds of the present invention. Variants include N-terminal or C-terminal extensions, capped amino acids, modifications of reactive amino acid side chain functional groups, e.g., branching from lysine residues, pegylation, and/or truncations of a polypeptide compound. Generally, variants are overall closely similar, and in many regions, identical to the compounds of the present invention. Accordingly, the variants may contain alterations in the coding regions, non-coding regions, or both.

A “small molecule,” as used herein, refers to a composition that has a molecular weight of less than about 5 kDa and more preferably less than about 2 kDa. Small molecules can be, e.g., nucleic acids, peptides, polypeptides, glycopeptides, peptidomimetics, carbohydrates, lipids, lipopolysaccharides, combinations of these, or other organic or inorganic molecules.

A ∫RAG compound” as used herein, can be, e.g., a polypeptde, peptide, polynucleotide, anti-RAG antibody or a small molecule as well as variants, analogs, homologs, or fragments thereof.

### DETAILED DESCRIPTION

### I. COMPOSITIONS OF THE INVENTION

A. Identification of the RAGs of the Invention by Microarray Expression Profile of Nerve Injury-Related Genes

In previous experiments, incyte microarrays consisting of PCR products derived from Unigene expressed sequence tags (ESTs) were used to compare dorsal root ganglia (DRG) mRNAs before and 7 d after sciatic nerve transection (Bonilla et al., 2002). Hybridization signals were detected for only 1500 of 8000 cDNAs on these arrays, so that a very incomplete survey of RAGs was obtained. In subsequent studies, Affymetrix arrays covering 6000 mouse fullength cDNAs and 30,000 EST clusters were used in the same experimental paradigm (Bonilla et al., J. Neurosci., 23(29): 9675-9686, 2003, incorporated by reference herein in its entirety). Not only did this provide greater coverage of the genome, but the method is more sensitive, yielding detectable hybridization signals for 24% of genes. In an initial comparison at 7 d after injury, it was dear that there are many more differentially ex,pressed genes detected by this method.

Table 1 summarizes the 281 genes newly detected in these studies as RAGs of the present invention.

TABLE 1
Gene expression changes associated changes in L3-5 DRG neurons one week after
ipsilateral sciatic nerve transection

| RAG | | Fold | |
|---|---|---|---|
| ID NO: | Common Name | Change | Descriptions |
| 1 | Complement C1q, c chain | 16.9 | Cluster Incl X66295: Complement component 1, q |
| | | | subcomponent, c polypeptide /cds = (38, 778) |
| | | | /gb = X66295 /gi = 50228 /ug = Mm.3453 /len = 1019 |
| | | | /STRA = for |
| 2 | Complement C1q, b chain | 15.1 | Cluster Incl M22531: Mouse complement C1q B chain |
| | | | mRNA, complete cds /cds = (156, 917) /gb = M22531 |
| | | | /gl = 192266 /ug = Mm.2570 /len = 1059 /STRA = for |
| 3 | MHC class II antigen A, alpha | 13.4 | Cluster Incl X52643: Histocompatibility 2, class II |
| | | | antigen A, alpha /cds = (0, 764) /gb = X52643 /gi = 51526 |
| | | | /ug = Mm.14176 /len = 942 /STRA = for |
| 4 | lysozyme P | 13.3 | Cluster Incl X51547: Mouse Lzp-s mRNA for lysozyme |
| | | | P (EC 3.2.1.17) /cds = (0, 446) /gb = X51547 /gi = 49675 |
| | | | /ug = Mm.57081 /len = 447 /STRA = for |
| 5 | LOMP, LIM only domain | 13.1 | Cluster Incl AW047919: UI-M-BH1-aln-c-05-0-UI.s1 |
| | protein, KIAA 0858 | | Mus musculus cDNA, 3 end /clone = UI-M-BH1-aln-c- |
| | | | 05-0-UI /clone_end = 3 /gb = AW047919 /gi = 5908448 |
| | | | /ug = Mm.6772 /len = 413 /STRA = for |
| 6 | Fn14 | 9.6 | Cluster Incl AI853558: UI-M-BH0-ajd-h-06-0-UI.s1 Mus |
| | | | musculus cDNA, 3 end /clone = UI-M-BHO-ajd-h-06-0- |
| | | | UI /clone_end = 3 /gb = AI853558 /gi = 5497464 |
| | | | /ug = Mm.28518 /len = 385 /STRA = for |
| 7 | FLRT-3 | 9.5 | Cluster Incl AI447780: mq83h12.xl Mus musculus |
| | | | cDNA, 3 end /clone = IMAGE-585383 /clone_end = 3 |
| | | | /gb = AI447780 /gi = 4306384 /ug = Mm.30970 /len = 273 |
| | | | /STRA = for |
| 8 | ADAM-8 | 9.1 | Cluster Incl X13335: A disintegrin and metalloprotease |
| | | | domain (ADAM) 8 /cds = (17, 2497) /gb = X13335 |
| | | | /gi = 1216173 /ug = Mm.15969 /len = 3034 /STRA = for |
| 9 | Unknown | 8.7 | Cluster Incl AW049787: UI-M-BH1-anm-e-03-0-UI.s1 |
| | | | Mus musculus cDNA, 3 end /clone = UI-M-BH1-anm-e- |
| | | | 03-0-UI /clone_end = 3 /gb = AW049787 /gi = 5910316 |
| | | | /ug = Mm.34126 /len = 434 /STRA = for |
| 10 | Unknown | 8.2 | Cluster Incl AW208098: EST100001 Mus musculus |
| | | | cDNA /gb = AW208098 /gi = 6507604 /ug = Mm.23645 |
| | | | /len = 2426 /STRA = rev |
| 11 | Sox11 | 8.0 | Cluster Incl AF009414: Mus musculus SOX11 (Sox11) |
| | | | mRNA, complete cds /cds = (288, 1475) /gb = AF009414 |
| | | | /gi = 2581939 /ug = Mm.6238 /len = 2896 /STRA = for |
| 12 | Aldose-reductase related | 7.8 | Cluster Incl U04204: Fibroblast growth factor regulated |
| | protein | | protein /cds = (67, 1017) /gb = U04204 /gi = 463376 |
| | | | /ug = Mm.5378 /len = 1304 /STRA = for |
| 13 | Unknown | 7.7 | Cluster Incl X78445: M. musculus Cyp1-b-1 mRNA for |
| | | | cytochrome P450 /cds(179, 1810) /gb = X78445 |
| | | | /gi = 506463 /ug = Mm.4443 /len = 2006 /STRA = for |
| 14 | lysozyme M | 7.4 | Cluster Incl M21050: Mouse lysozyme M gene |
| | | | /cds = (30, 476) /gb = M21050 /gi = 198950 /ug = Mm.654 |
| | | | /len = 1264 /STRA = for |
| 15 | Glucosaminyltransferase, | 7.4 | Cluster Incl U68182; Glucosaminyltransferase, I- |
| | I-branching enzyme | | branching enzyme /cds = (236, 1438) /gb = U68182 |
| | | | /gi = 1763028 /ug = Mm.23803 /len = 2393 /STRA = rev |
| 16 | MHC class II H2-I-A-beta | 7.4 | Cluster Incl M21932: Mouse MHC class II H2-I-A-beta |
| | | | gene (k haplotype), 3 end /cds = (0, 697) /gb = M21932 |
| | | | /gi = 199495 /ug = Mm.6716 /len = 740 /STRA = for |
| 17 | Unknown | 7.1 | Cluster Incl AW125664: UI-M-BH2.2-aqi-c-07-0-UI.s1 |
| | | | Mus musculus cDNA, 3 end /clone = UI-M-BH2.2-aqi-c- |
| | | | 07-0-UI /clone_end = 3 /gb = AW125664 /gi = 6101194 |
| | | | /ug = Mm.28054 /len = 314 /STRA = for |
| 18 | MT3-MMP, membrane-type | 7.0 | Cluster Incl AI464339: mv72b10.x1 Mus musculus |
| | matrix metalloprotease | | cDNA, 3 end /clone = IMAGE-660571 /clone_end = 3 |
| | | | /gb = AI464339 /gi = 4318369 /ug = Mm.32759 /len = 237 |
| | | | /STRA = for |
| 19 | Myosin X | 7.0 | Cluster Incl AJ249706: Mus musculus partial mRNA for |
| | | | myosin X (myo 10 gene) /cds = (0, 6092) /gb = AJ249706 |
| | | | /gi = 5921504 /ug = Mm.60590 /len = 6093 /STRA = for |
| 20 | Unknown | 7.0 | Cluster Incl X75926: ATP-binding cassette 1 |
| | | | /cds = (263, 6868) /gb = X75926 /gi = 495256 /ug = Mm.369 |
| | | | /len = 7878 /STRA = rev |
| 21 | Sox11 | 6.8 | Cluster Incl AI836553: UI-M-APO-abe-e-10-0-UI.s1 |
| | | | Mus musculus cDNA, 3 end /clone = UI-M-AP0-abe-e- |
| | | | 10-0-U1 /clone_end = 3 /gb = AI836553 /gi = 5470766 |
| | | | /ug = Mm.40715 /len = 450 /STRA = rev |
| 22 | Sox11 | 6.7 | Cluster Incl AW107922: um24d07.xl Mus musculus |
| | | | cDNA, 3 end /clone = IMAGE-2225485 /clone_end = 3 |
| | | | /gb = AW107922 /gi = 6078722 /ug = Mm.80595 /len = 576 |
| | | | /STRA = rev |
| 23 | Unknown | 6.6 | Cluster Incl AA217038: mu88c12.rl Mus musculus |
| | | | cDNA, 5 end /clone = IMAGE-652630 /clone_end = 5 |
| | | | /gb = AA217038 /gi = 1826049 /ug = Mm.30694 /len = 366 |
| | | | /STRA = for |
| 24 | IGF-binding protein 3 | 6.3 | Cluster Incl AI842277: UI-M-AM1-afz-e-02-0-UI.s1 Mus |
| | | | musculus cDNA, 3 end /clone = UI-M-AM1-afz-e-02-0- |
| | | | UI /clone_end = 3 /gb = AI842277 /gi = 5476530 |
| | | | /ug = Mm.29254 /len = 475 /STRA = for |
| 25 | GADD45, growth arrest and | 6.2 | Cluster Incl U00937: Mus musculus GADD45 protein |
| | DNA damage | | (gadd45) gene, complete cds /cds = (0, 497) |
| | | | /gb = U00937 /gi = 392933 /ug = Mm.1236 /len = 498 |
| | | | /STRA = for |
| 26 | Protein tyrosine phosphatase | 5.9 | Cluster Incl AI551067: vx35c07.x1 Mus musculus |
| | | | cDNA, 3 end /clone = IMAGE-1277196 /clone_end = 3 |
| | | | /gb = AI551067 /gi = 4483430 /ug = Mm.28473 /len = 395 |
| | | | /STRA = for |
| 27 | Sox11 | 5.8 | Cluster Incl AI835269: UI-M-AQ0-aab-c-07-0-UI.s1 |
| | | | Mus musculus cDNA, 3 end /clone = UI-M-AQ0-aab-c- |
| | | | 07-0-UI /clone_end = 3 /gb = AI835269 /gi = 5469482 |
| | | | /ug = Mm.41702 /len = 371 /STRA = for |
| 28 | ion channel homolog RIC | 5.7 | Cluster Incl U72680: Mus musculus ion channel |
| | | | homolog RIC mRNA, complete cds /cds = (48, 584) |
| | | | /gb = U72680 /gi = 1872490 /ug = Mm.1870 /len = 797 |
| | | | /STRA = for |
| 29 | MPS1, mitogen-activated | 5.7 | Cluster Incl L20315: Mus musculus MPS1 gene and |
| | protein kinase | | mRNA, 3 end /cds = (155, 2142) /gb = L20315 |
| | | | /gi = 431419 /ug = Mm.3999 /len = 4231 /STRA = for |
| 30 | Annexin-1 | 5.6 | Cluster Incl M69260: Lipocortin 1 /cds = (14, 1054) |
| | | | /gb = M69260 /gi = 198844 /ug = Mm.14860 /len = 1340 |
| | | | /STRA = for |
| 31 | Unknown | 5.4 | Cluster Incl AW123573: UI-M-BH2.1-aqb-h-11-0-UI.s1 |
| | | | Mus musculus cDNA, 3 end /clone = UI-M-BH2.1-aqb- |
| | | | h-11-0-UI /clone_end = 3 /gb = AW123573 /gi = 6099 103 |
| | | | /ug = Mm.29338 /len = 375 /STRA = for |
| 32 | Unknown | 5.1 | Cluster Incl AW125276: UI-M-Bi-12.1-apy-a-02-0-UI.s1 |
| | | | Mus musculus cDNA, 3 end /clone = UI-M-BH2.1-apy- |
| | | | a-02-0-UI /clone_end = 3 /gb = AW125276 /gi = 6100806 |
| | | | /ug = Mm.32463 /len235 /STRA = for |
| 33 | Complement C1q, a chain | 5.0 | Cluster Incl X58861: Complement component 1, q |
| | | | subcomponent, alpha polypeptide /cds = (29, 766) |
| | | | /gb = X58861 /gi = 50226 /ug = Mm.370 /len = 1033 |
| | | | /STRA = for |
| 34 | mitogen-responsive 96 kDa | 5.0 | Cluster Incl U18869: Mus musculus mitogen- |
| | phosphoprotein p96 mRNA | | responsive 96 kDa phosphoprotein p96 mRNA, |
| | | | alternatively spliced p67 mRNA, and alternatively |
| | | | spliced p93 mRNA, complete cds /cds = (216, 2516) |
| | | | /gb = U18869 /gi = 1176369 /ug = Mm.34248 /len = 3347 |
| | | | /STRA = for |
| 35 | TYRO protein tyrosine kinase | 4.9 | Cluster Incl AF024637: TYRO protein tyrosine kinase |
| | binding protein | | binding protein /cds = (0, 344) /gb = AF024637 |
| | | | /gi = 2906001 /ug = Mm.46301 /len = 345 /STRA = for |
| 36 | BTEB-1 transcription factor | 4.9 | Cluster Incl Y14296: Mus musculus mRNA for BTEB-1 |
| | | | transcription factor /cds = (0, 734) /gbY14296 |
| | | | /gi = 2276328 /ug = Mm.19788 /len = 1639 /STRA = for |
| 37 | GPR2, guanosine | 4.7 | Cluster Incl AW121086: UI-M-BH2.3-aof-h-02-0-UI.s1 |
| | monophosphate reductase | | Mus musculus cDNA, 3 end /clone = UI-M-BH2.3-aof-h- |
| | isolog | | 02-0-UI /clone_end = 3 /gb = AW121086 /gi = 6096419 |
| | | | /ug = Mm.48670 /len = 384 /STRA = rev |
| 38 | chop-10 | 4.7 | Cluster Incl X67083: M. musculus chop-10 mRNA |
| | | | /cds = (74, 580) /gbX67083 /gi = 50406 /ug = Mm.7549 |
| | | | /len = 782 /STRA = for |
| 39 | mrg1, melanocyte specific- | 4.7 | Cluster Incl Y15163: Mus musculus mRNA for mrg1 |
| | related protein | | protein /cds = (219, 1028) /gb = Y15163 /gi = 3059126 |
| | | | /ug = Mm.9524 /len = 1943 /STRA = for |
| 40 | Annexin-1 | 4.7 | Cluster Incl M69260: Lipocortin 1 /cds = (14, 1054) |
| | | | /gb = M69260 /gi = 198844 /ug = Mm.14860 /len = 1340 |
| | | | /STRA = for |
| 41 | Unknown | 4.6 | Cluster Incl AW049012: UI-M-BH1-amp-e-05-0-UI.s1 |
| | | | Mus musculus cDNA, 3 end /clone = UI-M-BH1-amp-e- |
| | | | 05-0-UI /clone_end = 3 /gb = AW049012 /gi = 5909541 |
| | | | /ug = Mm.44971 /len = 399 /STRA = for |
| 42 | CSF-1 receptor | 4.6 | Cluster Incl X06368: Colony stimulating factor 1 |
| | | | receptor /cds = (75, 3005) /gb = X06368 /gi = 50980 |
| | | | /ug = Mm.22574 /len = 3665 /STRA = for |
| 43 | macrophage colony- | 4.5 | Cluster Incl M21952: Mouse macrophage colony- |
| | stimulating factor | | stimulating factor (4 kb) mRNA, complete cds |
| | | | /cds = (159, 1817) /gb = M21952 /gi = 192804 |
| | | | /ug = Mm.795 /len = 3922 /STRA = for |
| 44 | p21/WAF | 4.5 | Cluster Incl U09507: Cyclin-dependent kinase inhibitor |
| | | | 1A (P21) /cds = (200, 679) /gb = U09507 /gi = 595302 |
| | | | /ug = Mm.34446 /len = 855 /STRA = for |
| 45 | Rnase A4 | 4.5 | Cluster Incl AI840339: UI-M-AJ0-abd-g-01-0-UI.s2 Mus |
| | | | musculus cDNA, 3 end /clone = UI-M-AJ0-abd-g-01-0- |
| | | | UI /clone_end = 3 /gb = AI840339 /gi = 5474552 |
| | | | /ug = Mm.2587 /len = 497 /STRA = for |
| 46 | Growth hormone releasing | 4.4 | Cluster Incl M31658: Growth hormone releasing |
| | hormone | | hormone /cds(91, 402) /gb = M31658 /gi = 193635 |
| | | | /ug = Mm.6317 /len = 503 /STRA = rev |
| 47 | Syndecan 1 | 4.2 | Cluster Incl Z22532: Syndecan 1 /cds = (240, 1175) |
| | | | /gb = Z22532 /gi = 1946227 /ug = Mm.2580 /len = 3054 |
| | | | /STRA = for |
| 48 | Unknown | 4.2 | Cluster Incl AI840035: UI-M-AH0-acu-g-12-0-UI.s1 |
| | | | Mus musculus cDNA, 3 end /clone = UI-M-AH0-acu-g- |
| | | | 12-0-UI /clone_end = 3 /gb = AI840035 /gi = 5474248 |
| | | | /ug = Mm.28024 /len = 435 /STRA = for |
| 49 | AKAP121, Akinase anchoring | 4.2 | Cluster Incl U95146: Mus musculus AKAP12I mRNA, |
| | protein | | complete cds /cds = (197, 2770) /gb = U95146 |
| | | | /gi = 2072306 /ug = Mm.2969 /len = 2858 /STRA = for |
| 50 | Unknown | 4.1 | Cluster Incl AW049806: UI-M-BH1-anm-g-08-0-UI.s1 |
| | | | Mus musculus cDNA, 3 end /clone = UI-M-BH1-anm-g- |
| | | | 08-0-UI /clone_end = 3 /gb = AW049806 /gi = 5910335 |
| | | | /ug = Mm.29274 /len = 457 /STRA = for |
| 51 | Unknown | 4.1 | Cluster Incl AA939922: vq64c03.s1 Mus musculus |
| | | | cDNA, 3 end /clone = IMAGE-1107076 /clone_end = 3 |
| | | | /gb = AA939922 /gi = 3099466 /ug = Mm.31358 /len = 409 |
| | | | /STRA = rev |
| 52 | Glucosamine phosphate | 4.1 | Cluster Incl AW123026: UI-M-BH2.1-apb-f-07-0-UI.s1 |
| | N-acetyltransferase | | Mus musculus cDNA, 3 end /clone = UI-M-BH2.1-apb-f- |
| | | | 07-0-UI /clone_end = 3 /gb = AW123026 /gi = 6098556 |
| | | | /ug = Mm.25336 /len = 442 /STRA = for |
| 53 | Xanthine dehydrogenase | 4.1 | Cluster Incl X75129: Xanthine dehydrogenase |
| | | | /cds = (25, 4032) /gb = X75129 /gi = 473040 /ug = Mm.3043 |
| | | | /len = 4554 /STRA = for |
| 54 | Kinesin heavy chain member | 4.1 | Cluster Incl AF067180: Kinesin heavy chain member |
| | 5C | | 5C /cds = (0, 2870) /gb = AF067180 /gi = 3929109 |
| | | | /ug = Mm.41576 /len = 2871 /STRA = rev |
| 55 | Ribonuclease, angiogenesis | 4.0 | Cluster Incl AI848825: UI-M-AJ1-ahb-b-03-0-UI.s1 Mus |
| | inhibitor | | musculus cDNA, 3 end /clone = UI-M-AJI-ahb-b-03-0- |
| | | | UI /clone_end = 3 /gb = AI848825 /gi = 5492731 |
| | | | /ug = Mm.6586 /len = 342 /STRA = for |
| 56 | Unknown | 4.0 | Cluster Incl AA499308: vi92g08.r1 Mus musculus |
| | | | cDNA, 5 end /clone = IMAGE-919742 /clone_end = 5 |
| | | | /gb = AA499308 /gi = 2234275 /ug = Mm.80391 /len = 434 |
| | | | /STRA = for |
| 57 | L1-like cell adhesion molecule | 3.9 | Cluster Incl AI450598: mq87e09.x1 Mus musculus |
| | | | cDNA, 3 end /clone = IMAGE-585736 /clone_end = 3 |
| | | | /gb = AI450598 /gi = 4298122 /ug = Mm.32332 /len = 270 |
| | | | /STRA = for |
| 58 | Kinesin heavy chain member | 3.9 | Cluster Incl AI13I895: ue97e08.x1 Mus musculus |
| | 5C | | cDNA, 3 end /clone = IMAGE-1499078 /clone_end = 3 |
| | | | /gb = AI131895 /gi = 3601911 /ug = Mm.29381 /len = 397 |
| | | | /STRA = for |
| 59 | L1-like protein | 3.9 | Cluster Incl X94310: M. musculus mRNA for L1-like |
| | | | protein /cds = (295, 3924) /gb = X94310 /gi = 1532034 |
| | | | /ug = Mm.1736 /len = 4437 /STRA = for |
| 60 | GDNF Receptor, alpha | 3.9 | Cluster Incl AF014117: Mus musculus GDNF receptor |
| | | | alpha (GDNFR-alpha) mRNA, complete cds |
| | | | /cds = (913, 2319) /gb = AF014117 /gi = 2624960 |
| | | | /ug = Mm.88367 /len = 2549 /STRA = for |
| 61 | TNFR Intracellular domain | 3.9 | Cluster Incl AI852196: UI-M-BH0-ajc-b-11-0-UI.s1 Mus |
| | interacting protein | | musculus cDNA, 3 end /clone = UI-M-BH0-ajc-b-11-0- |
| | | | UI /clone_end = 3 /gb = AI852196 /gi = 5496102 |
| | | | /ug = Mm.29475 /len = 370 /STRA = for |
| 62 | Iba-1 | 3.9 | Cluster Incl A1854880: UI-M-BH0-akb-f-06-0-UI.s1 |
| | | | Mus musculus cDNA, 3 end /clone = UI-M-BH0-akb-f- |
| | | | 06-0-UI /clone_end = 3 /gb = A1854880 /gi = 5498786 |
| | | | /ug = Mm.41769 /len = 362 /STRA = for |
| 63 | ATPase class protein | 3.8 | Cluster Incl AA710215: vt41f09.r1 Mus musculus |
| | | | cDNA, 5 end /clone = IMAGE-1165673 /clone_end = 5 |
| | | | /gb = AA710215 /gi = 2720133 /ug = Mm.26583 /len = 426 |
| | | | /STRA = rev |
| 64 | RB3 | 3.8 | Cluster Incl AF105222: Mus musculus stathmin-like- |
| | | | protein RB3 mRNA, complete cds /cds = (88, 657) |
| | | | /gb = AF105222 /gi = 4028597 /ug = Mm.35474 /len = 1197 |
| | | | /STRA = rev |
| 65 | ATP binding cassette | 3.7 | Cluster Incl AI845514: UI-M-AO1-aek-d-04-0-UI.s1 |
| | subfamily | | Mus musculus cDNA, 3 end /clone = UI-M-AO1-aek-d- |
| | | | 04-0-UI /clone_end = 3 /gb = AI845514 /gi = 5489420 |
| | | | /ug = Mm.25221 /len = 409 /STRA = for |
| 66 | p21/WAF | 3.7 | Cluster Incl AW048937: UI-M-BH1-amo-d-08-0-UI.s1 |
| | | | Mus musculus cDNA, 3 end /clone = UI-M-BH1-amo-d- |
| | | | 08-0-UI /clone_end = 3 /gb = AW048937 /gi = 5909466 |
| | | | /ug = Mm.2387 /len = 355 /STRA = for |
| 67 | Chromosome 1, open reading | 3.7 | Cluster Incl AA856447: vx01h07.r1 Mus musculus |
| | frame 21 | | cDNA, 5 end /clone = IMAGE-1263229 /clone_end = 5 |
| | | | /gb = AA856447 /gi = 2944749 /ug = Mm.34130 /len = 366 |
| | | | /STRA = for |
| 68 | BALM1, basic leucine zipper | 3.7 | Cluster Incl AA710439: vt42e01.r1 Mus musculus |
| | transcription factor 1 | | cDNA, 5 end /clone = IMAGE-1165752 /clone_end = 5 |
| | | | /gb = AA710439 /gi = 2720357 /ug = Mm.26147 /len = 510 |
| | | | /STRA = for |
| 69 | Protein tyrosine phosphatase, | 3.7 | Cluster Incl U37465: Protein tyrosine phosphatase, |
| | receptor type, O | | receptor type, O /cds = (380, 1597) /gb = U37465 |
| | | | /gi = 1063639 /ug = Mm.4715 /len = 2760 /STRA = rev |
| 70 | Interleukin 1 receptor, type I | 3.7 | Cluster Incl M20656: Interleukin 1 receptor, type I |
| | | | /cds = (224, 1954) /gb = M20658 /gi = 198300 |
| | | | /ug = Mm.896 /len = 4740 /STRA = for |
| 71 | cAMP-dependent protein | 3.7 | Cluster Incl J02935: Mouse cAMP-dependent protein |
| | kinase type II regulatory | | kinase type II regulatory subunit mRNA, 3 end |
| | subunit | | /cds = (0, 1205) /gb = J02935 /gi = 200356 /ug = Mm.3176 |
| | | | /len = 1533 /STRA = for |
| 72 | Unknown | 3.7 | Cluster Incl AA690091: vr79a09.s1 Mus musculus |
| | | | cDNA, 5 end /clone = IMAGE-1134904 /clone_end = 5 |
| | | | /gb = M690091 /gi = 2691027 /ug = Mm.25776 /len = 347 |
| | | | /STRA = for |
| 73 | Caspase 3 | 3.7 | Cluster Incl U54803: Caspase 3, apoptosis related |
| | | | cysteine protease /cds = (15, 848) /gb = U54803 |
| | | | /gi = 1518967 /ug = Mm.34405 /len = 1418 /STRA = rev |
| 74 | Unknown | 3.6 | Cluster Incl AU043023: AU043023 Mus musculus |
| | | | cDNA, 3 end /clone = J1037E06 /clone_end = 3 |
| | | | /gb = AU043023 /gi = 3957258 /ug = Mm.17708 /len = 403 |
| | | | /STRA = rev |
| 75 | Fibroblast growth factor 3 | 3.6 | Cluster Incl Y00848: Fibroblast growth factor 3 |
| | | | /cds = (864, 1601) /gb = Y00848 /gi = 52716 /ug = Mm.4947 |
| | | | /len = 3056 /STRA = for |
| 76 | Unknown | 3.6 | Cluster Incl AW120849: UI-M-BH2.3-aoa-e-10-0-UI.s1 |
| | | | Mus musculus cDNA, 3 end /clone = UI-M-BH2.3-aoa- |
| | | | e-10-0-UI /clone_end = 3 /gb = AW120849 /gi = 6096182 |
| | | | /ug = Mm.87664 /len = 366 /STRA = for |
| 77 | RAGD | 3.6 | Cluster Incl AA600647: vm77b06.r1 Mus musculus |
| | | | cDNA, 5 end /clone = IMAGE-1004243 /clone_end = 5 |
| | | | /gb = AA600647 /gi = 2434272 /ug = Mm.7326 /len = 417 |
| | | | /STRA$$for |
| 78 | cathepsin S | 3.5 | Cluster Incl AJ223208: Mus musculus mRNA for |
| | | | cathepsin S, partial /cds(0, 139) /gb = AJ223208 |
| | | | /gi = 4138249 /ug = Mm.3619 /len = 366 /STRA = for |
| 79 | SEL63 | 3.5 | Cluster Incl C76102: C76102 Mus musculus cDNA, 3 |
| | | | end /clone = J0003E12 /clone_end3 /gb = C76102 |
| | | | /gi = 2516432 /ug = Mm.5454 /len = 598 /STRA = rev |
| 80 | Cathepsin H | 3.5 | Cluster Incl U06119: Cathepsin H /cds = (47, 1048) |
| | | | /gb = U06119 /gi = 454100 /μg = Mm.2277 /len = 1381 |
| | | | /STRA = for |
| 81 | RAG associated protein | 3.4 | Cluster Incl AA881092: vz06c12.r1 Mus musculus |
| | | | cDNA, 5 end /clone = IMAGE-1314934 /clone_end = 5 |
| | | | /gb = AA881092 /gi = 2990402 /ug = Mm.28505 /len = 521 |
| | | | /STRArev |
| 82 | CHMP1.5 | 3.4 | Cluster Incl AW124616: UI-M-BH2.1-apw-a-09-0-UI.s1 |
| | | | Mus musculus cDNA, 3 end /clone = UI-M-BH2.1-apw- |
| | | | a-09-0-UI /clone_end3 /gb = AW124616 /gi = 6100146 |
| | | | /ug = Mm.86545 /len = 438 /STRA = rev |
| 83 | Unknown | 3.4 | Cluster Incl AI874830: u125f09.x1 Mus musculus |
| | | | cDNA, 3 end /clone = IMAGE-2099369 /clone_end = 3 |
| | | | /gb = AI874830 /gi = 5548879 /ug = Mm.22871 /len = 433 |
| | | | /STRA = rev |
| 84 | Unknown | 3.4 | Cluster Incl AI838951: UI-M-AO0-ace-e-06-0-UI.s1 |
| | | | Mus musculus cDNA, 3 end /clone = UI-M-AO0-ace-e- |
| | | | 06-0-UI /clone_end = 3 /gb = AI838951 /gi = 5473164 |
| | | | /ug = Mm.22456 /len = 451 /STRA = for |
| 85 | Unknown | 3.3 | Cluster Incl AI854863: UI-M-BH0-akb-d-12-0-UI.s1 |
| | | | Mus musculus cDNA, 3 end /clone = UI-M-BH0-akb-d- |
| | | | 12-0-UI /clone_end = 3 /gb = AI854863 /gi = 5498769 |
| | | | /ug = Mm.38801 /len = 419 /STRA = for |
| 86 | Unknown, KIAA 0573 | 3.3 | Cluster Incl AW125408: UI-M-BH2.3-aqh-d-12-0-UI.s1 |
| | | | Mus musculus cDNA, 3 end /clone = UI-M-BH2.3-aqh- |
| | | | d-12-0-UI /clone_end = 3 /gb = AW125408 /gi = 6100938 |
| | | | /ug = Mm.29036 /len = 480 /STRA = for |
| 87 | GDNF Receptor, alpha | 3.2 | Cluster Incl AW060572: UI-M-BH1-anq-b-01-0-UI.s1 |
| | | | Mus musculus cDNA, 3 end /clone = UI-M-BH1-anq-b- |
| | | | 01-0-UI /clone_end = 3 /gb = AW060572 /gi = 6008323 |
| | | | /ug = Mm.18758 /len = 443 /STRA = for |
| 88 | Unknown | 3.2 | Cluster Incl AW125203: UI-M-BH2.1-aps-a-10-0-UI.s1 |
| | | | Mus musculus cDNA, 3 end /clone = UI-M-BH2.1-aps- |
| | | | a-10-0-UI /clone_end = 3 /gb = AW125203 /gi = 6100733 |
| | | | /ug = Mm.43208 /len = 365 /STRA = rev |
| 89 | hematopoietic-specific early- | 3.2 | Cluster Incl U23781: Mus musculus hematopoietic- |
| | response A1-d protein | | specific early-response A1-d protein (A1d) gene |
| | | | /cds = (131, 649) /gb = U23781 /gi = 2822192 |
| | | | /ug = Mm.87857 /len = 796 /STRA = for |
| 90 | Unknown | 3.1 | Cluster Incl AA986395: uc82f06.x1 Mus musculus |
| | | | cDNA, 3 end /clone = IMAGE-1432163 /clone_end = 3 |
| | | | /gb = AA986395 /gi = 3168049 /ug = Mm.31409 /len = 757 |
| | | | /STRA = rev |
| 91 | Unknown | 3.1 | Cluster Incl AI839453: UI-M-ANO-aco-h-03-0-UI.s1 |
| | | | Mus musculus cDNA, 3 end /clone = UI-M-ANO-aco-h- |
| | | | 03-0-UI /clone_end = 3 /gb = AI839453 /gi = 5473666 |
| | | | /ug = Mm.40314 /len = 232 /STRA = rev |
| 92 | Unknown | 3.0 | Cluster Incl AW060401: UI-M-BH1-anj-a-06-0-UI.s1 |
| | | | Mus musculus cDNA, 3 end /clone = UI-M-BH1-anj-a- |
| | | | 06-0-UI /clone_end = 3 /gb = AW060401 /gi = 6008152 |
| | | | /ug = Mm.24574 /len = 467 /STRA = for |
| 93 | endothelial monocyte- | 3.0 | Cluster Incl U41341: Mus muscuius endothelial |
| | activating polypeptide I | | monocyte-activating polypeptide I mRNA, complete |
| | | | cds /cds = (0, 296) /gb = U41341 /gi = 1150723 |
| | | | /ug = Mm.383 /len = 331 /STRA = for |
| 94 | Unknown | 3.0 | Cluster Incl AA624105: vq75f04.s1 Mus musculus |
| | | | cDNA, 5 end /clone = IMAGE-1108159 /clone_end = 5 |
| | | | /gb = AA624105 /gi = 2527981 /ug = Mm.37490 /len = 104 |
| | | | /STRA = for |
| 95 | Unknown | 3.0 | Cluster Incl AW228955: up24d03.x1 Mus musculus |
| | | | cDNA, 3 end /clone = IMAGE-2655269 /clone_end = 3 |
| | | | /gb = AW228955 /gi = 6558251 /ug = Mm.89173 /len = 438 |
| | | | /STRA = rev |
| 96 | Unknown. | 3.0 | Cluster Incl AI843031: UI-M-AK1-aeq-e-11-0-UI.s1 |
| | | | Mus musculus cDNA, 3 end /clone = UI-M-AK1-aeq-e- |
| | | | 11-0-UI /clone_end = 3 /gb = AI843031 /gi = 5477244 |
| | | | /ug = Mm.29307 /len361 /STRA = for |
| 97 | Unknown | 3.0 | Cluster Incl AA689001: vs05d07.r1 Mus musculus |
| | | | cDNA, 5 end /clone = IMAGE-1137325 /clone_end = 5 |
| | | | /gb = AA689001 /gi = 2678430 /ug = Mm.86488 /len = 111 |
| | | | /STRA = for |
| 98 | Unknown | 3.0 | Cluster Incl AI449239: mt01d03.x1 Mus musculus |
| | | | cDNA, 3 end /clone = IMAGE-619781 /clone_end = 3 |
| | | | /gb = AI449239 /gi = 4288610 /ug = Mm.32134 /len = 290 |
| | | | /STRA = rev |
| 99 | Unknown | 3.0 | Cluster Incl AW124355: UI-M-BH2.1-apq-h-02-0-UI.s1 |
| | | | Mus musculus c DNA, 3 end /clone = UI-M-BH2.1-apq- |
| | | | h-02-0-UI /clone_end = 3 /gb = AW124355 /gi = 6099885 |
| | | | /ug = Mm.41462 /len = 237 /STRA = for |
| 100 | Tera | 3.0 | Cluster Incl U64033: Mus musculus Tera (Tera) |
| | | | mRNA, complete cds /cds = (81, 914) /gb = U64033 |
| | | | /gi = 1575504 /ug = Mm.18637 /len = 2720 /STRA = for |
| 101 | Unknown | 2.9 | Cluster Incl AI853733: UI-M-BH0-ajg-a-05-0-UI.s1 Mus |
| | | | musculus cDNA, 3 end/clone = UI-M-BH0-ajg-a-05-0- |
| | | | UI /clone_end = 3 /gb = A1853733 /gi = 5497639 |
| | | | /ug = Mm.44011 /len = 410 /STRA = rev |
| 102 | Unknown | 2.9 | Cluster Incl AA657164: vr26f01.r1 Mus musculus |
| | | | cDNA, 5 end /clone = IMAGE-1121785 /clone_end = 5 |
| | | | /gb = AA657164 /gi = 2593318 /ug = Mm.21803 /len = 376 |
| | | | /STRA = for |
| 103 | Unknown | 2.9 | Cluster Incl M98530: Neuron specific gene family |
| | | | member 1 /cds = (0, 557) /gb = M98530 /gi = 200461 |
| | | | /ug = Mm.7414 /len = 558 /STRA = for |
| 104 | Unknown | 2.9 | Cluster Incl AI197601: ud99e04.r1 Mus musculus |
| | | | cDNA, 5 end/clone = IMAGE-1479006 /clone_end = 5 |
| | | | /gb = AI197601 /gi = 3750207 /ug = Mm.46238 $$/len = 396 |
| | | | /STRA = for |
| 105 | Unknown | 2.8 | Cluster Incl AW049506: UI-M-BH1-anf-b-01-0-UI.s1 |
| | | | Mus musculus cDNA, 3 end /clone = UI-M-BH1-anf-b- |
| | | | 01-0-UI /clone_end = 3 /gb = AW049506 /gi = 5910035 |
| | | | /ug = Mm.45173 /len = 402 /STRA = for |
| 106 | Mac-2 antigen | 2.8 | Cluster Incl X16834: Mouse mRNA for Mac-2 antigen |
| | | | /cds = (43, 837) /gb = X16834 /gi = 52986 /ug = Mm.2970 |
| | | | /len = 955 /STRA = for |
| 107 | Unknown | 2.8 | Cluster Incl AI604793: me21h03.x1 Mus musculus |
| | | | cDNA, 3 end /clone = IMAGE-388181 /clone_end = 3 |
| | | | /gb = AI604793 /gi = 4613960 /ug = Mm.38352 /len = 430 |
| | | | /STRA = for |
| 108 | Unknown | 2.8 | Cluster Incl AI852917: UI-M-BH0-aiz-b-04-0-UI.s1 Mus |
| | | | musculus cDNA, 3 end /clone = UI-M-BH0-aiz-b-04-0- |
| | | | UI /clone_end = 3 /gb = AI852917 /gi = 5496823 |
| | | | /ug = Mm.39920 /len = 391 /STRA = rev |
| 109 | Unknown | 2.8 | Cluster Incl AF055638: Mus musculus unknown mRNA |
| | | | /cds = (93, 572) /gb = AF055638 /gi = 4321665 |
| | | | /ug = Mm.9653 /len = 1084 /STRA = for |
| 110 | Unknown | 2.8 | Cluster Incl AW124952: UI-M-BH2.1-apv-a-06-0-UI.s1 |
| | | | Mus musculus cDNA, 3 end/clone = UI-M-BH2.1-apv- |
| | | | a-06-0-UI /clone_end = 3 /gb = AW124952 /gi = 6100482 |
| | | | /ug = Mm.19016 /len = 393 /STRA = for |
| 111 | Unknown | 2.8 | Cluster Incl AA268496: va05f01.r1 Mus musculus |
| | | | cDNA, 5 end /clone = iMAGE-722041 /clone_end = 5 |
| | | | /gb = AA268496 /gi = 1905240 /ug = Mm.86377 /len = 321 |
| | | | /STRA = for |
| 112 | Unknown | 2.8 | Cluster Incl A1842272: UI-M-AM1-afz-d-09-0-U1.s1 |
| | | | Mus musculus cDNA, 3 end /clone = UI-M-AM1-afz-d- |
| | | | 09-0-UI /clone_end = 3 /gb = AI842272 /gi = 5476525 |
| | | | /ug = Mm.21571 /len215 /STRM = for |
| 113 | Unknown | 2.8 | Cluster Inc AA416235: vf38b07.r1 Mus musculus |
| | | | cDNA, 5 end /clone = IMAGE-846037 /clone_end = 5 |
| | | | /gb = AA416235 /gi = 2076503 /ug = Mm.46451 /len = 526 |
| | | | /STRA = for |
| 114 | Unknown | 2.8 | Cluster Incl AA989939: ua51e03.r1 Mus musculus |
| | | | cDNA, 5 end /clone = IMAGE-1350268 /clone_end = 5 |
| | | | /gb = AA989939 /gi = 3175303 /ug = Mm.97106 /len = 294 |
| | | | /STRA = for |
| 115 | Eukaryotic translation | 2.8 | Cluster Incl U28656: Eukaryotic translation initiation |
| | initiation factor 4E binding | | factor 4E binding protein 1 /cds = (50, 403) /gb = U28656 |
| | protein 1 | | /gi = 881557 /ug = Mm.6700 /len = 793 /STRA = for |
| 116 | thymic shared antigen-1 | 2.7 | Cluster Incl U47737: Mus musculus thymic shared |
| | (TSA-1) | | antigen-1 (TSA-1) gene, complete cds /cds = (106, 498) |
| | | | /gb = U47737 /gi = 1199649 /ug = Mm.788 /len = 1117 |
| | | | /STRA = for |
| 117 | Calcium channel, voltage- | 2.7 | Cluster Incl U73487: Calcium channel, voltage- |
| | dependent, L type, alpha 2 | | dependent, L type, alpha 2 delta subunit |
| | delta subunit | | /cds = (230, 3484) /gb = U73487 /gi = 1905824 |
| | | | /ug = Mm.30841 /len = 3882 /STRA = for |
| 118 | Unknown | 2.7 | Cluster Incl AI591446: vt11e04.x1 Mus musculus |
| | | | cDNA, 3 end /clone = IMAGE-1162782 /clone_end = 3 |
| | | | /gb = AI591446 /gi = 4600494 /gi = Mm.74665 /len = 462 |
| | | | /STRA = rev |
| 119 | Unknown | 2.7 | Cluster Incl AW212870: uo68f05.x1 Mus musculus |
| | | | cDNA, 3 end /clone = IMAGE-2647713 /clone_end = 3 |
| | | | /gb = AW212870 /gi = 6519015 /ug = Mm.79307 /len = 482 |
| | | | /STRA = for |
| 120 | M. musculus (Balb/C) Tx01 | 2.6 | Cluster Incl Z31362: M. musculus (Balb/C) Tx01 mRNA |
| | | | /cds = (0, 159) /gb = Z31362 /gi = 467582 /ug = Mm.1364 |
| | | | /len = 228 /STRA = for |
| 121 | Unknown | 2.6 | Cluster Incl A1846501: UI-M-AN1-aff-c-09-0-UI.s1 Mus |
| | | | musculus cDNA, 3 end /clone = UI-M-AN1-aff-c-09-0-UI |
| | | | /clone_end = 3 /gb = A1846501 /gi = 5490407 |
| | | | /ug = Mm.33892 /len = 341 /STRA = for |
| 122 | Unknown | 2.6 | Cluster Incl A1324224: mi26g10.x1 Mus musculus |
| | | | cDNA, 3 end /clone = IMAGE-464706 /clone_end = 3 |
| | | | /gb = AI324224 /gi = 4058653 /ug = Mm.98647 /len = 380 |
| | | | /STRA = rev |
| 123 | Unknown | 2.6 | Cluster Incl AW120831: UI-M-BH2.3-aoa-d-03-0-UI.s1 |
| | | | Mus musculus cDNA, 3 end /clone = UI-M-BH2.3-aoa- |
| | | | d-03-0-UI /clone_end = 3 /gb = AW120831 /gi = 6096164 |
| | | | /ug = Mm.34139 /len = 356 /STRA = for |
| 124 | Unknown | 2.6 | Cluster Incl AI122366: uc6lc03.r1 Mus musculus |
| | | | cDNA, 5 end /clone = IMAGE-1430116 /clone_end = 5 |
| | | | /gb = AI122366 /gi = 3522690 /ug = Mm.86495 /len = 526 |
| | | | /STRA = for |
| 125 | Unknown | 2.6 | Cluster Incl AW108467: um21e10.x1 Mus musculus |
| | | | cDNA, 3 end /clone = IMAGE-2225226 /clone_end = 3 |
| | | | /gb = AW108467 /gi = 6O79267 /ug = Mm.26230 /len = 514 |
| | | | /STRA = rev |
| 126 | Unknown | 2.6 | Cluster Incl Z11974: Mannose receptor, C type 1 |
| | | | /cds(99, 4469) /gb = Z11974 /gi = 52997 /ug = Mm.2019 |
| | | | /len = 5085 /STRA = for |
| 127 | Unknown | 2.6 | Cluster Incl AA692431: vt59b12.r1 Mus musculus |
| | | | cDNA, 5 end /clone = IMAGE-1167359 /clone_end = 5 |
| | | | /gb = AA692431 /gi = 2693369 /ug = Mm.25230 /len = 319 |
| | | | /STRA = for |
| 128 | Unknown | 2.6 | Cluster Incl D85926: Mus musculus mRNA for Sh3yl1, |
| | | | complete cds /cds = (41, 1063) /gb = D85926 |
| | | | /gi = 1944388 /ug = Mm.2090 /len = 1226 /STRA = for |
| 129 | Unknown | 2.6 | Cluster Incl AW261813: um92h08.x1 Mus musculus |
| | | | cDNA, 3 end /clone = IMAGE-2332287 /clone_end = 3 |
| | | | /gb = AW261813 /gi = 6638626 /ug = Mm.89407 /len = 590 |
| | | | /STRA = rev |
| 130 | Unknown | 2.6 | Cluster Incl AA388212: vc24b05.r1 Mus musculus |
| | | | cDNA, 5 end /clone = IMAGE-775473 /clone_end = 5 |
| | | | /gb = AA388212 /gi = 2O41239 /ug = Mm.32488 /len = 473 |
| | | | /STRA = for |
| 131 | Unknown | 2.6 | Cluster Incl X95503: M. musculus mRNA for zinc finger |
| | | | protein (cell line AtT20) /cds = (534, 2537) /gb = X95503 |
| | | | /gi = 2326719 /ug = Mm.42200 /len = 2771 /STRA = for |
| 132 | Unknown | 2.5 | Cluster Incl AI592403: vs77f11.y1 Mus musculus |
| | | | cDNA, 5 end /clone = IMAGE-1152333 /clone_end = 5 |
| | | | /gb = AI592403 /gi = 4601451 /ug = Mm.79898 /len503 |
| | | | /STRA = for |
| 133 | Unknown | 2.5 | Cluster Incl AA288297: mr49d04.r1 Mus musculus |
| | | | cDNA, 5 end /clone = IMAGE-600775 /clone_end = 5 |
| | | | /gb = AA288297 /gi = 1938032 /ug = Mm.86393 /len = 300 |
| | | | /STRA = for |
| 134 | Unknown | 2.5 | Cluster Incl AI850713: UI-M-BG1-ail-d-10-0-UI.s1 Mus |
| | | | musculus cDNA, 3 end /clone = UI-M-BG1-ail-d-10-0-UI |
| | | | /clone_end = 3 /gb = AI850713 /gi = 5494619 |
| | | | /ug = Mm.24984 /len = 321 /STRA = rev |
| 135 | Unknown | 2.5 | Cluster Incl AI413566: mb80g02.x1 Mus musculus |
| | | | cDNA, 3 end /clone = IMAGE-335762 /clone_end = 3 |
| | | | /gb = AI413566 /gi = 4257070 /ug = Mm.22976 /len = 355 |
| | | | /STRA = rev |
| 136 | Unknown | 2.5 | ClusterIncl AW047083: UI-M-BH1-alr-f-05-0-UI.s1 Mus |
| | | | musculus cDNA, 3 end /clone = UI-M-BH1-alr-f-05-0-UI |
| | | | /clone_end = 3 /gb = AW047083 /gi = 5907612 |
| | | | /ug = Mm.29398 /len = 311 /STRA = rev |
| 137 | Unknown | 2.5 | Cluster Incl AA869278: vq49h11.r1 Mus musculus |
| | | | cDNA, 5 end /clone = IMAGE-1097637 /close_end = 5 |
| | | | /gb = AA869278 /gi = 2964723 /ug = Mm.33918 /len = 366 |
| | | | /STRA = for |
| 138 | Unknown | 2.5 | Cluster Incl AA880158: vv96g12.r1 Mus musculus |
| | | | cDNA, 5 end /clone = IMAGE-1230310 /clone_end = 5 |
| | | | /gb = AA880158 /gi = 2989141 /ug = Mm.86534 /len = 263 |
| | | | /STRA = for |
| 139 | Unknown | 2.5 | Cluster Incl AI448454: mq38g01.x1 Mus musculus |
| | | | cDNA, 3 end /clone = IMAGE-581040 /clone_end = 3 |
| | | | /gb = AI448454 /gi = 4282766 /ug = Mm.32005 /len = 292 |
| | | | /STRA = rev |
| 140 | Unknown | 2.5 | Cluster Incl AI8408I5: UI-M-AH0-adb-c-03-0-UI.s1 Mus |
| | | | musculus cDNA, 3 end /clone = UI-M-AH0-adb-c-03-0- |
| | | | UI /clone_end = 3 /gb = AI840815 /gi = 5475028 |
| | | | /ug = Mm.25269 /len = 363 /STRA = for |
| 141 | Unknown | 2.5 | Cluster Incl AA796759: vp27c02.r1 Mus musculus |
| | | | cDNA, 5 end /clone = IMAGE-1077890 /clone_end = 5 |
| | | | /gb = AA796759 /gi = 2859714 /ug = Mm.29293 /len = 484 |
| | | | /STRA = for |
| 142 | complement factor H-related | 2.5 | Cluster Incl M29008: Mouse complement factor H- |
| | protein | | related protein mRNA, complete cds, clone 13G1 |
| | | | /cds = (64, 1095) /gb = M29008 /gi = 192559 |
| | | | /ug = Mm.37217 /len = 1734 /STRA = for |
| 143 | Unknown | 2.5 | Cluster Incl AI608436: vk2ie08.x1 Mus musculus |
| | | | cDNA, 3 end /clone = IMAGE-947270 /clone_end = 3 |
| | | | /gb = AI608436 /gi = 4617603 /ug = Mm.7031 /len = 496 |
| | | | /STRA = for |
| 144 | Unknown | 2.5 | Cluster Incl AI891912: ul60d10.x1 Mus musculus |
| | | | cDNA, 3 end /clone = IMAGE-2123443 clone_end = 3 |
| | | | /gb = AI891912 /gi = 5597814 /ug = Mm.39726 /len = 496 |
| | | | /STRA = for |
| 145 | poly(A) polymerase VI | 2.5 | Cluster Incl U58134: Mus musculus poly(A) |
| | | | polymerase VI mRNA, complete cds /cds = (149, 1030) |
| | | | /gb = U58134 /gi = 1377869 /ug = Mm.3170 /len 1046 |
| | | | /STRA = for |
| 146 | Unknown | 2.5 | Cluster Incl C80774: C80774 Mus musculus cDNA, 3 |
| | | | end /clone = J0088A04 /clone_end = 3 /gb = C80774 |
| | | | /gi = 2521104 /ug = Mm.25087 /len = 513 /STRA = rev |
| 147 | sid478p | 2.4 | Cluster Incl AB025408: Mus musculus mRNA for |
| | | | sid478p, complete cds /cds = (74, 922) /gb = AB025408 |
| | | | /gi = 5931564 /ug = Mm.1017 /len = 1056 /STRA = for |
| 148 | Unknown | 2.4 | Cluster Incl AW048963: UI-M-BH1-amo-g-04-0-UI.s1 |
| | | | Mus musculus cDNA, 3 end /clone = UI-M-BH1-amo-g- |
| | | | 04-0-UI /clone_end = 3 /gb = AW048963 /gi = 5909492 |
| | | | /ug = Mm.41663 /len = 407 /STRA = rev |
| 149 | Integrin alpha 7 | 2.4 | Cluster Incl L23423: Integrin alpha 7 /cds = (174, 3581) |
| | | | /gb = L23423 /gi = 431415 /ug = Mm.15533 /len = 4010 |
| | | | /STRA = rev |
| 150 | Unknown | 2.4 | Cluster Incl C87638: C87638 Mus musculus cDNA, 3 |
| | | | end /clone = J0248F05 /clone_end = 3 /gb = C87638 |
| | | | /gi = 2919595 /ug = Mm.9776 /len = 521 /STRA = rev |
| 151 | transferrin receptor | 2.4 | Mus musculus /REF = X57349 /DEF = M. musculus |
| | | | mRNA for transferrin receptor /LEN = 2292 (_5, _M, _3 |
| | | | represent transcript regions 5 prime, Middle, and 3 |
| | | | prime respectively) |
| 152 | Procollagen, type I alpha 2 | 2.4 | Cluster Incl X58251: Procollagen, type I, alpha 2 |
| | | | /cds = (79, 4200) /gb = X58251 /gl = 50488 /ug = Mm.4482 |
| | | | /len = 4270 /STRA = for |
| 153 | Secretory protein SEC23 | 2.4 | Cluster Incl D12713: Secretory protein SEC23 related |
| | related | | gene (S. cerevisiae) /cds = (0, 1721) /gb = D12713 |
| | | | /gi = 220493 /ug = Mm.86530 /len = 1722 /STRA = for |
| 154 | Unknown | 2.4 | Cluster Incl AW121776: UI-M-BH2.3-aoe-g-02-0-UI.s1 |
| | | | Mus musculus cDNA, 3 end /clone = UI-M-BH2.3-aoe- |
| | | | g-02-0-UI /clone_end = 3 /gb = AW121776 /gi = 6097126 |
| | | | /ug = Mm.3327 /len = 389 /STRA = for |
| 155 | Unknown | 2.4 | Cluster Incl AW122396: UI-M-BH2.2-aow-d-09-0-UI.s1 |
| | | | Mus musculus cDNA, 3 end /clone = UI-M-BH2.2-aow- |
| | | | d-09-O-UI /clone_end = 3 /gb = AW122396 /gi = 6097897 |
| | | | /ug = Mm.23664 /len = 232 /STRA = rev |
| 156 | Unknown | 2.4 | Cluster Incl AI838681: UI-M-AO0-aca-f-07-0-UI.s7 Mus |
| | | | musculus cDNA, 3 end /clone = UI-M-AO0-aca-f-07-0- |
| | | | UI /clone_end = 3 /gb = A1838681 /gi = 5472894 |
| | | | /ug = Mm.30864 /len = 358 /STRA = for |
| 157 | Unknown | 2.4 | Cluster Incl AI642060: ub75c12.x1 Mus musculus |
| | | | cDNA, 3 end /clone = IMAGE-1383574 /clone_end = 3 |
| | | | /gb = AI642060 /gi = 4720535 /ug = Mm.85459 /len = 505 |
| | | | /STRA = for |
| 158 | Unknown | 2.4 | Cluster Incl AW122448: UI-M-BH2.2-aou-a-04-0-UI.s1 |
| | | | Mus musculus cDNA, 3 end /clone = UI-M-BH2.2-aou- |
| | | | a-04-0-UI /clone_end = 3 /gb = AW122448 /gi = 6097967 |
| | | | /ug = Mm.29770 /len = 217 /STRA = for |
| 159 | IKB kinase beta (IKKbeta) | 2.4 | Cluster Incl AF026524: Mus musculus IKB kinase beta |
| | | | (IKKbeta) mRNA, complete cds /cds = (0, 2273) |
| | | | /gb = AF026524 /gi = 3213210 /ug = Mm.86427 /len = 2274 |
| | | | /STRA = for |
| 160 | Unknown | 2.4 | Cluster Incl AI646301: vs77f11.x1 Mus musculus |
| | | | cDNA, 3 end /clone = IMAGE-1152333 /clone_end = 3 |
| | | | /gb = AI646301 /gi = 4724776 /ug = Mm.3702 /len = 305 |
| | | | /STRA = for |
| 161 | interleukin 1 receptor, type II | 2.4 | Cluster Incl X59769: Interleukin 1 receptor, type II |
| | | | /cds(92, 1324) /gb = X59769 /gi = 52670 /ug = Mm.1349 |
| | | | /len = 1341 /STRA = for |
| 162 | Unknown | 2.4 | Cluster Incl AW208938: uo65g07.x1 Mus musculus |
| | | | cDNA, 3 end /clone = IMAGE-2647452 /clone_end = 3 |
| | | | /gb = AW208938 /gi = 6514878 /ug = Mm.2252 /len = 715 |
| | | | /STRA = for |
| 163 | Unknown | 2.4 | Cluster Incl AI839681: UI-M-ANO-acp-d-02-0-UI.s1 |
| | | | Mus musculus cDNA, 3 end /clone = UI-M-ANO-acp-d- |
| | | | 02-0-UI /clone_end = 3 /gb = AI839681 /gi = 5473894 |
| | | | /ug = Mm.29795 /len = 348 /STRA = for |
| 164 | Unknown | 2.3 | Cluster Incl AI841303: UI-M-AMO-adu-h-03-0-UI.s1 |
| | | | Mus musculus cDNA, 3 end /clone = UI-M-AMO-adu-h- |
| | | | 03-0-UI /clone_end = 3 /gb = AI841303 /gi = 5475516 |
| | | | /ug = Mm.1222 /len = 472 /STRA = for |
| 165 | Unknown | 2.3 | Cluster Incl AI847246: UI-M-AI1-afq-f-01-0-UI.s1 Mus |
| | | | musculus cDNA, 3 end /clone = UI-M-AI1-afq-f-01-0-UI |
| | | | /clone_end = 3 /gb = AI847246 /gi = 5491152 |
| | | | /ug = Mm.2916 /len = 328 /STRA = for |
| 166 | MAD homolog 1 | 2.3 | Cluster Incl U58992: MAD homolog 1 (Drosophila) |
| | | | /cds = (70, 1467) /gb = U58992 /gi = 1518644 |
| | | | /ug = Mm.15185 /len = 1639 /STRA = for |
| 167 | Unknown | 2.3 | Cluster Incl AW125713: UI-M-BH2.2-aql-c-12-0-UI.s1 |
| | | | Mus musculus cDNA, 3 end /clone = UI-M-BH2.2-aql-c- |
| | | | 12-0-UI /clone_end = 3 /gb = AW125713 /gl = 6101283 |
| | | | /ug = Mm.19126 /len = 445 /STRA = for |
| 168 | N-acetylglucosamine-6-O- | 2.3 | Cluster Incl AB011451: Mus musculus mRNA for N- |
| | sulfotransferase | | acetylglucosamine-6-O-sulfotransferase, complete cds |
| | | | /cds = (469, 1920) /gb = AB011451 /gi = 3402294 |
| | | | /ug = Mm.12968 /len = 2150 /STRA for |
| 169 | Unknown | 2.3 | Cluster Incl AW123500: UI-M-BH2.1-aqb-a-12-0-UI.s1 |
| | | | Mus musculus cDNA 3 end /clone = UI-M-BH2.1-aqb-a- |
| | | | 12-D-UI /clone_end = 3 /gb = AW123500 /gi = 6099030 |
| | | | /ug = Mm.80536 /len = 341 /STRA = for |
| 170 | Unknown | 2.3 | Cluster Incl AI847621: UI-M-AP1-agg-e-02-0-UI.s1 |
| | | | Mus musculus cDNA, 3 end /clone = UI-M-AP1-agg-e- |
| | | | 02-0-UI /clone_end = 3 /gb = AI847621 /gi = 5491527 |
| | | | /uq = Mm.41045 /len = 391 /STRArev |
| 171 | Unknown | 2.3 | Cluster Incl AW046525: UI-M-BH1-akz-f-12-0-UI.s1 |
| | | | Mus musculus cDNA, 3 end /clone = UI-M-BH1-akz-f- |
| | | | 12-0-UI /clone_end = 3 /gb = AW046525 /gi = 5907054 |
| | | | /ug = Mm.44651 /len = 612 /STRA = rev |
| 172 | Cortactin | 2.3 | Cluster Incl U03184: Cortactin /cds = (130, 1770) |
| | | | /gb = U03184 /gi = 414990 /ug = Mm.22024 /len = 1867 |
| | | | /STRA = for |
| 173 | osf-2 mRNA for osteoblast | 2.3 | Cluster Incl D13664: Mus musculus osf-2 mRNA for |
| | specific factor 2 | | osteoblast specific factor 2, complete cds |
| | | | /cds = (18, 2453) /gb = D13664 /gi = 393321 |
| | | | /ug = Mm.10681 /len = 3187 /STRA = for |
| 174 | dbpA murine homologue | 2.3 | Cluster Incl D14485: Mouse mRNA for dbpA murine |
| | | | homologue, complete cds /cds = (183, 1109) |
| | | | /gb = D14485 /gi = 1160330 /ug = Mm.29794 /len = 1647 |
| | | | /STRA = for |
| 175 | voltage-gated sodium channel | 2.3 | Cluster Incl L36179: Mouse voltage-gated sodium |
| | | | channel mRNA /cds = (251, 5296) /gb = L36179 |
| | | | /gi = 609544 /ug = Mm.4623 /len = 5482 /STRA = for |
| 176 | Unknown | 2.3 | Cluster Incl AI956230: ul73a07.y1 Mus musculus |
| | | | cDNA, 5 end /clone = IMAGE-2136180 /clone_end = 5 |
| | | | /gb = AI956230 /gi = 5748957 /ug = Mm.21544 /len = 513 |
| | | | /STRA = for |
| 177 | Unknown | 2.3 | Cluster Incl AI642664: vw01e04.x1 Mus musculus |
| | | | cDNA, 3 end /clone = IMAGE-1230654 /clone_end = 3 |
| | | | /gb = AI642664 /gi = 4721139 /ug = Mm.67919 /len = 429 |
| | | | /STRA = for |
| 178 | Unknown | 2.3 | Cluster Incl AA822412: vw37d10.r1 Mus musculus |
| | | | cDNA, 5 end /clone = IMAGE-1246003 /clone_end = 5 |
| | | | /gb = AA822412 /gi = 2892280 /ug = Mm.35596 /len = 460 |
| | | | /STRA = for |
| 179 | Unknown | 2.3 | Cluster Incl AW122633: UI-M-BH2.2-aol-c-12-0-UI.s1 |
| | | | Mus musculus cDNA, 3 end /clone = UI-M-BH2.2-aol-c- |
| | | | 12-0-Ui /clone_end = 3 /gb = AW122633 /gi = 6098161 |
| | | | /ug = Mm.68486 /len = 304 /STRA = for |
| 180 | Unknown | 2.3 | Cluster Incl AW121369: UI-M-BH2.2-aom-e-05-0-UI.s1 |
| | | | Mus musculus cDNA, 3 end /clone = UI-M-BH2.2-aom- |
| | | | e-05-0-UI /clone_end = 3 /gb = AW121369 /gi = 6096702 |
| | | | /ug = Mm.71003 /len = 546 /STRA = rev |
| 181 | Unknown | 2.3 | Cluster Incl AI642662: vw01dO7.x1 Mus musculus |
| | | | cDNA, 3 end /clone = IMAGE-1230637 /clone_end = 3 |
| | | | /gb = AI642662 /gi = 4721137 /ug = Mm.3994 /len = 307 |
| | | | /STRA = for |
| 182 | Unknown | 2.2 | Cluster Incl AA959574: ub54h03.s1 Mus musculus |
| | | | cDNA, 3 end /clone = IMAGE-1381589 /clone_end = 3 |
| | | | /gb = AA959574 /gi = 3125474 /ug = Mm.74711 /len = 309 |
| | | | /STRA = for |
| 183 | Unknown | 2.2 | Cluster Incl AI891576: ul31h02.x1 Mus musculus |
| | | | cDNA, 3 end /clone = IMAGE-2099955 /clone_end = 3 |
| | | | /gb = AI891576 /gi = 5597478 /ug = Mm.43944 /len = 477 |
| | | | /STRA = rev |
| 184 | PACAP ligand precursor | 2.2 | Cluster Incl AB010149: Mus musculus gene for |
| | | | PACAP ligand precursor, complete cds |
| | | | /cds = (248, 775) /gb = AB010149 /gi = 3152419 |
| | | | /ug = Mm.3407 /len = 2418 /STRA = for |
| 185 | la-associated invariant chain | 2.2 | Cluster Incl X00496: Mouse la-associated Invariant |
| | (li) | | chain (li) mRNA fragment /cds = (0, 509) /gb = X00496 |
| | | | /gi = 53102 /ug = Mm.7043 /len = 994 /STRA = for |
| 186 | Unknown | 2.2 | Cluster Incl AI197161: ue51h10.r1 Mus musculus |
| | | | cDNA, 5 end /clone = IMAGE-1494691 /clone_end = 5 |
| | | | /gb = AI197161 /gi = 3749767 /ug = Mm.21288 /len = 475 |
| | | | /STRA = for |
| 187 | Unknown | 2.2 | Cluster Incl AI450295: mt98c05.x1 Mus musculus |
| | | | cDNA, 3 end /clone = IMAGE-637928 /clone_end = 3 |
| | | | /gb = AI450295 /gi = 4296003 /ug = Mm.36136 /len = 384 |
| | | | /STRA = rev |
| 188 | Unknown | 2.2 | Cluster Incl AW049031: UI-M-BH1-amp-g-08-0-UI.s1 |
| | | | Mus musculus cDNA, 3 end /clone = UI-M-BH1-amp-g- |
| | | | 08-0-UI /clone_end = 3 /gb = AW049031 /gi = 5909560 |
| | | | /ug = Mm.34576 /len = 473 /STRA = for |
| 189 | Unknown | 2.2 | Cluster Incl AW125052: UI-M-BH2.1-apx-b-12-0-UI.s1 |
| | | | Mus musculus cDNA, 3 end /clone = UI-M-BH2.1-apx- |
| | | | b-12-0-UI /clone_end3 /gb = AW125052 /gi = 6100582 |
| | | | /ug = Mm.23242 /len = 506 /STRA = rev |
| 190 | Unknown | 2.2 | Cluster Incl AA437598: vd05f08.s1 Mus musculus |
| | | | cDNA, 5 end /clone = IMAGE-791655 /clone_end = 5 |
| | | | /gb = AA437598 /gi = 2142512 /ug = Mm.36496 /len = 410 |
| | | | /STRA = for |
| 191 | D-E-A-D (aspartate- | 2.2 | Cluster Incl U91922: D-E-A-D (aspartate-glutamate- |
| | glutamate-alanine-aspartate) | | alanine-aspartate) box polypeptide 9 /cds = (107, 4249) |
| | box polypeptide 9 | | /gb = U91922 /gi = 2961455 /ug = Mm.20000 /len = 4498 |
| | | | /STRA = for |
| 192 | Unknown | 2.2 | Cluster Incl AI585789: vs07c07.x1 Mus musculus |
| | | | cDNA, 3 end /clone = IMAGE-1137516 /clone_end = 3 |
| | | | /gb = AI585789 /gi = 4571686 /ug = Mm.30473 /len = 370 |
| | | | /STRA = rev |
| 193 | Protein C receptor, | 2.2 | Cluster Incl L39017: Protein C receptor, endothelial |
| | endothelial | | /cds = (55, 783) /gb = L39017 /gi = 728480 /ug = Mm.3243 |
| | | | /len = 1358 /STRA = for |
| 194 | Unknown | 2.2 | Cluster Incl AA795285: vv03h09.r1 Mus musculus |
| | | | cDNA, 5 end /clone = IMAGE-1210625 /clone_end = 5 |
| | | | /gb = AA795285 /gi = 2858240 /ug = Mm.28387 /len = 297 |
| | | | /STRA = rev |
| 195 | mGBP-2 protein | 2.2 | Cluster Incl AJ007970: Mus musculus mRNA for |
| | | | mGBP-2 protein /cds = (163, 1932) /gb = AJ007970 |
| | | | /gi = 4158171 /ug = Mm.24038 /len = 2464 /STRA = for |
| 196 | Mouse chromosome 6 BAC- | 2.2 | Cluster Incl AC002397: Mouse chromosome 6 BAC- |
| | 284H12 | | 284H12 (Research Genetics mouse BAC library) |
| | | | complete sequence /cds = (560, 2197) /gb = AC002397 |
| | | | /gi = 3287367 /ug = Mm.35708 /len = 2778 /STRA = for |
| 197 | Gsg3 gene for actin capping | 2.2 | Cluster Incl AB026984: Mus musculus Gsg3 gene for |
| | protein alpha | | actin capping protein alpha, complete cds |
| | | | /cds = (0, 899) /gb = AB026984 /gi = 5295995 |
| | | | /ug = Mm.12817 /len = 900 /STRA = for |
| 198 | Unknown | 2.1 | Cluster Incl U90123: Hematological and neurological |
| | | | expressed sequence 1 /cds = (95, 559) /gb = U90123 |
| | | | /gi = 1864164 /ug = Mm.1775 /len = 1384 /STRA = for |
| 199 | histone H1 | 2.1 | Cluster Incl J03482: Mouse histone H1 gene, complete |
| | | | cds /cds = (42, 680) /gb = J03482 /gi = 193847 |
| | | | /ug = Mm.22570 /len = 725 /STRA = for |
| 200 | Unknown | 2.1 | Cluster Incl AW046236: UI-M-BH1-ala-b-09-0-UI.s1 |
| | | | Mus musculus cDNA, 3 end /clone = Ui-M-BH1-ala-b- |
| | | | 09-0-UI /clone_end = 3 /gb = AW046236 /gi = 5906765 |
| | | | /ug = Mm.27440 /len = 333 /STRA = for |
| 201 | Unknown | 2.1 | Cluster Incl AA797617: vw26a11.r1 Mus musculus |
| | | | cDNA, 5 end /clone = IMAGE-1244924 /clone_end = 5 |
| | | | /gb = AA797617 /gi = 2860572 /ug = Mm.86584 /len = 252 |
| | | | /STRAfor |
| 202 | NAD-dependent | 2.1 | Cluster Incl J04627: Mouse NAD-dependent |
| | methylenetetrahydrofolate | | methylenetetrahydrofolate dehydrogenase- |
| | dehydrogenase- | | methenyltetrahydrofolate cyclohydrolase mRNA, |
| | methenyltetrahydrofolate | | complete cds /cds = UNKNOWN /gb = J04627 |
| | cyclohydrolase mRNA | | /gi = 200070 /ug = Mm.443 /len2012 /STRA = for |
| 203 | purine nucleoside | 2.1 | Cluster Incl U35374: Mus musculus purine nucleoside |
| | phosphorylase (Np-b) | | phosphorylase (Np-b) mRNA, complete cds |
| | | | /cds = (0, 869) /gb = U35374 /gi = 1144566 /ug = Mm.17932 |
| | | | /len = 870 /STRA = for |
| 204 | Unknown | 2.1 | Cluster Incl AI838379: UI-M-AJ0-aav-a-02-0-UI.s2 Mus |
| | | | musculus cDNA, 3 end /clone = UI-M-AJ0-aav-a-02-0- |
| | | | UI /clone_end = 3 /gb = AI838379 /gi = 5472592 |
| | | | /ug = Mm.27965 /len = 372 /STRA = for |
| 205 | Unknown | 2.1 | Cluster Incl AI842065: UI-M-AN1-afg-a-10-0-UI.s1 Mus |
| | | | musculus cDNA, 3 end /clone = Ui-M-AN1-afg-a-10-0- |
| | | | UI /clone_end = 3 /gb = AI842065 /gi = 5476278 |
| | | | /ug = Mm.24647 /len = 444 /STRA = for |
| 206 | Unknown | 2.1 | Cluster Incl AI849721: UI-M-AL1-ahn-b-07-0-UI.s1 Mus |
| | | | musculus cDNA, 3 end /clone = UI-M-AL1-ahn-b-07-0- |
| | | | UI /clone_end = 3 /gb = A1849721 /gi = 5493627 |
| | | | /ug = Mm.1654 /len = 352 /STRA = for |
| 207 | Unknown | 2.1 | Cluster md AI850834: UI-M-BH0-ajt-a-07-0-UI.s1 Mus |
| | | | musculus cDNA, 3 end /clone = UI-M-BH0-ajt-a-07-0-UI |
| | | | /clone_end = 3 /gb = A1850834 /gi = 5494740 |
| | | | /ug = Mm.40892 /len = 223 /STRA = for |
| 208 | Unknown | 2.1 | Cluster Incl Y07836: M. musculus mRNA for basic- |
| | | | helix-loop-helix protein /cds = (0, 1214) /gb = Y07836 |
| | | | /gi = 1707584 /ug = Mm.2436 /len = 1508 /STRA = for |
| 209 | Xeroderma pigmentosum, | 2.1 | Cluster Incl U27398 Xeroderma pigmentosum, |
| | complementation group C | | complementation group C /cds = (90, 2792) |
| | | | /gb = U27398 /gi = 1276472 /ug = Mm.2806 /len = 3031 |
| | | | /STRA = for |
| 210 | Unknown | 2.1 | Cluster Incl AW046036: UI-M-BH1-akv-b-01-0-UI.s1 |
| | | | Mus musculus cDNA, 3 end /clone = UI-M-BH1-akv-b- |
| | | | 01-0-UI /clone_end = 3 /gb = AW046036 /gi = 5906565 |
| | | | /ug = Mm.25849 /len = 289 /STRA = for |
| 211 | Msx-interacting-zinc finger | 2.1 | Cluster Incl AF039567: Mus musculus Msx-interacting- |
| | protein 1 (Miz1) | | zinc finger protein 1 (Miz1) mRNA, complete cds |
| | | | /cds = (26, 1498) /gb = AF039567 /gi = 2773147 |
| | | | /ug = Mm.6370 /len = 1847 /STRA = for |
| 212 | Unknown | 2.0 | Cluster Incl AW060474: UI-M-BH1-anj-h-09-0-UI.s1 |
| | | | Mus musculus cDNA, 3 end /clone = UI-M-BH1-anj-h- |
| | | | 09-0-UI /clone_end = 3 /gb = AW060474 /gi = 6008225 |
| | | | /ug = Mm.39328 /len = 405 /STRA = rev |
| 213 | Unknown | 2.0 | Cluster Incl AI852289: UI-M-BH0-aje-e-03-0-UI.s1 Mus |
| | | | musculus cDNA, 3 end /clone = UI-M-BHO-aje-e-03-0- |
| | | | UI /clone_end = 3 /gb = AI852289 /gi = 5496195 |
| | | | /ug = Mm.30666 /len = 416 /STRA = rev |
| 214 | Unknown | 2.0 | Cluster Incl AI847028: UI-M-AP1-agj-f-12-0-UI.s1 Mus |
| | | | musculus cDNA, 3 end /clone = UI-M-AP1-agj-f-12-0-UI |
| | | | /clone_end = 3 /gb = AI847028 /gi = 5490934 |
| | | | /ug = Mm.35083 /len = 307 /STRA = for |
| 215 | transaldolase | 2.0 | Cluster Incl U67611: Mouse transaldolase gene |
| | | | mRNA, complete cds /cds = (5, 1018) /gb = U67611 |
| | | | /gi = 2589165 /ug = Mm.29182 /len = 1155 /STRA = for |
| 216 | Unknown | 2.0 | Cluster Incl AI839392: UI-M-AN0-aco-b-02-0-UI.s1 |
| | | | Mus musculus cDNA, 3 end /clone = UI-M-AN0-aco-b- |
| | | | 02-0-UI /clone_end = 3 /gb = A1839392 /gi = 5473605 |
| | | | /ug = Mm.24174 /len = 181 /STRA = for |
| 217 | Unknown | 2.0 | Cluster Incl AI747699: ul21b07.x1 Mus musculus |
| | | | cDNA, 3 end /clone = IMAGE-2088181 /clone_end = 3 |
| | | | /gb = AI747699 /gi = 5125963 /ug = Mm.30678 /len = 542 |
| | | | /STRA = for |
| 218 | NDPP-1 | 2.0 | Cluster Incl D10727: Mus musculus mRNA for NDPP-1 |
| | | | protein, complete cds /cds = (857, 2026) /gb = D10727 |
| | | | /gi = 220499 /ug = Mm.4941 /len = 4308 /STRA = rev |
| 219 | Unknown | 2.0 | Cluster Incl AI847104: UI-M-AP1-agn-f-09-0-UI.s1 Mus |
| | | | musculus cDNA, 3 end /clone = UI-M-AP1-agn-f-09-0- |
| | | | UI /clone_end = 3 /gb = AI847104 /gi = 5491010 |
| | | | /ug = Mm.22856 /len = 304 /STRA = for |
| 220 | 14-3-3 protein gamma | 2.0 | Cluster Incl AF058799: Mus musculus 14-3-3 protein |
| | | | gamma mRNA, complete cds /cds = (182, 925) |
| | | | /gb = AF058799 /gi = 3065928 /ug = Mm.10802 /len = 1134 |
| | | | /STRA = for |
| 221 | serine protease | 2.0 | Cluster Incl D89871: Mus musculus mRNA for serine |
| | | | protease, complete cds /cds = (244, 2529) /gb = D89871 |
| | | | /gi = 2696702 /ug = Mm.9431 /len = 2614 /STRA = for |
| 222 | Unknown | 2.0 | Cluster Incl AI852555: UI-M-BH0-aiw-g-04-0-UI.s1 Mus |
| | | | musculus cDNA, 3 end /clone = UI-M-BH0-aiw-g-04-0- |
| | | | UI /clone_end = 3 /gb = AI852555 /gi = 5496461 |
| | | | /ug = Mm.7780 /len = 351 /STRA = for |
| 223 | Calcium/calmodulin- | 2.0 | Cluster Incl AF059029: Mus musculus |
| | dependent protein kinase II | | calcium/calmodulin-dependent protein kinase II delta |
| | delta | | mRNA, partial cds /cds = (0, 523) /gb = AF059029 |
| | | | /gi = 3088550 /ug = Mm.57082 /len = 524 /STRA = for |
| 224 | B-IND1 protein (B-ind1 gene) | 2.0 | Cluster Incl Z97207: Mus musculus partial mRNA for |
| | | | B-IND1 protein (B-ind1 gene) /cds = (0, 570) |
| | | | /gb = Z97207 /gi = 5777593 /ug = Mm.57023 /len = 571 |
| | | | /STRA = for |
| 225 | Unknown | 2.0 | Cluster Incl AI594658: vt53a07.x1 Mus musculus |
| | | | cDNA, 3 end /cloneIMAGE-1166772 /clone_end = 3 |
| | | | /gb = AI594658 /gi = 4603706 /ug = Mm.52258 /len = 178 |
| | | | /STRA = for |
| 226 | Unknown | 2.0 | Cluster Incl AI891565: ul31g01.x1 Mus musculus |
| | | | CDNA, 3 end /clone = IMAGE-2099952 /clone_end = 3 |
| | | | /gb = AI891565 /gi = 5597467 /ug = Mm.40617 /len = 487 |
| | | | /STRA = rev |
| 227 | Unknown | 2.0 | Cluster Incl AW120812: UI-M-BH2.3-aoa-b-05-0-UI.s1 |
| | | | Mus musculus cDNA, 3 end /clone = UI-M-BH2.3-aoa- |
| | | | b-05-0-UI /clone_end = 3 /gb = AW120812 /gi = 6096145 |
| | | | /ug = Mm.76705 /len = 133 /STRA = rev |
| 228 | NAD(P)H menadione | −2.0 | Cluster Incl UI12961: NAD(P)H menadione |
| | oxidoreductase 1, dioxin | | oxidoreductase 1, dioxin inducible /cds = (108, 932) |
| | inducible | | /gb = U12961 /gi = 595517 /ug = Mm.252 /len = 1528 |
| | | | /STRA = for |
| 229 | Unknown | −2.0 | Cluster Incl AI881990: ul56h10.x1 Mus musculus |
| | | | cDNA, 3 end /clone = IMAGE-2123107 /clone_end = 3 |
| | | | /gb = AI881990 /gi = 5567142 /ug = Mm.21211 /len = 513 |
| | | | /STRA = rev |
| 230 | Unknown | −2.0 | Cluster Incl AA175250: ms86g06.r1 Mus musculus |
| | | | cDNA, 5 end /clone = IMAGE-618490 /clone_end = 5 |
| | | | /gb = AA175250 /gi = 1756501 /ug = Mm.26809 /len = 396 |
| | | | /STRA = rev |
| 231 | Unknown | −2.0 | Escherichia coli /REF = J04423 /DEF = E coli bioB gene |
| | | | biotin synthetase /LEN1114 (−5, −M, −3 represent |
| | | | transcript regions 5 prime, Middle, and 3 prime |
| | | | respectively) |
| 232 | Unknown | −2.0 | Escherichia coli /REF = J04423 /DEF = E coli bioB gene |
| | | | biotin synthetase /LEN = 1114 (−5, −M, −3 represent |
| | | | transcript regions 5 prime, Middle, and 3 prime |
| | | | respectively) |
| 233 | Tubulin alpha1 | −2.0 | Cluster Incl M28729: Tubulin alpha1 /cds = (0, 596) |
| | | | /gb = M28729 /gi = 202222 /ug = Mm.88110 /len = 786 |
| | | | /STRA = for |
| 234 | Unknown | −2.1 | Cluster Incl AI510157: vy01cO5.y1 Mus musculus |
| | | | cDNA, 5 end /clone = IMAGE-1294184 /clone_end = 5 |
| | | | /gb = AI510157 /gi = 4409062 /ug = Mm.21630 /len = 271 |
| | | | /STRA = for |
| 235 | Unknown | −2.1 | Cluster Incl AI464754: mz76c08.y1 Mus musculus |
| | | | cDNA, 5 end /clone = IMAGE-719342 /clone_end = 5 |
| | | | /gb = AI464754 /gi = 4318784 /ug = Mm.38163 /len = 486 |
| | | | /STRA = rev |
| 236 | Unknown | −2.1 | Escherichia coli /REF = J04423 /DEF = E coli bioB gene |
| | | | biotin synthetase /LEN = 1114 (−5, −M, −3 represent |
| | | | transcript regions 5 prime, Middle, and 3 prime |
| | | | respectively) |
| 237 | Unknown | −2.1 | Escherichia coli /REF = J04423 /DEF = E coli bioB gene |
| | | | biotin synthetase /LEN = 1114 (−5, −M, −3 represent |
| | | | transcript regions 5 prime, Middle, and 3 prime |
| | | | respectively) |
| 238 | Unknown | −2.1 | Escherichia coli /REF = J04423 /DEF = E coli bioB gene |
| | | | biotin synthetase /LEN = 1114 (−5, −M, −3 represent |
| | | | transcript regions 5 prime, Middle, and 3 prime |
| | | | respectively) |
| 239 | Unknown | −2.1 | Escherichia coli /REF = J04423 /DEF = E coli bioB gene |
| | | | biotin synthetase /LEN = 1114 (−5, −M, −3 represent |
| | | | transcript regions 5 prime, Middle, and 3 prime |
| | | | respectively) |
| 240 | Unknown | −2.2 | Cluster Incl AW046853: UI-M-BH1-alz-h-12-0-UI.s1 |
| | | | Mus musculus cDNA, 3 end /clone = UI-M-BH1-alz-h- |
| | | | 12-0-UI /clone_end = 3 /gb = AW046853 /gi = 5907382 |
| | | | /ug = Mm.44740 /len = 390 /STRA = rev |
| 241 | Unknown | −2.2 | Cluster Incl AA968017: uh06h10.r1 Mus musculus |
| | | | cDNA, 5 end /clone = IMAGE-1617187 /clone_end = 5 |
| | | | /gb = AA968017 /gi = 3141910 /ug = Mm.29397 /len = 409 |
| | | | /STRA = for |
| 242 | Unknown | −2.2 | Cluster Incl AW2I2541: uo0O8d11.x1 Mus musculus |
| | | | cDNA, 3 end /clone = IMAGE-2598933 /clone_end = 3 |
| | | | /gb = AW212541 /gi = 6518638 /ug = Mm.89634 /len = 704 |
| | | | /STRA = for |
| 243 | Placental growth factor | −2.2 | Cluster Incl X80171: Placental growth factor |
| | | | /cds = (118, 594) /gb = X80171 /gi = 1063401 |
| | | | /ug = Mm.4809 /len = 1370 /STRA = for |
| 244 | ATP receptor | −2.2 | Cluster Incl X84896: M. musculus mRNA for ATP |
| | | | receptor /cds = (164, 1363) /gb = X84896 /gi = 1166454 |
| | | | /ug = Mm.25722 /len = 1760 /STRA = for |
| 245 | Unknown | −2.2 | Cluster Incl AI841064: UI-M-AMO-ado-c-06-0-UI.s1 |
| | | | Mus musculus cDNA, 3 end /clone = UI-M-AMO-ado-c- |
| | | | 06-0-UI /clone_end = 3 /gb = AI841064 /gi = 5475242 |
| | | | /ug = Mm.39349 /len = 374 /STRA = rev |
| 246 | Actin, beta, cytoplasmic | −2.2 | Cluster Incl M12481: Actin, beta, cytoplasmic |
| | | | /cds = (0, 1049) /gb = M12481 /gi = 191581 |
| | | | /ug = Mm.89919 /len = 1109 /STRA = for |
| 247 | Unknown | −2.3 | Cluster Incl AA763515: vp57g11.r1 Mus musculus |
| | | | cDNA, 3 end /clone = IMAGE-1080836 /clone_end = 3 |
| | | | /gb = AA763515 /gi = 2813262 /ug = Mm.26097 /len = 544 |
| | | | /STRA = rev |
| 248 | Unknown | −2.3 | Cluster Incl AI846515: UI-M-AN1-aff-d-11-0-UI.s1 Mus |
| | | | musculus cDNA, 3 end /clone = UI-M-AN1-aff-d-11-0-UI |
| | | | /clone_end = 3 /gb = AI846515 /gi = 5490421 |
| | | | /ug = Mm.41465 /len = 368 /STRA = for |
| 249 | Unknown | −2.3 | Cluster Incl AI586002: vs37f11.x1 Mus musculus |
| | | | cDNA, 3 end /clone = IMAGE-1148493 /clone_end3 |
| | | | gb = AI586002 /gi = 4571899 /ug = Mm.36721 /len = 446 |
| | | | /STRA = for |
| 250 | ribosomal protein L41 | −2.3 | Cluster Incl U93862: Mus musculus ribosomal protein |
| | | | L41 mRNA, complete cds /cds = (15, 92) /gb = U93862 |
| | | | /gi = 1938401 /ug = Mm.13859 /len = 201 /STRA = for |
| 251 | Unknown | −2.4 | Cluster Incl AA867818: vx18c05.r1 Mus musculus |
| | | | cDNA, 5 end /clone = IMAGE-1264808 /clone_end = 5 |
| | | | /gb = AA867818 /gi = 2963263 /ug = Mm.24368 /len = 420 |
| | | | /STRA = for |
| 252 | Deubiquitinating enzyme 1 | −2.4 | Cluster Incl U41636: Deubiquitinating enzyme 1 |
| | | | /cds(183, 1763) /gb = U41636 /gi = 1302629 |
| | | | /ug = Mm.14130 /len = 2674 /STRA = for |
| 253 | Tubulin alpha1 | −2.5 | Cluster Incl M28729: Tubulin alpha1 /cds = (0, 596) |
| | | | /gb = M28729 /gi = 202222 /ug = Mm.88110 /len = 786 |
| | | | /STRA = for |
| 254 | ubiquitin | −2.5 | Cluster Incl X51703: Mouse mRNA for ubiquitin |
| | | | /cds = (104, 1021) /gb = X51703 /gi = 55117 /ug = Mm.235 |
| | | | /len = 1172 /STRA = for |
| 255 | Unknown | −2.6 | Cluster Incl AI549907: vw9Bh11.x1 Mus musculus |
| | | | cDNA, 3 end /clone = IMAGE-1263045 /clone_end = 3 |
| | | | /gb = AI549907 /gi = 4482270 /ug = Mm.34850 /len = 416 |
| | | | /STRA = for |
| 256 | Unknown | −2.6 | Cluster Incl AI551253: vo68c10.x1 Mus musculus |
| | | | cDNA, 3 end /clone = IMAGE-1055058 /clone_end = 3 |
| | | | /gb = A1551253 /gi = 4483616 /ug = Mm.35239 /len = 380 |
| | | | /STRA = rev |
| 257 | A-X actin | −2.6 | Cluster Incl J04181: Mouse A-X actin mRNA, complete |
| | | | cds /cds = (62, 1189) /gb = J04181 /gi = 191660 |
| | | | /ug = Mm.297 /len = 1857 /STRA = for |
| 258 | Unknown | −2.7 | Cluster Incl AI877190: uc56g08.r1 Mus musculus |
| | | | cDNA, 5 end /clone = IMAGE-1429694 /clone_end = 5 |
| | | | /gb = AI877190 /gl = 5551239 /ug = Mm.87002 /len = 334 |
| | | | /STRA = for |
| 259 | Unknown | −2.8 | Cluster Incl AI849493: UI-M-AH1-agt-d-02-0-UI.s1 Mus |
| | | | musculus cDNA, 3 end /clone = UI-M-AH1-agt-d-02-0- |
| | | | UI /clone_end = 3 /gb = A1849493 /gi = 5493399 |
| | | | /ug = Mm.28693 /len = 377 /STRA = for |
| 260 | Unknown | −2.8 | Cluster Incl AW124340: UI-M-BH2.1-apq-f-11-0-UI.s1 |
| | | | Mus musculus cDNA, 3 end /clone = UI-M-BH2.l-apq-f- |
| | | | 11-0-UI /clone_end = 3 /gb = AW124340 /gi = 6099870 |
| | | | /ug = Mm.30239 /len = 224 /STRA = for |
| 261 | Tumor protein D52-like 1 | −2.8 | Cluster Incl AF004428: Tumor protein D52-like 1 |
| | | | /cds(191, 805) /gb = AF004428 /gi = 2895080 |
| | | | /ug = Mm.7821 /len = 1267 /STRA = for |
| 262 | Unknown | −2.8 | Cluster Incl AI746295: uI05b02.x1 Mus musculus |
| | | | cDNA, 3 end /clone = IMAGE-2065131 /clone_end = 3 |
| | | | /gb = AI746295 /gi = 5124559 /ug = Mm.26684 /len = 560 |
| | | | /STRA = for |
| 263 | Unknown | −2.8 | Cluster Incl AI847278: UI-M-AN1-afh-a-08-0-UI.s1 Mus |
| | | | musculus cDNA, 3 end /clone = UI-M-AN1-afh-a-08-0- |
| | | | UI /clone_end = 3 /gb = AI847278 /gi = 5491184 |
| | | | /ug = Mm.41834 /len = 190 /STRA = rev |
| 264 | Unknown | −2.8 | Cluster Incl AI414611: ma56g02.x1 Mus musculus |
| | | | cDNA, 3 end /clone = IMAGE-314738 /clone_end = 3 |
| | | | /gb = AI414611 /gi = 4258115 /ug = Mm.22943 /len = 379 |
| | | | /STRA = rev |
| 265 | Unknown | −3.0 | Cluster Incl AA474655: vd53b01.s1 Mus musculus |
| | | | cDNA, 5 end /clone = IMAGE-804265 /clone_end = 5 |
| | | | /gb = AA474655 /gi = 2202810 /lug = Mm.38051 /len = 305 |
| | | | /STRA = for |
| 266 | Unknown | −3.0 | Cluster Incl AI841410: UI-M-AH0-acx-b-06-0-UI.s1 Mus |
| | | | musculus cDNA, 3 end /clone = UI-M-AH0-acx-b-06-0- |
| | | | UI /clone_end3 /gb = AI841410 /gi = 5475623 |
| | | | /ug = Mm.30069 /len = 286 /STRA = for |
| 267 | Murine H3.1 gene for histone | −3.1 | Cluster Incl X16496: Murine H3.1 gene for histone |
| | H3.1 | | H3.1 /cds = 32(0, 410) /gb = X16496 /gi = 51326 |
| | | | /ug = Mm.57120 /len = 411 /STRA = for |
| 268 | Mouse rearranged T-cell | −3.1 | Cluster Incl X03278: Mouse rearranged T-cell receptor |
| | receptor beta V14/D1.1/J2.3 | | beta V14/D1.1/J2.3 gene segment /cds = (0, 406) |
| | gene segment | | /gb = X03278 /gi = 54680 /ug = Mm.57190 /len = 407 |
| | | | /STRA-for |
| 269 | Unknown | −3.2 | Cluster Incl AA138611: mr62a12.r1 Mus musculus |
| | | | cDNA, 5 end /clone = IMAGE-602014 /clone_end = 5 |
| | | | /gb = AA138611 /gi = 1700832 /ug = Mm.45237 /len = 424 |
| | | | /STRA = for |
| 270 | Unknown | −3.2 | Cluster Incl AI854034: UI-M-BH0-ajm-c-11-0-UI.s1 |
| | | | Mus musculus cDNA, 3 end /clone = UI-M-BH0-ajm-c- |
| | | | 11-0-UI /clone_end = 3 /gb = AI854034 /gi = 5497940 |
| | | | /ug = Mm.31856 /len = 534 /STRA = rev |
| 271 | Unknown | −3.2 | Cluster Incl AW123974: UI-M-BH2.3-aqc-f-06-0-UI.s1 |
| | | | Mus musculus cDNA, 3 end /clone = UI-M-BH2.3-aqc-f- |
| | | | 06-0-UI /clone_end = 3 /gb = AW123974 /gi = 6099504 |
| | | | /ug = Mm.76760 /len = 370 /STRA = rev |
| 272 | Unknown | −3.2 | Cluster Incl AI876420: uj57a10.x1 Mus musculus |
| | | | cDNA, 3 end /clone = IMAGE-1924026 /clone_end = 3 |
| | | | /gb = AI876420 /gi = 5550469 /ug = Mm.38875 /len = 559 |
| | | | /STRA = rev |
| 273 | Unknown | −3.3 | Cluster Incll AI661736: va30h03.x1 Mus musculus |
| | | | cDNA, 3 end /clone = IMAGE-732917 /clone_end = 3 |
| | | | /gb = AI661736 /gi = 4765319 /ug = Mm.37714 /len = 304 |
| | | | /STRA = rev |
| 274 | Unknown | −3.3 | Cluster Incl AI450812: ms82f10.x1 Mus musculus |
| | | | cDNA, 3 end /clone = IMAGE-618091 /clone_end = 3 |
| | | | /gb = AI450812 /gi = 4299619 /ug = Mm.32362 /len = 477 |
| | | | /STRA = rev |
| 275 | Unknown | −3.5 | Cluster Incl AA616199: vo96g08.r1 Mus musculus |
| | | | cDNA, 5 end /clone = IMAGE-1067006 /clone_end = 5 |
| | | | /gb = AA616199 /gi = 2503404 /ug = Mm.38773 /len = 338 |
| | | | /STRA = rev |
| 276 | Unknown | −3.8 | Cluster Incl AI606844: vw17b12.x1 Mus musculus |
| | | | cDNA, 3 end /clone = IMAGE-1244063 /clone_end = 3 |
| | | | /gb = AI606844 /gi = 4616011 /ug = Mm.37875 /len = 437 |
| | | | /STRA = rev |
| 277 | Unknown | −3.8 | Cluster Incl AW124886: UI-M-BH2.1-apu-c-08-0-UI.s1 |
| | | | Mus musculus cDNA, 3 end /clone = UI-M-BH2.1-apu- |
| | | | c-08-0-UI /clone_end = 3 /gb = AW124886 /gi = 6100416 |
| | | | /ug = Mm.44462 /len = 415 /STRA = rev |
| 278 | Mus musculus EWS/FLI1 | −3.9 | Cluster Incl AF020263: Mus musculus EWS/FLI1 |
| | activated transcript 2 (EAT-2 | | activated transcript 2 (EAT-2) mRNA, complete cds |
| | | | /cds = (0, 398) /gb = AF020263 /gi = 2429353 |
| | | | /ug = Mm.57009 /len = 399 /STRA = for |
| 279 | Unknown | −4.0 | Bacillus subtilis /REF = L38424 /DEF = B subtilis dapB, |
| | | | jojF, jojG genes corresponding to nucleotides 1358-3197 |
| | | | of L38424 /LEN = 1931 (−5, −M, −3 represent |
| | | | transcript regions 5 prime, Middle, and 3 prime |
| | | | respectively) |
| 280 | Unknown | −5.2 | Cluster Incl AI834969: UI-M-AM1-afw-d-05-0-UI.s1 |
| | | | Mus musculus cDNA, 3 end /clone = UI-M-AM1-afw-d- |
| | | | 05-0-UI /clone_end = 3 /gb = AI834969 /gi = 5469182 |
| | | | /ug = Mm.40735 /len = 417 /STRA = rev |
| 281 | Unknown | −5.6 | Cluster Incl AW061035: UI-M-BH1-amn-h-08-0-UI.s1 |
| | | | Mus musculus cDNA, 3 end /clone = UI-M-BH1-amn-h- |
| | | | 08-0-UI /clone_end = 3 /gb = AW61035 /gi = 6008786 |
| | | | /ug = Mm.48651 /len = 460 /STRA = for |

Two hundred eighty-six RAGs were identified in these studies. Some RAGs were upregulated while other RAGs were downregulated. Among the upregulated RAGs, five had previously been reported to be induced by peripheral nerve transection (Wakisaka et al., 1991; Zhang et al., 1996; Mattsson et al., 1998; Laundry et al., 2000; Tsujino et al., 2000), supporting the validity of this method.

Two RAG mRNAs were downregulated more than or equal to five-fold change. Thirty-six RAG mRNAS were upregulated more than or equal to five-fold change. The RAGs of the present invention represented many molecular classes of molecules. For example, 36 genes that were upregulated more than or equal to five-fold change segregated into several categories: neuropeptides (neuropeptide Y, galanin), immune system-related (complement Clq, histocompatibility class 2 antigen), transcription factors (ATF-3, SOX11), cellular metabolism (lysozyme P, glucosaminyltransferase, lysozyme M), cytoskeletal (myosin-X), and others. Both of the downregulated genes were unknown EST clones.

B. RAG Polypeptides

The present invention provides RAG compounds are that are RAG polypeptides or functional analogs thereof, i.e., compounds that functionally mimic polypeptides that promote the regeneration of nerve tissue, e.g., peripheral nerves. Such compounds are suitable for administration to a subject where it is desirable, for example, to promote the growth or differentiation of cells and tissues in the subject, such as nerve cells of either the peripheral nervous system, or the central nervous system. In contrast, pathological conditions typified by excessive neuronal growth or differentiation can be associated with aberrant nerve growth factor activity, brain cancer, and neuroblastoma. Accordingly, it is further an object of the invention to provide for compounds that are functional antagonists of RAG polypeptides. It is also an object of the invention to provide for compounds that are partial antagonists and partial agonists of RAG polypeptides. The compounds of the present invention can be used to treat both acute and chronic neuronal damage, e.g., brain and spinal cord trauma, stroke, cerebral palsy, multiple sclerosis, Parkinson's disease, Alzheimer's disease, ALS, stroke and peripheral neuropathies. The compounds of the present invention are also useful to promote nerve growth following organ transplantation, e.g., heart transplantation surgery.

The invention further relates to structure-based methods useful in identifying, designing and producing compounds which act as functional modulators of both RAGs and RAG polypeptides function.

The present compounds include RAG polypeptides, identified as RAG NOs: 1-281 as detailed in Table 1. Variants, analogs, homologs, or fragments of these compounds, such as spedes homologs, are also included in the present invention, as well as degenerate forms thereof. The present invention also includes small molecule RAG compounds that are RAG mimetics, RAG agonists or RAG antagonists. The RAG polypeptides of the present invention may be capped on the N-terminus or the C-terminus or on both the N-terminus and the C-terminus. The RAG polypeptides of the present invention may be pegylated, or modified, e.g., branching, at any amino acid residue containing a reactive side chain, e.g., lysine residue. The RAG polypeptides of the present invention may be linear or cyclized.

The RAG compounds can contain natural amino acids, non-natural amino acids, d-amino acids and I-amino acids, and any combinations thereof. In certain embodiments, the compounds of the invention can include commonly encountered amino acids which are not genetically encoded. These non-genetically encoded amino acids include, but are not limited to, alanine (*Ala) and other omega-amino acids such as 3-aminopropionic acid (Dap), 2,3-diaminopropionic add (Dpr), 4-aminobutyric acid and so forth; α-aminoisobutyric acid (Aib); ε-aminoheanoic acid (Aha); δ-aminovaleric acid (Ava); N-methylglycine or sarcosine (MeGly); omithine (Om); citrulline (Cit); t-butylalanine (t-BuA); t-butylglycine (t-BuG); N-methylisoleucine (Melle); phenylglycine (Phg); cyclohexylalanine (Cha); norleucine (Nle); 2-naphthylalanine (2-Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic add (Tic); f2-thienylalanine (Thi); methionine sulfoxide (MSO); homoarginine (hArg); N-acetyl lysine (AcLys); 2,3-diaminobutyric acid (Dab); 2,3-diaminobutyric acid (Dbu); p-aminophenylalanine (Phe(pNH₂)); N-methyl valine (MeVal); homocysteine (hCys) and homoserine (hSer). Non-naturally occurring variants of the compounds may be produced by mutagenesis techniques or by direct synthesis. In one aspect of the present invention, the RAG compounds of the invention are prodrugs, ie., the biological activity of the RAG compound is altered, e.g., increased, upon contacting a biological system in vivo or in vitro.

In one embodiment, a RAG compound includes an analog or homolog of RAG ID NOs:1-281. Compounds of the present invention include those with homology to RAG ID NOs:1-281, for example, preferably 50% or greater amino acid Identity, more preferably 75% or greater amino acid Identity, and even more preferably 90% or greater amino acid Identity.

Sequence identity can be measured using sequence analysis software (Sequence Analysis Software Package of the Genetics Computer Group, University of Wisconsin Biotechnology Center, 1710 University Avenue, Madison, Wis. 53705), with the default parameters therein in the case of polypeptide sequences, which are less than 100% identical to a reference sequence, the non-identical positions are preferably, but not necessarily, conservative substitutions for the reference sequence. Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine. Thus, included in the invention are peptides having mutated sequences such that they remain homologous, e.g., in sequence, in structure, in function, and in antigenic character or other function, with a polypeptide having the corresponding parent sequence. Such mutations can, for example, be mutations involving conservative amino acid changes, e.g., changes between amino acids of broadly similar molecular properties. For example, interchanges within the aliphatic group alanine, valine, leucine and isoleucine can be considered as conservative. Sometimes substitution of glycine for one of these can also be considered conservative. Other conservative interchanges include those within the aliphatic group aspartate and glutamate; within the amide group asparagine and glutamine; within the hydroxyl group serine and threonine; within the aromatic group phenylalanine, tyrosine and tryptophan; within the basic group lysine, arginine and histidine; and within the sulfur-containing group methionine and cysteine. Sometimes substitution within the group methionine and leucine can also be considered conservative. Preferred conservative substitution groups are aspartateglutamate; asparagine-glutamine; valine-leudne-Isoleucine; alanine-valine; phenylalanine- tyrosine; and Iysine-arginine.

The invention also provides for compounds having altered sequences including insertions such that the overall amino acid sequence Is lengthened, while the compound still retains the appropriate RAG agonist or antagonist properties. Additionally, altered sequences may include random or designed internal deletions that truncate the overall amino acid sequence of the compounds; however the compound still retains its RAG-III functional properties. In certain embodiments, one or more amino acid residues within RAG ID NOs:1-281 are replaced with other amino acid residues having physical and/or chemical properties similar to the residues they are replacing. Preferably, conservative amino acid substitutions are those wherein an amino acid Is replaced with another amino acid encompassed within the same designated class, as will be described more thoroughly below. Insertions, deletions, and substitutions are appropriate where they do not abrogate the functional properties of the compound. Functionality of the altered compound can be assayed according to the in vitro and in vivo assays described below that are designed to assess the RAG-like properties of the altered compound.

The amino add residues of RAG ID NOs:1-281, analogs or homologs of RAG ID NOs:1-281 include genetically-encoded I-amino adds, naturally occurring non-genetically encoded I-amino acids, synthetic d-amino acids, or d-enantiomers of all of the above

B. RAG Nucleic Acid Sequences

The compounds of the present invention include one or more polynucleotides encoding RAG ID NOs:1-281, including degenerate variants thereof. Accordingly, nucleic acid sequences capable of hybridizing at low stringency with any nucleic acid sequences encoding RAG ID NOs:1-281 are considered to be within the scope of the invention. For example, for a nucleic acid sequence of about 20-40 bases, a typical prehybridization, hybridization, and wash protocol is as follows: (1) prehybridization: incubate nitrocellulose filters containing the denatured target DNA for 3-4 hours at 55° C. in 5× Denhardt's solution, 6×SSC (20×SSC consists of 175 g NaCl, 88.2 g sodium citrate in 800 ml H₂O adjusted to pH 7.0 with 10 N NaOH), 0.1% SDS, and 100 μg/ml denatured salmon sperm DNA, (2) hybridization: incubate filters in prehybridization solution plus probe at 42° C. for 14-48 hours, (3) wash; three 15 minutes washes in 6×SSC and 0.1% SDS at room temperature, followed by a final 1-1.5 minutes wash in 6×SSC and 0.1% SDS at 55° C. Other equivalent procedures, e.g., employing organic solvents such as formamide, are well known in the art.

The invention also encompasses allelic variants of the same, that is, naturally-occurring alternative forms of the isolated polynucleotides that encode polypeptides that are identical, homologous or related to those encoded by the polynucleotides. Alternatively, non-naturally occurring variants may be produced by mutagenesis techniques or by direct synthesis techniques well known in the art.

C. RAG Recombinant Expression Vectors

Another aspect of the invention includes vectors containing one or more nucleic acid sequences encoding a RAG polypeptide. For recombinant expression of one or more the polypeptides of the invention, the nucleic acid containing all or a portion of the nucleotide sequence encoding the polypeptide is inserted into an appropriate cloning vector, or an expression vector (ie., a vector that contains the necessary elements for the transcription and translation of the inserted polypeptide coding sequence) by recombinant DNA techniques well known in the art and as detailed below.

In general, expression vectors useful in recombinant DNA techniques are often in the form of plasmids. In the present specification, “plasmid” and “vector” can be used interchangeably as the plasmid is the most commonly used form of vector. However, the invention Is intended to include such other forms of expression vectors that are not technically plasmids, such as viral vectors (e.g., replication defective retroviruses, adenoviruses and adeno-associated viruses), which serve equivalent functions. Such viral vectors permit infection of a subject and expression in that subject of a compound.

The recombinant expression vectors of the invention comprise a nucleic acid encoding a compound with RAG-like properties in a form suitable for expression of the nucleic acid in a host cell, which means that the recombinant expression vectors include one or more regulatory sequences, selected on the basis of the host cells to be used for expression that is operatively-linked to the nucleic acid sequence to be expressed. Within a recombinant expression vector, “operably-linked” is intended to mean that the nucleotide sequence of interest Is linked to the regulatory sequence(s) in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., Issue-specific regulatory sequences). It will be appreciated by those skilled in the art that the design of the expression vector can depend on such factors as the choice of the host cell to be transformed, the level of expression of polypeptide desired, etc. The expression vectors of the invention can be introduced into host cells to thereby produce polypeptides or peptides, including fusion polypeptides, encoded by nucleic acids as described herein (e.g., RAGs and RAG-derived fusion polypeptides, etc.).

D. RAG-Expressing Host Cells

Another aspect of the invention pertains to RAG-expressing host cells, which contain a nucleic acid encoding one or more RAGs. The recombinant expression vectors of the invention can be designed for expression of RAGs in prokaryotic or eukaryotic cells. For example, RAGs can be expressed in bacterial cells such as Escherichia coli, insect cells (using baculovirus expression vectors), fungal cells, e.g., yeast, yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase. Expression of polypeptides in prokaryotes Is most often carried out in Escherichia coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: (i) to increase expression of recombinant polypeptide; (ii) to increase the solubility of the recombinant polypeptide; and (iii) to aid in the purification of the recombinant polypeptide by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacla, Piscataway, N.J.) that fuse glutathione S-transferase (GST), maltose E binding polypeptide, or polypeptide A, respectively, to the target recombinant polypeptide.

Examples of suitable inducible non-fusion E. coli expression vectors include pTrc (Amrann et al., (1988) Gene 69:301-315) and pET 1 d (Studier et al., GENE EXPRESSION TECHNOLOGY: METHODS IN ENZYMOLOGY 185, Academic Press, San Diego, Calif. (1990) 60-89).

One strategy to maximize recombinant polypeptide expression in E. coli is to express the polypeptide in host bacteria with an impaired capacity to proteolytically cleave the recombinant polypeptide. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in the expression host, e.g., E. coil (see, e.g., Wada, et al, 1992. Nucl. Acids Res. 20: 2111-2118). Such alteration of nucleic acid sequences of the invention can be carried out by standard DNA synthesis techniques.

In another embodiment, the RAG expression vector is a yeast expression vector. Examples of vectors for expression in yeast Saccharomyces ceiivisae include pYepSeci (Baldari, et al., 1987. EMBO J. 6: 229-234), pMFa (Kurjan and Herskowitz, 1982. Cell 30: 933943), pJRY88 (Schultz et al., 1987. Gene 54: 113-123), pYES2 (Invitrogen Corporation, San Diego, Calif.), and picZ (InVitrogen Corp, San Diego, Calif.). Alternatively, RAG can be expressed in insect cells using baculovirus expression vectors. Baculovirus vectors available for expression of polypeptides in cultured insect cells (e.g., SF9 cells) include the pAc series (Smith, et al., 1983. Mol. Cell. Biol. 3: 2156-2165) and the PVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2P C (Kaufman, et al., 1987. EMBO J. 6:187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector Is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Vinoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byme and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the a-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule Is operatively-linked to a regulatory sequence in a manner that allows for expression (by transcription of the DNA molecule) of an RNA molecule that is antisense to a RAG mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends In Genetics, Vol. 1(1)1986.

Another aspect of the invention pertains to host cells into which a recombinant expression vector of the invention has been introduced. The terms “host cell” and “recombinant host cell” are used interchangeably herein. It Is understood that such terms refer not only to the particular subject cell but also to the progeny or potential progeny of such a cell. Because certain modifications may occur in succeeding generations due to either mutation or environmental influences, such progeny may not, in fact, be Identical to the parent cell, but are still included within the scope of the term as used herein.

A host cell can be any prokaryotic or eukaryotic cell. For example, RAG can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) Is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding RAG or can be introduced on a separate vector. Cells stably transfected with the introduced nucleic acid can be identified by drug selection (e.g., cells that have incorporated the selectable marker gene will survive, while the other cells die).

A host cell that includes a compound of the invention, such as a prokaryotic or eukaryotic host cell in culture, can be used to produce (i.e., express) recombinant RAG. In one embodiment, the method comprises culturing the host cell of invention (into which a recombinant expression vector encoding RAG has been introduced) in a suitable medium such that RAG is produced. In another embodiment, the method further comprises the step of isolating RAG from the medium or the host cell. Purification of recombinant polypeptides is well-known in the art and includes ion-exchange purification techniques, or affinity purification techniques, for example with an antibody to the compound. Methods of creating antibodies to the compounds of the present invention are discussed below.

E. RAG-Derived Chimeric and Fusion Polypeptides

The invention also provides for compounds that are RAG-derived chimeric or fusion polypeptides. As used herein, a RAG-derived “chimeric polypeptide” or “fusion polypeptide” comprises a RAG operatively-linked to a polypeptide having an amino acid sequence corresponding to a polypeptide that is not substantially homologous to the RAG, e.g., a polypeptide that is different from the RAG and that Is derived from the same or a different organism (i.e., non-RAG). Within a RAG-derived fusion polypeptide, the RAG can correspond to all or a portion of a RAG. In one embodiment, a RAG-derived fusion polypeptide comprises at least one biologically-active portion of a RAG, for example a fragment of RAG ID NO:1-281. In another embodiment, a RAG-derived fusion polypeptide comprises at least two biologically-active portions of a RAG. In yet another embodiment, a RAG-derived fusion polypeptide comprises at least three biologically-active portions of a RAG polypeptide. Within the fusion polypeptide, the term “operatively-linked” is intended to indicate that the RAG polypeptide and the non-RAG polypeptide are fused in-frame with one another. The non-RAG polypeptide can be fused to the N-terminus or C-terminus of the RAG.

In one embodiment, the fusion polypeptide is a GST-RAG fusion polypeptide in which the RAG sequences are fused to the N-or C-terminus of the GST (glutathione S-transferase) sequences. Such fusion polypeptides can facilitate the purification of recombinant RAG by affinity means.

In another embodiment, the fusion polypeptide Is a RAG polypeptide containing a heterologous signal sequence at its N-terminus. In certain host cells (e.g., mammalian host cells), expression and/or secretion of RAG can be increased through use of a heterologous signal sequence.

In yet another embodiment, the fusion polypeptide is a RAG-immunoglobulin fusion polypeptide in which the RAG sequences are fused to sequences derived from a member of the immunoglobulin superfamily. The RAG-immunoglobulin fusion polypeptides of the invention can be incorporated into pharmaceutical compositions and administered to a subject to inhibit an interaction between a RAG and a RAG receptor polypeptide on the surface of a cell, to thereby suppress RAG-mediated signal transduction in vivo. The RAG-immunoglobulin fusion polypeptides can be used to affect the bioavailability of a RAG, for example to target the compound to a particular cell or tissue having the requisite antigen. Inhibition of the RAG/RAG receptor interaction can be useful therapeutically for both the treatment of proliferative and differentiative disorders, as well as modulating (e.g., promoting or inhibiting) cell survival. Moreover, the RAG-immunoglobulin fusion polypeptides of the invention can be used as immunogens to produce anti-RAG antibodies in a subject, to purify RAG ligands, and in screening assays to identify molecules that inhibit the interaction of RAG with a RAG ligand.

### II. PREPARATION OF RAGs

A. Peptide synthesis of RAG Polypeptides

In one embodiment, a RAG polypeptide can be synthesized chemically using standard peptide synthesis techniques, e.g., solid-phase or solution-phase peptide synthesis. That Is, the compounds disclosed as RAG ID NOs:1-281 are chemically synthesized, for example, on a solid support or in solution using compositions and methods well known in the art, see, e.g., Fields, G. B. (I997) Solid-Phase Peptide Synthesis. Academic Press, San Diego.

The RAG polypeptide may be prepared by either Fmoc (base labile protecting group) or -Boc (acid labile a-amino protecting group) peptide synthesis. Following synthesis, RAGs then be rendered substantially free of chemical precursors or other chemicals by an appropriate purification scheme using standard polypeptide purification techniques for example, ion exchange chromatography, affinity chromatography, reverse-phase HPLC, e.g., using columns such as C-18, C-8, and C4, size exclusion chromatography, chromatography based on hydrophobic interactions, or other polypeptide purification method.

B. Production of RAG Polypeptides using Recombinant DNA Techniques

In another embodiment, RAG polypeptides are produced by recombinant DNA techniques, for example, overexpression of the compounds in bacteria, yeast, baculovirus or eukaryotic cells yields sufficient quantities of the compounds. Purification of the compounds from heterogeneous mixtures of materials, e.g., reaction mixtures or cellular lysates or other crude fractions, is accomplished by methods well known in the art, for example, ion exchange chromatography, affinity chromatography or other polypeptide purification methods. These can be facilitated by expressing the compounds described by RAG ID NOs:1-281 as fusions to a cleavable or otherwise inert epitope or sequence. The choice of an expression system and method of purification is well known to skilled artisans.

The polynucleotides provided by the present invention can be used to express recombinant compounds for analysis, characterization or therapeutic use; as markers for tissues in which the corresponding compound is preferentially expressed (either constitutively or at a particular stage of tissue differentiation or development or in disease states).

For recombinant expression of one or more the compounds of the invention, the nucleic acid containing all or a portion of the nucleotide sequence encoding the peptide may be inserted into an appropriate expression vector (i.e., a vector that contains the necessary elements for the transcription and translation of the inserted peptide coding sequence). In some embodiments, the regulatory elements are heterologous (ie., not the native gene promoter). Alternately, the necessary transcriptional and translational signals may also be supplied by the native promoter for the genes and/or their flanking regions.

A variety of host-vector systems may be utilized to express the peptide coding sequence(s). These include, but are not limited to: (i) mammalian cell systems that are infected with vaccinia virus, adenovirus, and the like; (ii) insect cell systems infected with baculovirus and the like; (iii) yeast containing yeast vectors or (iv) bacteria transformed with bacteriophage, DNA, plasmid DNA, or cosmid DNA. Depending upon the host-vector system utilized, any one of a number of suitable transcription and translation elements may be used.

Promoter/enhancer sequences within expression vectors may utilize plant, animal, insect, or fungus regulatory sequences, as provided in the invention. For example, promoter/enhancer elements from yeast and other fungi can be used (e.g., the GAL4 promoter, the alcohol dehydrogenase promoter, the phosphoglycerol kinase promoter, the alkaline phosphatase promoter). Alternatively, or in addition, they may include animal transcriptional control regions, e.g., (i) the insulin gene control region active within pancreatic cells (see, e.g., Hanahan, et al., 1985. Nature 315: 115-122); (ii) the immunoglobulin gene control region active within lymphoid cells (see, e.g., Grosschedl, et al., 1984. Cell 38: 647-658); (iii) the albumin gene control region active within liver (see, e.g., Pinckert, et al., 1987. Genes and Dev 1: 268-276; (iv) the myelin basic polypeptide gene control region active within brain oligodendrocyte cells (see, e.g., Readhead, et al., 1987. Cell 48: 703-712); and (v) the gonadotropin-releasing hormone gene control region active within the hypothalamus (see, e.g., Mason, et al., 1986. Science 234: 1372-1378), and the like.

Expression vectors or their derivatives include, e.g. human or animal viruses (e.g., vaccinia virus or adenovirus); insect viruses (e.g., baculovirus); yeast vectors; bacteriophage vectors (e.g., lambda phage); plasmid vectors and cosmid vectors.

A host cell strain may be selected that modulates the expression of inserted sequences of interest, or modifies or processes expressed peptides encoded by the sequences in the specific manner desired. In addition, expression from certain promoters may be enhanced in the presence of certain inducers in a selected host strain; thus facilitating control of the expression of a genetically-engineered compounds. Moreover, different host cells possess characteristic and specific mechanisms for the translational and post-translational processing and modification (e.g., glycosylation, phosphorylation, and the like) of expressed peptides. Appropriate cell lines or host systems may thus be chosen to ensure the desired modification and processing of the foreign peptide is achieved. For example, peptide expression within a bacterial system can be used to produce an unglycosylated core peptide; whereas expression within mammalian cells ensures “native” glycosylation of a heterologous peptide.

C. Preparation of RAG-Derived Chimeric or Fusion Polypeptide Compounds

A RAG-derived chimeric or fusion polypeptide compound of the invention can be produced by standard recombinant DNA techniques known in the art. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as approprdate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al., (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A RAG-encoding nucleic acid can be cloned into such an expression vector such that the fusion moiety is linked in-frame to the RAG encoding nucleic acid sequence.

D. Preparation of RAG Polypeptide Libraries

In addition, libraries of fragments of the nucleic acid sequences encoding RAGs can be used to generate a population of RAG fragments for screening and subsequent selection of variants of a RAG compound. In one embodiment, a library of coding sequence fragments can be generated by treating a double stranded PCR fragment of a nucleic acid sequence encoding RAG with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S1 nuclease, and ligating the resulting fragment library into an expression vector. By this method, expression libraries can be derived which encode N-terminal, C-terminal, and internal fragments of various sizes of the RAG polypeptides.

Various techniques are known in the art for screening gene products of combinatorial libraries made by point mutations or truncation, and for screening cDNA libraries for gene products having a selected property. Such techniques are adaptable for rapid screening of the DNA libraries generated by the combinatorial mutagenesis of RAG. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to Identify RAG variants. See, e.g., Arkin and Yourvan, 1992. Proc. Natl. Acad. Sci. USA 89: 7811-7815; Delgrave, et al, 1993. Polypeptide Engineering 6:327-331.

A library of RAG variant compounds can also be produced by, for example, enzymatically ligating a mixture of synthetic oligonucleotides into gene sequences such that a degenerate set of potential RAG sequences is expressible as individual polypeptides, or alternatively, as a set of larger fusion polypeptides (e.g., for phage display) containing the set of RAG sequences therein. There are a variety of methods that can be used to produce libraries of potential RAG variant compounds from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential RAG sequences. Methods for synthesizing degenerate oligonucleotides are well-known within the art. See, e.g., Narang, 1983. Tetrahedron 39: 3; Itakura, et a., 1984. Annu. Rev. Biochem. 53: 323; Itakura, et al., 1984. Science 198: 1056; Ike, et a., 1983. Nucl. Acids Res. 11:477.

E. Anti-RAG Antibodies

The invention provides compounds including polypeptides and polypeptide fragments suitable for use as immunogens to raise an-RAG antibodies. The compounds can be used to raise whole antibodies and antibody fragments, such as Fv, Fab or (Fab)₂, that bind immunospecifically to any of the RAGs of the invention, including bispecific or other multivalent antibodies.

An Isolated RAG polypeptide compound, or a portion or fragment thereof, can be used as an Immunogen to generate antibodies that bind to RAG or RAG polypeptides using standard techniques for polyclonal and monoclonal antibody preparation. The full-length RAG polypeptides can be used or, alternatively, the invention provides for the use of compounds including RAG fragments as immunogens. The RAG peptides comprises at least 4 amino acid residues of the amino acid sequence of RAG ID NOS:1-281, and encompasses an epitope of RAG such that an antibody raised against tie peptide fonts a specific immune complex with RAG polypeptide. Preferably, the antigenic peptide comprises at least 5, 8, 10, 15, 20, or 30 amino acid residues. Longer antigenic peptides are sometimes preferable over shorter antigenic peptides, depending on use and according to methods well known to those skilled in the art.

In certain embodiments of the invention, at least one epitope encompassed by the antigenic peptide is a region of RAG polypeptide that is located on the surface of the polypeptide (e.g., a hydrophilic region). As a means for targeting antibody production, hydropathy plots showing regions of hydrophilicity and hydrophobicity can be generated by any method well known in the art, including, for example, the Kyte Doolittle or the Hopp Woods methods, either with or without Fourier transformation (see, e.g., Hopp and Woods, 1981. Proc. Nat. Acad. Sci. USA 78: 3824-3828; Kyte and Doolittle, 1982. J. Mol. Biol. 157: 105-142, each incorporated herein by reference in their entirety).

As disclosed herein, RAG polypeptide or derivatives thereof, can be utilized as immunogens in the generation of antibodies that immunospecifically-bind these polypeptide components. In a specific embodiment, antibodies to human RAG polypeptides are disclosed. Various procedures known within the art can be used for the production of polyclonal or monoclonal antibodies to a RAG polypeptide sequence of RAG ID NO:1-281, or a derivative, fragment, analog or homolog thereof. Some of these polypeptides are discussed below.

For the production of polyclonal antibodies, various suitable host animals (e.g., rabbit, goat, mouse or other mammal) can be immunized by injection with the native polypeptide, or a synthetic variant thereof, or a derivative of the foregoing. An appropriate immunogenic preparation can contain, for example, recombinanty-expressed RAG or a chemically-synthesized RAG. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacteium parvum, or similar immunostimulatory compounds. If desired, the antibody molecules directed against RAG or RAG can be isolated from the mammal (e.g., from the blood) and further purified by well known techniques, such as polypeptide A chromatography to obtain the IgG fraction.

For preparation of monoclonal antibodies directed towards a particular RAG polypeptide, or derivatives, fragments, analogs or homologs thereof, any technique that provides for the production of antibody molecules by continuous cell line culture can be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al., 1985. in: MONOCLONAL ANTIBODIES AND CANCER THERAPY, Alan R. Liss, Inc., pp. 77-96). Human monoclonal antibodies can be utilized in the practice of the invention and can be produced by using human hybridomas (see, e.g., Cote, et al., 1983. Proc Natl Acad Sci USA 80: 2026-2030) or by transforming human B-cells with Epstein Barr Virus in vitro (see, e.g., Cole, et al., 1985. In: MONOCLONAL ANTIBODIES AND CANCER THERAPY, Alan R. Liss, Inc., pp. 77-96). Each of the above citations is incorporated herein by reference in their entirety. Synthetic dendromeric trees can be added a reactive amino acid side chains, e.g., lysine to enhance the immunogenic properties of RAG compounds. Also, CPG-dinucleotide technique can be used to enhance the immunogenic properties of RAG compounds.

According to the invention, techniques can be adapted for the production of single-chain antibodies specific to a RAG compound (see, e.g., U.S. Pat. No. 4,946,778). In addition, methods can be adapted for the construction of Fab expression libraries (see, e.g., Huse, et al., 1989. Science 246: 1275-1281) to allow rapid and effective Identification of monoclonal Fab fragments with the desired specificity for a RAG compound, e.g., a polypeptide or derivatives, fragments, analogs or homologs thereof. Non-human antibodies can be “humanized” by techniques wellknown in the art. See, e.g., U.S. Pat. No. 5,225,539. Antibody fragments that contain the idiotypes to a RAG compound can be produced by techniques known In the art including, but not limited to: (i) an F(ab′)₂ fragment produced by pepsin digestion of an antibody molecule; (ii) an Fab fragment generated by reducing the disulfide bridges of an F(ab′)₂ fragment; (iii) an Fab fragment generated by the treatment of the antibody molecule with papain and a reducing compound; and (iv) Fv fragments.

Additionally, recombinant anti-RAG antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No.184,187; European Patent Application No. 171,496; European Patent Application No. 173,494; PCT International Publication No. WO 86/01533; U.S. Patent Nos. 4,816,567; 5,225,539; European Patent Application No. 125,023; Better, et al., 1988. Science 240: 1041-1043; Liu, et al., 1987. Proc. Natl. Aced. Sci. USA 84: 34393443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314:446449; Shaw, et al., 1988. J. Natl. Cancer Inst 80: 1553-1559); Morrison (1985) Science 229:1202-1207; Oi, et al., (1986) BioTechniques 4:214; Jones, et al., 1986. Nature 321: 552-525; Verhoeyan, et al., 1988. Science 239: 1534; and Beidler, et al, 1988. J. Immunol. 141: 40534060. Each of the above citations is incorporated herein by reference in their entirety.

In one embodiment, methods for the screening of antibodies that possess the desired specificity to the RAG compounds include, but are not limited to, enzyme-linked Immunosorbent assay (ELISA) and other immunologically-mediated techniques known within the art. In a specific embodiment, selection of antibodies that are specific to a particular domain of a RAG polypeptide is facilitated by generation of hybridomas that bind to the fragment of a RAG polypeptide possessing such a domain. Thus, antibodies that are specific for a desired domain within a RAG, or derivatives, fragments, analogs or homologs thereof, are also provided herein.

Anti-RAG antibodies can be used in methods known within the art relating to the localization and/or quantitation of a RAG polypeptide or RAG compound (e.g., for use in measuring levels of the RAG polypeptide or RAG compound within appropriate physiological samples, for use in diagnostic methods, for use in imaging the polypeptide, and the like). In a given embodiment, antibodies for RAGs, or derivatives, fragments, analogs or homologs thereof, that contain the antibody derived binding domain, are utilized as pharmacologically-active compounds (hereinafter “Therapeutics”).

An anti-RAG antibody (e.g., monoclonal antibody) can be used to Isolate a RAG compound or RAG polypeptide by standard techniques, such as affinity chromatography or Immunoprecipitation. An and-RAG antibody can facilitate the purification of natural RAG polypeptide from cells and of recombinantiy-produced RAG expressed in host cells. Moreover, an anti-RAG antibody can be used to detect RAG polypeptide or RAG compounds (e.g., In a cellular lysate or cell supernatant) in order to evaluate the abundance and pattern of expression of the RAG polypeptide or RAG. Anti-RAG antibodies can be used diagnostically to monitor polypeptide levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidiniblotin and avidin/blotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ³²P, ¹²⁵I, ¹³¹I, ³⁵S, ³³P, ¹⁴C, ¹³C, or ³H.

### III. MEASURING THE BINDING OR BIOLOGICAL ACTIVITY OF RAGs

A. RAG Agonists and Antagonists

RAG compounds can function as either RAG receptor agonists (ie., mimetics) or as RAG receptor antagonists, as well as to the RAG, itself. An agonist of the RAG receptor (or RAG) can retain substantially the same, or a subset of, the biological activities of the naturally occurring form of the RAG polypeptide. An antagonist of the RAG receptor (or RAG) can inhibit one or more of the activities of the naturally occurring form of the RAG polypeptide by, for example, competitively binding to a downstream or upstream member of a cellular signaling cascade which includes the RAG receptor polypeptide. Thus, specific biological effects can be elicited by treatment with a variant of limited function. In one embodiment, treatment of a subject with a variant having a subset of the biological activities of the naturally occurring form of the polypeptide has fewer side effects in a subject relative to treatment with the naturally occurring form of the RAG polypeptide.

Accordingly, the compounds disclosed as RAG ID NOs:1-281 are used as agonists or antagonists of RAG polypeptides or RAG receptors, and are used, for example, to modulate signal transduction across a cell membrane of a cell expressing, e.g., RAG receptors. Modulation of signal transduction in such cells appears to occur as a result of specific binding interaction of RAG compounds with one or more cell surface receptors. Specific interaction means binding of the peptides to a RAG receptor with an equilibrium dissociation constant greater than 10⁶ M⁻¹. A cell surface bound membrane structure also may enhance the specificity of the binding interaction. Variants of the RAG polypeptides that function as either RAG agonists (i.e., mimetics) or as RAG antagonists can be identified by screening libraries of mutants (e.g., truncation mutants) of the RAG for RAG agonist or antagonist activity.

B. Measurement of RAG Binding

In one embodiment an RAG binding assay refers to a competitive assay format wherein a RAG receptor, its macromolecular ligand and an RAG are mixed under conditions suitable for binding between the RAG receptor and the ligand and assessing the amount of binding between the RAG receptor and its ligand. The amount of binding Is compared with a suitable control, which can be the amount of binding in the absence of the RAG, the amount of the binding in the presence of a known inhibitor, or both. The amount of binding can be assessed by any suitable method. Binding assay methods include, for example, ELISA, radioreceptor binding assays, scintillation proximity assays, cell surface receptor binding assays, fluorescence energy transfer assays, liquid chromatography, membrane filtration assays, and the like.

In a typical ligand/receptor binding assay useful in the practice of this invention, purified peptides having a known, quantifiable affinity for a pre-selected receptor (see, for example, Ten Dijke et al. (1994) Science 264:101-103, the disclosure of which is incorporated herein by reference) is labeled with a detectable moiety, for example, a radiolabel, a chromogenic label, or a fluorogenic label. Aliquots of purified receptor, receptor binding domain fragments, or cells expressing the receptor of interest on their surface are incubated with labeled peptide in the presence of various concentrations of the unlabeled peptide. The relative binding affinity of the peptide may be measured by quantitating the ability of the candidate (unlabeled peptide) to inhibit the binding of labeled peptide with the receptor. In performing the assay, fixed concentrations of the receptor and the peptide are incubated in the presence and absence of unlabeled peptide. Sensitivity may be increased by pre-incubating the receptor with the RAG analog before adding labeled peptide. After the labeled competitor has been added, suffident time is allowed for adequate competitor binding, and then free and bound labeled peptide are separated from one another, and one or the other measured. Labels useful in the practice of the screening procedures include radioactive labels (e.g., ¹²⁵I, ¹³¹I , ¹¹¹In or ⁷⁷Br), chromogenic labels, spectroscopic labels (such as those disclosed in Haughland (1994) “Handbook of Fluorescent and Research Chemicals 5 ed.” by Molecular Probes, Inc., Eugene, Oreg.), or conjugated enzymes having high turnover rates, for example, horseradish peroxidase, alkaline phosphatase, or beta-galactosidase, used in combination with chemiluminescent or fluorogenic substrates. Maximum binding signal is the signal measured in the presence of the native ligand, but without RAG present in the assay mixture. Background signal is the binding signal measured without the native ligand.

In a typical compound/receptor binding assay useful in the practice of this invention, purified reference compounds having a known, quantifiable affinity for a pre-selected receptor are labeled with a detectable moiety, for example, a radiolabel, a chromogenic label, or a fluorogenic label (see, for example, Ten Dijke et al. (1994) Science 264:101-103, the disclosure of which is incorporated herein by reference). Aliquots of purified receptor, receptor binding domain fragments, or cells expressing the receptor of interest on their surface are incubated with labeled compounds in the presence of various concentrations of the unlabeled compounds. The relative binding affinity of the peptide may be measured by quantitating the ability of the candidate (unlabeled peptide) to inhibit the binding of labeled peptide with the receptor. In performing the assay, fixed concentrations of the receptor and the peptide are incubated in the presence and absence of unlabeled peptide. Sensitivity may be increased by pre-incubating the receptor with the RAG compound before adding labeled peptide. After the labeled competitor has been added, sufficient time Is allowed for adequate competitor binding, and then free and bound labeled peptide are separated from one another, and one or the other measured. Labels useful in the practice of the screening procedures include radioactive labels (e.g., ¹²⁵I, ¹³¹I, ⁵¹Cr, ¹¹¹In or ⁷⁷Br), chromogenic labels, spectroscopic labels (such as those disclosed in Haughland (1994) “Handbook of Fluorescent and Research Chemicals 5 ed.” by Molecular Probes, Inc., Eugene, Oreg.), or conjugated enzymes having high turnover rates, for example, horseradish peroxidase, alkaline phosphatase, or beta-galactosidase, used in combination with chemiluminescent or fluorogenic substrates.

In another embodiment, a RAG binding assay refers to mixing a RAG binding ligand and an RAG compound under conditions suitable for binding between the RAG binding ligand and the RAG compound and assessing the degree of binding between the RAG binding ligand and the RAG compound, for example, measuring the dissociation constant and deriving the equilibrium binding constant through Scatchard or non-linear regression analysis. The amount of binding is compared with a suitable control the amount of the binding in the presence of a known inhibitor, or both. The amount of binding can be assessed by any suitable method. Binding assay methods include, for example, ELISA, radioreceptor binding assays, scintillation proximity assays, cell surface receptor binding assays, fluorescence energy transfer assays, liquid chromatography, membrane filtration assays, and the like.

Biophysical assays for the direct measurement of RAG binding to RAG-binding ligands are, for example, nuclear magnetic resonance, fluorescence, fluorescence polarization, surface plasmon resonance (BIACOR chips) and the like. RAG binding ligands, include, but are not limited to, RAG receptor, anti-RAG antibody, lipids, small molecules, and nucleic acids, e.g., DNA and RNA. Specific binding is determined by standard assays known in the art, for example, radioligand binding assays, ELISA, FRET, immunoprecipitation, SPR, NMR (2DNMR), mass spectroscopy and the like. Co-crystals of the RAG peptides and RAG binding ligands, for example, but not limited to, RAG receptor, anti-RAG antibody, lipids, small molecules, and nucleic acids, e.g., DNA and RNA, are also provided by the present invention as a method of determining molecular interactions. Conditions suitable for binding between the RAG ligand and an RAG compound will depend on the compound and its ligand and can be readily determined by one of ordinary skill in the art.

C. Measurement of RAG Biological Activity

The biological activity, namely the agonist or antagonist properties of RAG polypeptides or RAG compounds may be characterized using any conventional in vivo and in vitro assays that have been developed to measure the biological activity of the RAG compound or a RAG polypeptide. Specific in vivo assays for testing the efficacy of a RAG compound, polypeptide or analog, in an application to repair or regenerate damaged nervous tissue are disclosed in publicly available documents, which include, for example, U.S. Patent Application NOs. 2003/0215884; 2003/0118557; and 2003/0101945, each incorporated herein by reference in their entireties.

### IV. RAG TRANSGENIC ANIMALS

In still another embodiment, a transgenic animal, e.g., a mammal having a nucleic acid encoding a RAG polypeptide is provided. The host cells of the invention can also be used to produce non-human transgenic animals. For example, in one embodiment, a host cell of the invention Is a fertilized oocyte or an embryonic stem cell into which RAG polypeptide-coding sequences have been introduced. Such host cells can then be used to create non-human transgenic animals in which exogenous RAG sequences have been introduced into their genome or homologous recombinant animals in which endogenous RAG sequences have been altered. Such animals are useful for studying the function and/or activity of RAG polypeptide and for identifying and/or evaluating modulators of RAG polypeptide activity.

A transgenic animal of the invention can be created by intoducing a RAG-encoding nucleic acid into the male pronuclel of a fertilized oocyte (e.g., by microinjection, retroviral infection) and allowing the oocyte to develop in a pseudopregnant female foster animal. The RAG cDNA sequences of can be introduced as a transgene into the genome of a non-human animal. Intronic sequences and polyadenylation signals can also be included in the transgene to increase the efficiency of expression of the transgene. A tissue-specific regulatory sequence(s) can be operably-linked to the RAG transgene to direct expression of RAG polypepbide to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the RAG transgene in its genome and/or expression of RAG mRNA in tissues or cells of the animals. A transgenic founder animal can then be used to breed additional animals carrying the transgene. Moreover, transgenic animals carrying a transgene-encoding RAG polypeptide can further be bred to other transgenic animals carrying other transgenes.

In the homologous recombination vector, the RAG gene is flanked at its 5′- and 3′-termini by additional nucleic acid to allow for homologous recombination to occur between the exogenous RAG gene carried by the vector and an endogenous gene in an embryonic stem cell. The additional flanking nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is then introduced into an embryonic stem cell line (e.g., by electroporation) and cells in which the introduced RAG gene has homologously-recombined with an endogenous gene are selected. See, e.g., U, et al., 1992. Cell 69: 915.

To create a homologous recombinant animal, a vector is prepared which contains at least a portion of a RAG into which a deletion, addition or substitution has been introduced to thereby alter, e.g., functionally disrupt, the RAG. The RAG can be a human gene, but more preferably, Is a non-human homologue of a human RAG. For example, a mouse homologue of human RAG can be used to construct a homologous recombination vector suitable for altering an endogenous RAG in the mouse genome. In one embodiment, the vector is designed such that, upon homologous recombination, the endogenous RAG gene Is functionally disrupted (ie., no longer encodes a functional protein; also referred to as a knock out vector).

Alternatively, the vector can be designed such that, upon homologous recombination, the endogenous RAG Is mutated or otherwise altered but still encodes functional protein (e.g., the upstream regulatory region can be altered to thereby alter the expression of the endogenous RAG protein). In the homologous recombination vector, the altered portion of the RAG is flanked at its 5′ and 3′ ends by additional nucleic acid of the RAG to allow for homologous recombination to occur between the exogenous RAG carried by the vector and an endogenous RAG in an embryonic stem cell. The additional flanking RAG nucleic acid is of sufficient length for successful homologous recombination with the endogenous gene. Typically, several kilobases of flanking DNA (both at the 5′ and 3′ ends) are included in the vector. See e.g., Thomas et al., (1987) Cell 51:503 for a description of homologous recombination vectors. The vector is introduced into an embryonic stem cell line (e.g., by electroporation) and cells in which the introduced RAG has homologously recombined with the endogenous RAG are selected (see e.g., Li et al. (1992) Cell 69:915).

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823829; PCT International Publication Nos.: WO 90/11354; WO 91/01140; WO 92/0968; and WO 93/04169.

In another embodiment, transgenic non-human animals can be produced that contain selected systems that allow for regulated expression of the RAG transgene. One example of such a system is the cre/loxP recombinase system of bacteriophage P1. For a description of the cre/loxP recombinase system, See, e.g., Lakso, et al., 1992. Proc. Natl. Aced. Sci. USA 89: 6232-6236. Another example of a recombinase system is the FLP recombinase system of Saccharomyces cerevislae. See, O'Gorman, et al., 1991. Science 251:1351-2085. If a creAoxP recombinase system is used to regulate expression of the transgene, animals containing transgenes encoding both the Cre recombinase and a selected polypeptide are required. Such animals can be provided through the construction of “double” transgenic animals, e.g., by mating two transgenic animals, one containing a transgene encoding a selected polypeptide and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be Isolated and induced to exit the growth cycle and enter G0 phase. The quiescent cell can then be fused, e.g., through the use of electrical pulses, to an enucleated oocyte from an animal of the same species from which the quiescent cell Is isolated. The reconstructed oocyte Is then cultured such that It develops to formula or blastocyte and then transferred to pseudopregnant female foster animal. The offspring bome of this female foster animal will be a clone of the animal from which the cell (e.g., the somatic cell) is Isolated.

### VI. DISRUPTION OF RAG EXPRESSION USING SMALL INTERFERING RNA

Interfering RNA (RNAI) Is useful to introduce a double-stranded RNA (dsRNA) corresponding to a particular gene that will knock out the cell's own expression of that gene. This can be done in particular tissues at a chosen time. This often provides an advantage over conventional gene “knockouts” where the missing gene is carried in the germine and thus whose absence may kill the embryo before R can be studied. As such, in still another embodiment, the invention provides for the use of one or more intracellular small interfering RNA (siRNA) expression systems to regulate RAG expression. Expression systems for siRNA useful for the regulation of RAG expression in host cells as well as animal tissues are known in the art, e.g., U.S. Patent Application No. 20040002077, published Jan. 1, 2004, the contents of which is incorporated herein by reference in its entirety.

In one embodiment, the siRNA expression system comprises an antisense code DNA coding for the antisense RNA directed against a region of the RAG mRNA, a sense code DNA coding for the sense RNA directed against the same region of the RAG mRNA, and one or more promoters capable of expressing the antisense and sense RNAs from the antsense and sense code DNAS, respectively.

As used herein, “siRNA” means a small Interfering RNA that is a short-length double-stranded RNA that is not toxic in mammalian cells. The length is not limited to 21 to 23 bp long. There Is no particular limitation in the length of siRNA as long as it does not show toxicity. “siRNAs” can be, for example, 15 to 49 bp, preferably 15 to 35 bp, and more preferably 21 to 30 bp long. Alternatively, the double-stranded RNA portion of a final transcription product of sIRNA to be expressed can be, for example, 15 to 49 bp, preferably 15 to 35 bp, and more preferably 21 to 30 bp long. The double-stranded RNA portions of siRNAs in which two RNA strands pair up are not limited to the completely paired ones, and may contain nonpairing portions due to mismatch (the corresponding nudeotides are not complementary), bulge (lacking in the corresponding complementary nucleotide on one strand), and the like. Nonpairing portions can be contained to the extent that they do not interfere with siRNA formation. The “bulge” used herein preferably comprise 1 to 2 nonpairing nucleotides, and the double-stranded RNA region of siRNAs in which two RNA strands pair up contains preferably 1 to 7, more preferably 1 to 5 bulges. In addition, the “mismatch” used herein is contained in the double-stranded RNA region of siRNAs in which two RNA strands pair up, preferably 1 to 7, more preferably 1 to 5, in number. In a preferable mismatch, one of the nudeotides is guanine, and the other is uracil. Such a mismatch is due to a mutation from C to T, G to A, or mixtures thereof in DNA coding for sense RNA, but not particularly limited to them. Furthermore, in the present invention, the double-stranded RNA region of siRNAs in which two RNA strands pair up may contain both bulge and mismatched, which sum up to, preferably 1 to 7, more preferably 1 to 5 in number. Such nonpairing portions (mismatches or bulges, etc.) can suppress the below-described recombination between antisense and sense code DNAs and make the siRNA expression system as described below stable.

The terminal structure of RAG siRNA may be either blunt or cohesive (overhanging) as long as RAG siRNA enables to silence the RAG expression due to its RNAI effect. Furthermore, as long as RAG siRNA is able to maintain its gene silencing effect on the target gene, siRNA may contain a low molecular weight RNA (which may be a natural RNA molecule such as tRNA, rRNA or viral RNA, or an artificial RNA molecule), for example, in the overhanging portion at Its one end. In the case of a stem-loop RAG siRNA, either end (head or tail) of RNA with no loop structure may have a low molecular weight RNA. As described above, this low molecular weight RNA may be a natural RNA molecule such as tRNA, rRNA or viral RNA, or an artificial RNA molecule.

As used herein, “antisense RNA” Is an RNA strand having a sequence complementary to a RAG mRNA, and thought to induce RNAI by binding to the RAG mRNA. “Sense RNA” has a sequence complementary to the antisense RNA, and annealed to its complementary antisense RNA to form siRNA. These antisense and sense RNAs have been conventionally synthesized with an RNA synthesizer. These RNAs can be expressed intracellularly from DNAs coding for antisense and sense RNAs (antisense and sense code DNAs) respectively using the siRNA expression system.

To express antisense and sense RAG RNAs from the antsense and sense code DNAs respectively, the siRNA expression system can comprise one or more “promoter.” The type, number and location of the promoter can be arbitrarily selected as long as it is capable of expressing anbsense and sense code DNAs. As a simple construction of siRNA expression system, a tandem expression system can be formed, in which a promoter is located upstream of both antisense and sense code DNAs. This tandem expression system Is capable of producing siRNAs having the aforementioned cut off structure on both ends. In the stem-loop siRNA expression system (stem expression system), antisense and sense code DNAs are arranged in the opposite direction, and these DNAs are connected via a linker DNA to construct a unit. A promoter is linked to one side of this unit to construct a stem-loop siRNA expression system. As an example, DNA coding for the above-mentioned tRNA and such can be used as a linker DNA.

In both cases of tandem and stem-loop expression systems, the 5′ end may be have a sequence capable of promoting the transcription from the promoter. More specifically, in the case of tandem siRNA, the efficiency of siRNA production may be improved by adding a sequence capable of promoting the transcription from the promoters at the 5′ ends of antisense and sense code DNAs. In the case of stem-loop siRNA, such a sequence can be added at the 5′ end of the above-described unit. A transcript from such a sequence may be used in a state of being attached to siRNA as long as the target gene silencing by siRNA Is not hindered.

In either case of the above-mentioned tandem or stem expression system, either pol II or pol III promoter may be used as long as it is capable of producing the corresponding RAG RNAs from the above-described DNAs. Preferably, a pol III promoter suitable for expressing short RNAs such as SiRNAs can be used. Pol III promoters include the U6 promoter, tRNA promoter, retroviral LTR promoter, Adenovirus VA1 promoter, 5Sr RNA promoter, 7SK RNA promoter, 7SL RNA promoter, and H1 RNA promoter. The U6 promoter adds four uridine nucleotides to the 3′ end of RNA, thus the 3′ overhang of the finally produced siRNA can be freely made to be of 4, 3, 2, 1, or 0 nucleotide by providing the 5′ end sequence of the antisense and sense code DNAs with 0, 1, 2, 3 or 4 adenines. In the case of using other promoters, the number of 3′ overhanging nucleotide can be freely altered.

In the case of using pol III promoters, it is preferable to further provide a terminator at 3′ ends of sense and antisense code DNAs in order to express only the short RNAs and suitably terminate the transcription. Any terminator sequence can be used as long as it is capable of terminating the transcription initiated by the promoter. A sequence consisting of four or more consecutive adenine nucleotides, a sequence capable of forming the palindrome structure, etc. can be used.

Pol II promoters include the cytomegalovirus promoter, T7 promoter, T3 promoter, SP6 promoter, RSV promoter, EF-1α promoter, β-actin promoter, γ-globulin promoter, and SRα promoter. A pol II promoters produce not short RNAs as in the case of a pol III promoter but somewhat longer RNAs. Therefore, when pol II promoters are used, it is necessary to produce antisense or sense RNA by truncating somewhat longer RNA using a means to cleave RNA by self-processing such as a ribozyme.

If an inducible promoter is used as the promoter, RAG siRNA can be expressed at any desired timing. Such inducible promoters include the tetracycline-inducible U6 promoter (Ohkawa & Taira, Hum. Gene Ther. 11, 577-585 (2000). In addition, RAG siRNA expression may be Ussuespecifically induced using a tissue-specific promoter or a DNA recombination system such as Cre-loxP system. Moreover, instead of using a promoter inducible by drugs and such as described above, it is possible to control the siRNA production using, for example, a recombinase.

The RAG siRNA expression system comprising the abovementioned “promoter,” “antisense code DNA! and “sense code DNA” can be integrated as such into the chromosome to intracellularly express antisense and sense RAG RNAs, thereby producing RAG siRNA. Preferably, the RAG siRNA expression system is introduced into the target such as cells using a vector carrying the expression system to efficiently transfer the system. The vector usable in this invention can be selected depending on the target to be transfected, such as cells, and includes, for mammalian cells, viral vectors such as retrovirus vector, adenovirus vector, adeno-associated virus vector, vaccinia virus vector, intervirus vector, herpesvirus vector, alphavirus vector, EB virus vector, papilloma virus vector, and foamyvirus vector, and non-viral vectors including cationic liposome, ligand DNA complex, gene gun, etc. (Y. Niitsu, et al., Molecular Medicine 35: 1385-1395 (1998)), but not limited to them. Instead of viral vectors, dumbbell-shaped DNA (Zanta M. A. et al., Proc Natl Acad Sci USA. Jan 5, 1999; 96(1): 91-6), DNA modified so as to have nuclease resistance, or naked plasmids (Liu and Huang, J. Gene Med. 2001 November-December; 3(6): 569-76) are also useful in regulating RAG expression The antisense and sense RAG RNAs may be expressed in the same vector or in different vectors. For example, the construction for expressing both antisense and sense RAG RNAs from the same vector can be prepared by linking a promoter, such as a pol III promoter capable of expressing short RNA, upstream of antisense and sense code DNAs to form antisense and sense RNA expression cassettes, and inserting these cassettes into a vector either in the same direction or opposite directions. It is also possible to construct an expression system in which antisense and sense code DNAs are arranged on different strands in the opposite orientation so as to pair up. This construction may comprise one double-stranded DNA comprising antisense and sense RNA coding strands (DNA coding for siRNA), and promoters on both sides facing to each other so as to express the antisense and sense RNAs from the respective DNA strands. In this case, to avoid the addition of excess sequences downstream of the sense and antisense RNAs, it is preferable to place a terminator at 3′ ends of the respective strands (strands coding for antisense and sense RNAs). The terminator may be a sequence of four or more consecutive adenine (A) nucleotides. Alternatively, a construct capable of expressing the above-described stem-loop RAG siRNAs, it is also possible to form a unit in which both antisense and sense code DNAs are arranged in the opposite orientation on the same DNA strand via a linker, and link the resulting unit downstream of a single promoter.

### VII. PHARMACEUTICAL COMPOSITIONS

The RAG-encoding nucleic acid molecules, RAG polypeptides, RAG compounds and anti-RAG antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, polypeptide, or antibody and a pharmaceutically acceptable carrier. As used herein, “pharmaceutically acceptable carrier” is intended to include any and all solvents, dispersion media, coatings, antibacterial and antifungal compounds, isotonic and absorption delaying compounds, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringers solutions, dextrose solution, and 5% human serum albumin. Liposomes and non-aqueous vehicles such as fixed oils may also be used. The use of such media and compounds for pharmaceutically active substances is well known in the art. Except insofar as any conventional media or compound Is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor Elm (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antfungal compounds, for example, parabens, chlorobutanol, phenol, ascorbic acid, thimerosal, and the like. In many cases, it will be preferable to include isotonic compounds, for example, sugars, polyalcohols such as manitol, sorbitol, or sodium chloride, or a combination thereof in the composition. Prolonged absorption of the injectable compositions can be brought about by including in the composition a compound which delays absorption, for example, aluminum monostearate and gelatin.

Sterile injectable solutions can be prepared by incorporating the active compound (e.g., a RAG polypeptide or anti-RAG antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared as pharmaceutical compositions in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) cr by stereotactic injection (see, e.g., Chen, et al., 1994. Proc. Natl. Aced. Sci. USA 91: 3054-3057). The pharmaceutical preparation of the gene therapy vector can include the gene therapy vector in an acceptable diluent, or can comprise a slow release matrix in which the gene delivery vehicle is imbedded. Altematively, where the complete gene delivery vector can be produced intact from recombinant cells, e.g., retroviral vectors, the pharmaceutical preparation can include one or more cells that produce the gene delivery system. The pharmaceutical compositions can be included in a container, pack, or dispenser together with instructions for administration.

### VIII. SCREENING AND DETECTION METHODS

the compounds of the invention can be used to express RAG polypeptides (e.g., via a recombinant expression vector in a host cell in gene therapy applications), to detect RAG mRNA (e.g., in a biological sample) or a genetic lesion in a RAG gene, and to modulate RAG activity, as described further, below. In addition, the RAG polypeptides can be used to screen drugs or compounds that modulate the RAG polypeptide or RAG activity or expression as well as to treat disorders characterized by insufficient or excessive production of RAG polypeptides or production of RAG polypeptide forms that have decreased or aberrant activity compared to RAG wild-type polypeptide. In addition, the anti-RAG antibodies of the invention can be used to detect and isolate RAG or RAG polypeptides and modulate their activity. Accordingly, the present invention further includes novel compounds identified by the screening assays described herein and uses thereof for treatments as described, supra.

### IX. SCREENING ASSAYS

The Invention provides for methods for identifying modulators, ie., candidate or test compounds or compounds (e.g., peptides, peptidomimetics, small molecules or other drugs) that bind to RAG receptor or RAG polypeptides or have a stimulatory or inhibitory effect on, e.g., RAG receptor or RAG polypeptide expression or activity (also referred to herein as “screening assays”). The invention also includes compounds identified in the screening assays described herein.

In one embodiment, the invention includes assays for screening candidate or test compounds which bind to or modulate the activity of RAG receptor or RAG polypeptides or biologically-active portions thereof. The compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

Libraries of chemical and/or biological mixtures, such as fungal, bacterial, or algal extracts, are known in the art and can be screened with any of the assays described as well as those known to skilled artisans. Examples of methods for the synthesis of molecular libraries can be found in the scientific literature, for example in: DeWitt, et al., 1993. Proc. Natl. Acad. Sci. U.S.A. 90: 6909; Erb, et al., 1994. Proc. Natl. Acad. Sc. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds can be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404406; Cwirla, et al., 1990. Proc. Natl. Acad. Sci. U.S.A. 87: 6378-6382; Felid, 1991. J. Mol. Biol. 222: 301-310; Ladner, U.S. Pat. No. 5,233,409.).

Determining the ability of a compound to modulate the activity of a RAG polypeptide can be accomplished, for example, by determining the ability of the RAG polypeptide to bind to or interact with a RAG target molecule. A target molecule is a molecule that a RAG polypeptide binds or interacts with, for example, a molecule on the surface of a cell which expresses a RAG interacting polypeptide, a molecule on the surface of a second cell, a molecule in the extracellular milieu, a molecule associated with the internal surface of a cell membrane or a cytoplasmic molecule. A RAG target molecule can be a non-RAG molecule or a RAG polypeptide or polypeptide of the invention. In one embodiment, a RAG target molecule is a component of a signal transduction pathway that facilitates transduction of an extracellular signal (e.g., a signal generated by binding of a compound to a membrane-bound RAG receptor molecule) through the cell membrane and into the cell. The target, for example, can be a second intracellular polypeptide that has catalytic activity or a polypeptide that facilitates the association of downstream signaling molecules with RAG receptor polypeptide. The compounds of the present invention either agonize or antagonize such interactions and the resultant biological responses, measured by the assays described.

Determining the ability of the RAG polypeptide to bind to or interact with a RAG target molecule can be accomplished by one of the methods described above for determining direct binding. In one embodiment, determining the ability of the RAG polypeptide to bind to or interact with a RAG target molecule can be accomplished by determining the activity of the target molecule. For example, the activity of the target molecule can be determined by detecting induction of a cellular second messenger of the target (i.e., intracellular Ca²⁺, diacylglycerol, IP3, etc.), detecting catalytic/enzymatic activity of the target and appropriate substrate, detecting the induction of a reporter gene (comprising a RAG-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a RAG polypeptide or biologically-active portion thereof with a test compound and determining the ability of the test compound to bind to the RAG polypeptide or biologically-active portion thereof. Binding of the test compound to the RAG polypeptide can be determined either directly or indirectly as described above in one such embodiment, the assay comprises contacting the RAG polypeptide or biologically-active portion thereof with a known compound which binds RAG to form an assay mixture, contacting the assay mixture with a test compound, and determining the ability of the test compound to interact with a RAG polypeptide, wherein determining the ability of the test compound to interact with a RAG polypeptide comprises determining the ability of the test compound to preferentially bind to RAG or biologically-active portion thereof as compared to the known compound.

In still another embodiment, an assay is a cellfree assay comprising contacting RAG polypeptide or biologically-active portion thereof with a test compound and determining the ability of the test compound to modulate (e.g. stimulate or inhibit) the activity of the RAG polypeptide or biologically-active portion thereof. Determining the ability of the test compound to modulate the activity of RAG can be accomplished, for example, by determining the ability of the RAG polypeptide to bind to a RAG target molecule by one of the methods described above for determining direct binding. In an alternative embodiment, determining the ability of the test compound to modulate the activity of RAG polypeptide can be accomplished by determining the ability of the RAG polypeptide to further modulate a RAG target molecule. For example, the catalytic/enzymatic activity of the target molecule on an appropriate substrate can be determined as described, supra.

In yet another embodiment, the cell-free assay comprises contacting the RAG polypeptide or biologically-active portion thereof with a known compound which binds RAG polypeptide to form an assay mixture, contacting the assay mixture with a test compound, and determining the ability of the test compound to interact with a RAG polypeptide, wherein determining the ability of the test compound to interact with a RAG polypeptide comprises determining the ability of the RAG polypeptide to preferentially bind to or modulate the activity of a RAG target molecule.

In more than one embodiment of the above assay methods of the invention, it can be desirable to immobilize either RAG polypeptide or Its target molecule to facilitate separation of complexed from uncomplexed forms of one or both of the polypeptides, as well as to accommodate automation of the assay. Binding of a test compound to RAG polypeptide, or interaction of RAG polypeptide with a target molecule in the presence and absence of a candidate compound, can be accomplished in any vessel suitable for containing the reactants. Examples of such vessels include microtiter plates, test tubes, and micro-centrifuge tubes. In one embodiment, a fusion polypeptide can be provided that adds a domain that allows one or both of the polypeptides to be bound to a matrix. For example, GST-RAG fusion polypeptides or GST-target fusion polypeptides can be adsorbed onto glutathione sepharose beads (Sigma Chemical,.St. Louis, Mo.) or glutathione derivatized microtiter plates, that are then combined with the test compound or the test compound and either the non-adsorbed target polypeptide or RAG polypeptide, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Altematively, the complexes can be dissociated from the matrix, and the level of RAG polypeptide binding or activity determined using standard techniques.

Other techniques for Immobilizing polypeptides on matrices can also be used in the screening assays of the invention. For example, either the RAG polypeptide or its target molecule can be immobilized utilizing conjugation of biotin and streptavidin. Biotinylated RAG polypeptide or target molecules can be prepared from biotin-NHS (N-hydroxy-succinimide) using techniques well-known within the art (e.g., biotinylation kit, Pierce Chemicals, Rockford, Ill.), and Immobilized in the wells of streptavidin-coated 96 well plates (Pierce Chemical). Alternatively, antibodies reactive with RAG polypeptide or target molecules, but which do not interfere with binding of the RAG polypeptide to Its target molecule, can be derivatized to the wells of the plate, and unbound target or RAG polypeptide trapped in the wells by antibody conjugation. Methods for detecting such complexes, in addition to those described above for the GST-immobilized complexes, include immunodetection of complexes using antibodies reactive with the RAG polypeptide or target molecule, as well as enzyme-linked assays that rely on detecting an enzymatic activity associated with the RAG polypeptide or target molecule.

In another embodiment, modulators of RAG polypeptide expression are identified in a method wherein a cell is contacted with a candidate compound and the expression of RAG mRNA or polypeptide in the cell is determined. The level of expression of RAG mRNA or polypeptide in the presence of the candidate compound Is compared to the level of expression of RAG mRNA or polypeptide in the absence of the candidate compound. The candidate compound can then be Identified as a modulator of RAG mRNA or polypeptide expression based upon this comparison. For example, when expression of RAG mRNA or polypeptide is greater (ie., statistically significantly greater) in the presence of the candidate compound than in its absence, the candidate compound Is identified as a stimulator of RAG mRNA or polypeptide expression. Alternatively, when expression of RAG mRNA or polypeptide is less (statistically significantly less) in the presence of the candidate compound than in Its absence, the candidate compound is identified as an inhibitor of RAG mRNA or polypeptide expression. The level of RAG mRNA or polypeptide expression in the cells can be determined by methods described herein for detecting RAG mRNA or polypeptide.

In yet another aspect of the invention, the RAG polypeptides can be used as “bait polypeptides” in a two-hybrid assay or three hybrid assay (see, e.g., U.S. Pat. No. 5,283,317; Zervos, etal., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other polypeptides that bind to or interact with RAG (“RAG-binding polypeptides” or “RAG-bp”) and modulate RAG activity. Such RAG-binding polypeptides are also likely to be involved in the propagation of signals by the RAG polypeptides as, for example, upstream or downstream elements of the RAG pathway.

The two-hybrid system Is based on the modular nature of most transcription factors, which consist of separable DNA-binding and activation domains. Briefly, the assay utilizes two different DNA constructs. In one construct, the gene that codes for RAG is fused to a gene encoding the DNA binding domain of a known transcription factor (e.g., GAL-4). In the other construct, a DNA sequence, from a library of DNA sequences that encodes an unidentified polypeptide (“prey” or “sample”) is fused to a gene that codes for the activation domain of the known transcription factor. If the “bait” and the “prey” polypeptides are able to interact, in vivo, forming a RAG-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be Isolated and used to obtain the cloned gene that encodes the polypeptide which interacts with RAG.

In still another embodiment, a system comprising structural information relating to the RAG atomic coordinates can be obtained by biophysical techniques, e.g., x-ray diffraction. Binding between a RAG peptide and a compound can be assessed by x-ray diffraction to determine the x-ray crystal structure of the RAG complexes, e.g., target polypeptide/drug complex. Alternatively; NMR may be used to analyze the change in chemical shifts observed after a compound binds with the RAG polypeptide. Such approaches may be used to screen for compounds based on their binding interaction with RAG polypeptide.

The invention further pertains to RAG compounds identified by the aforementioned screening assays and uses thereof for treatments as described herein.

### X. DETECTION ASSAYS

A. Detection of RAG Expression

An exemplary method for detecting the presence or absence of RAG in a biological sample involves obtaining a biological sample from a test subject and contacting the biological sample with a compound or a compound capable of detecting RAG polypeptide or nucleic add (e.g., mRNA, genomic DNA) that encodes RAG polypeptide such that the presence of RAG is detected in the biological sample. A compound for detecting RAG mRNA or genomic DNA is a labeled nucleic acid probe capable of hybridizing to RAG mRNA or genomic DNA. The nucleic acid probe can be, for example, a full-length RAG nucleic acid or a portion thereof, such as an oligonucleotide of at least 5, 15, 30, 50, 100, 250 or 500 nucleotides in length and sufficient to specifically hybridize under stringent conditions to RAG mRNA or genomic DNA. Other suitable probes for use in the diagnostic assays of the invention are described herein.

An example of a compound for detecting a RAG polypeptide is an antibody raised against RAG ID NOS:1-281, capable of binding to the RAG polypeptide, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (ie., physically linking) a detectable substance to the probe or antibody, as well as indirect labeling of the probe or antibody by reactivity with another compound that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect RAG mRNA, polypeptide, or genomic DNA in a biological sample in vitro as well as in vivo. For example, in vitro techniques for detection of RAG mRNA include Northern hybridizations and in situ hybridizations. In vitro techniques for detection of RAG polypeptide include enzyme linked immunosorbent assays (ELISAs), Western blots, immunoprecipitations, and immunofluorescence. In vitro techniques for detection of RAG genomic DNA include Southern hybridizations. Furthermore, in vivo techniques for detection of RAG polypeptide include introducing into a subject a labeled anti-RAG antibody. For example, the antibody can be labeled with a radioactive marker whose presence and location in a subject can be detected by standard Imaging techniques. In one embodiment, the biological sample contains polypeptide molecules from the test subject. Altematively, the biological sample can contain mRNA molecules from the test subject or genomic DNA molecules from the test subject. A preferred biological sample is a peripheral blood leukocyte sample Isolated by conventional means from a subject.

In another embodiment, the methods further involve obtaining a control biological sample from a control subject, contacting the control sample with a compound or compound capable of detecting RAG polypeptide, mRNA, or genomic DNA, such that the presence of RAG polypeptide, mRNA or genomic DNA is detected in the biological sample, and comparing the presence of RAG polypeptide, mRNA or genomic DNA in the control sample with the presence of RAG polypeptide, mRNA or genomic DNA in the test sample.

The invention also encompasses kits for detecting the presence of RAG in a biological sample. For example, the kit can comprise: a labeled compound or compound capable of detecting RAG polypeptide or mRNA in a biological sample; means for determining the amount of RAG in the sample; and means for comparing the amount of RAG in the sample with a standard. The compound or compound can be packaged in a suitable container. The kit can further comprise instructions for using the kit to detect RAG polypeptide or nucleic acid.

B. Predictive Medicine

The invention also pertains to the field of predictive medicine in which diagnostic assays, prognostic assays, pharmacogenomics, and monitoring clinical trials are used for prognostic (predictive) purposes to treat prophylactically a subject. Accordingly, one aspect of the invention relates to diagnostic assays for determining RAG target molecule expression as well as RAG target molecule activity, in the context of a biological sample (e.g., blood, serum, cells, tissue) to thereby determine whether an individual Is afflicted with a disease or disorder, or Is at risk of developing a disorder, associated with aberrant RAG target molecule expression or activity.

The invention also provides for prognostic (or predictive) assays for determining whether an individual Is at risk of developing a disorder associated with RAG target molecule expression or activity. Such assays can be used for prognostic or predictive purpose to thereby prophylactically treat an individual prior to the onset of a disorder characterized by or associated with a RAG target polypeptide. Furthermore, the methods of the present invention can also be used to assess whether an individual expresses a RAG target molecule or a polymorphic form of the target polypeptide in instances where a RAG of the present invention has greater affinity for the RAG target molecule for its polymorphic form (or vice versa).

The levels of certain polypeptides in a particular tissue (or in the blood) of a subject may be indicative of the toxicity, efficacy, rate of clearance or rate of metabolism of a given drug when administered to the subject. The methods described herein can also be used to determine the levels of such polypeptide(s) in subjects to aid in predicting the response of such subjects to these drugs. Another aspect of the invention provides methods for determining RAG polypeptide activity in an individual to thereby select appropriate therapeutic or prophylactic compounds for that individual (referred to herein as “pharmacogenomics”). Pharmacogenomics allows for the selection of compounds (e.g., drugs) for therapeutic or prophylactic treatment of an individual based on the genotype of the individual (e.g., the genotype of the individual examined to determine the ability of the individual to respond to a particular compound.)

C. Prognostic Assays

The binding of a RAG compound to a RAG compound target molecule, e.g., RAG receptor, can be utilized to identify a subject having or at risk of developing a disorder associated with RAG compound target molecule expression or activity (which are described above). Alternatively, the prognostic assays can be utilized to identify a subject having or at risk for developing the disease or disorder. Thus, the invention provides a method for identifying a disease or disorder associated with aberrant RAG compound target expression or activity in which a test sample is obtained from a subject and RAG compound binding or activity Is detected, wherein the presence of an alteration of RAG compound binding or activity is diagnostic for a subject having or at risk of developing a disease or disorder associated with aberrant RAG compound target expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered a compound (e.g., an agonist, antagonist, peptidomimetic, polypeptide, peptide, nucleic acid, small molecule, or other drug candidate) to treat a RAG-associated disease or disorder associated with aberrant RAG compound target expression or activity. For example, such methods can be used to determine whether a subject can be effectively treated with a compound for a RAG-associated disorder. Thus, the invention provides methods for determining whether a subject can be effectively treated with a compound for a disorder associated with aberrant RAG compound target expression or activity in which a test sample is obtained and RAG compound target Is detected using RAG compound (e.g., wherein the presence of RAG compound target molecule is diagnostic for a subject that can be administered the compound to treat a disorder associated with aberrant RAG compound target molecule expression or activity).

The level of the RAG compound target molecule in a blood or tissue sample obtained from a subject is determined and compared with the level found in a blood sample or a sample from the same tissue type obtained from an individual who Is free of the disease. An overabundance (or under abundance) of the RAG compound target molecule in the sample obtained from the subject suspected of having the RAG-associated disease compared with the sample obtained from the healthy subject is indicative of the RAG-associated disease in the subject being tested. Further testing may be required to make a positive diagnosis.

There are a number of diseases in which the degree of overexpression (or underexpression) of certain RAG compound target molecules, referred to herein as “prognostic polypeptides', is known to be indicative of whether a subject with the disease is likely to respond to a particular type of therapy or treatment. Thus, the method of detecting a RAG compound target molecule in a sample can be used as a method of prognosis, e.g., to evaluate the likelihood that the subject will respond to the therapy or treatment. The level of the relevant prognostic polypeptide in a suitable tissue or blood sample from the subject is determined and compared with a suitable control, e.g., the level in subjects with the same disease but who have responded favorably to the treatment. The degree to which the prognostic polypeptide is overexpressed (or underexpressed) in the sample compared with the control may be predictive of likelihood that the subject will not respond favorably to the treatment or therapy. The greater the overexpression (or underexpression) relative to the control, the less likely the subject will respond to the treatment. There are a number of diseases in which the degree of overexpression (or underexpression) of certain target polypeptides, referred to herein as “predictive polypeptides”, Is known to be indicative of whether a subject will develop a disease.

Thus, the method of detecting a RAG compound target molecule in a sample can be used as a method of predicting whether a subject will develop a disease. The level of the relevant predictive polypeptide in a suitable tissue or blood sample from a subject at risk of developing the disease is determined and compared with a suitable control, e.g., the level in subjects who are not at risk of developing the disease. The degree to which the predictive polypeptide is overexpressed (or underexpressed) in the sample compared with the control may be predictive of likelihood that the subject will develop the disease. The greater the overexpression (or underexpression) relative to the control, the more likely the subject will development the disease.

The methods described herein can be performed, for example, by utilizing pre-packaged diagnostic kits comprising at least one probe reagent, e.g., RAG compound described herein, which can be conveniently used, e.g., in clinical settings to diagnose patients exhibiting symptoms or family history of a disease or Illness involving a RAG compound target molecule. Furthermore, any cell type or tissue in which RAG compound target is expressed can be utilized in the prognostic assays described herein.

### XI. METHODS OF TREATMENT

The invention provides for both prophylactic and therapeutic methods of treating a subject at risk of (or susceptible to) a disorder or having a disorder associated with aberrant RAG polypeptide or RAG target molecule expression or activity. RAG and RAG target molecules, such as RAG receptors, play a role in cell differentiation. Cell differentiation Is the central characteristic of tissue morphogenesis. Tissue morphogenesis is a process involved in adult tissue repair and regeneration mechanisms. The degree of morphogenesis in adult tissue varies among different tissues and is related, among other things, to the degree of cell turnover in a given tissue.

Accordingly, the compounds of the present invention can be used to treat both acute and chronic neuronal damage, e.g., brain and spinal cord trauma, stroke, cerebral palsy, multiple sclerosis, Parkinson's disease, Alzheimer's disease, ALS, stroke and peripheral neuropathies. The compounds of the present invention are also useful to promote nerve growth following organ transplantation, e.g., heart transplantation surgery. For example, RAG-based therapeutic compositions are useful to induce regenerative healing of nervous tissue defects such as lesions, as well as, to preserve or restore healthy metabolic properties in diseased tissue, e.g., nerve tissue.

RAG compounds, can be used in the prophylaxis or treatment of traumatic brain injury, e.g., stroke, head injury see, e.g., Miyake et al., Brain Res. 2002 May 10; 935(1-2): 24-31. Miyake and coworkers observed alterations in factors involved in the regeneration of the neuronal network in the hippocampus of rats with microsphere embolism (ME). RAG compounds of the present invention are, therefore, useful to promote regeneration of functional neurons in the ischemic penumbra following sustained cerebral ischemia.

Head trauma triggers both excitotoxic and apoptotic neurodegeneration in the developing rat brain-(Bittilgau et al., Neurotox Res. 2003; 5(7): 475-90P). Excitotoxic neurodegeneration develops and subsides rapidly (within hours) whereas apoptotic cell death occurs in a delayed fashion over several days following the initial traumatic insult. Apoptotic neurodegeneration contributes in an age-dependent fashion to neuronal injury following head trauma, with the immature brain being exceedingly sensitive. Molecular and biochemical studies indicate that both extrinsic and intrinsic mechanisms are involved in pathogenesis of apoptotic cell death following trauma. RAG compounds of the present invention are, therefore, useful to promote regeneration of functional neurons in a subject after acute trauma, e.g., head injury.

RAG compounds, can be used in the prophylaxis or treatment of spinal cord trauma (see, e.g., Seizer, Lancet Neurol. 2003 Mar; 2(3): 157-66). Molecules that are found in the extracellular environment at a CNS lesion site, or that are associated with myelin, inhibit axon growth. In addition, neuronal changes—such as an age-dependent reduction in concentrations of cyclic AMP - render the neuron less able to respond to axotomy by a rapid, forward, actin-dependent movement. An alternative mechanism, based on the protrusive forces generated by microtubule elongation or the anterograde transport of cytoskeletal elements, may underlie a slower form of axon elongation that happens during regeneration in the mature CNS. Therapeutic approaches that restore the extracellular CNS environment or the neuron's characteristics back to a more embryonic state increase axon regeneration and improve functional recovery after injury. These advances in the understanding of regeneration in the CNS have major implications for neurorehabilitation and for the use of axonal regeneration as a therapeutic approach to disorders of the CNS such as spinal-cord injury. RAG compounds of the present Invention are, therefore, useful to promote regeneration of functional neurons in the injured spinal cord. RAG compounds, can be used in the prophylaxis or treatment of cerebral palsy (see, e.g., Vanek et al., Eur J Neurosci. 1998 Jan; 10(1): 45-56). Myelin contains potent inhibitors of neurite growth which have been implicated in the failure of long-distance regeneration of nerve fibers within the CNS. These myelin-associated neurite growth inhibitors may also be involved in the stabilization of neural connections by suppressing sprouting and fiber growth. After lesions of the CNS in neonatal animals, extensive rearrangements of the remaining fiber systems have been observed. In the rat, this plasticity of neuronal connections is severely restricted following the first few weeks of postnatal life, coincident with the progression of myelination of the nervous system. A well-studied example of postnatal plasticity is the sprouting of one corticospinal tract (CST) into the denervated half of the spinal cord after unilateral motor cortex or pyramidal lesions. In the hamster and rat, significant CST sprouting is restricted to the first 10 postnatal days. Here we show that very extensive sprouting of corticospinal fibers occurs after deafferentations as late as P21 if myelination is prevented by neonatal X-irradiation in the rat lumbar spinal cord. Sprouted fibers from the intact CST cross the midline and develop large terminal arbors in the denervated spinal cord, suggesting the establishment of synaptic connections. Our results suggest that myelin and its associated neurite growth inhibitors play an important role in the termination of neurite growth permissive periods during postnatal CNS development Corticospinal sprouting subsequent to lesions early in life, i.e. in the absence of myelin-associated neurite growth inhibitors may explain the frequent occurrence of mirror movements in patients with hemiplegic cerebral palsy. RAG compounds of the present invention are, therefore, useful to promote regeneration of functional neurons in a subject with cerebral palsy.

RAG compounds, can be used in the prophylaxis or treatment of multiple sclerosis (see, e.g., Pantano et al., Neurolmage. 2002 Dec; 17(4): 1837-43). The contribution of corUcospinal tract damage to cortical motor reorganization after a single clinical attack of multiple sclerosis has been studied by Pantano and coworkers (Pantano et al, Neuroimage. 2002 Dec; 17(4): 1837-43). A significant correlation between the time since clinical onset of multiple sclerosis and activation in motor areas suggests that cortical reorganization develops gradually in concomitance with the subclinical accumulation of tissue damage. RAG compounds of the present invention are, therefore, useful to promote regeneration of functional neurons in a subject with multiple sclerosis.

RAG compounds, can be used in the prophylaxis or treatment of neuronal diseases such as Parkinson's disease, ALS and Alzheimer's disease (see, e.g., Varon et al., Dev Neurosci. 1983-84; 6(2): 73-100). The clinical potential of neuronotrophic and neurite-promoting factors such as RAGs Is recognized in the art. Specifically, neuronal maintenance and neuritic growth during development are increasingly recognized as being under the extrinsic control of neuronotrophic- and neurite-promoting agents. Protein agents (‘factors’) are the most studied but not the only molecules exerting such controls. It appears increasingly likely that adult neurons in situ are equally subject to similar extrinsic regulations. Two recently studied in vivo models for peripheral and central neural regeneration have demonstrated trauma-related accumulations of neuronotrophic- and neurite-promoting factors in the adult rat, in close temporal correlation with neuronal maintenance and axonal regrowth, respectively. Deficits in the supply or utilization of similar factors may underlie neuronal or glial regressive processes in aging, and in selected neuronal diseases such as Parkinson's disease, ALS and Alzheimer's disease. RAG compounds of the present invention are, therefore, useful to promote regeneration of functional neurons a subject with Parkinson's disease, Alzheimer's disease or ALS.

RAG compounds, can be used in the prophylaxis or treatment of Parkinson's disease (see, e.g., Luquin et al., Neurologia. 1991 Oct; 6(8): 287-94). MPTP administration, preferably to mice and primates, induces a selective damage of substantia nigra dopaminergic cells. Intrinsic mechanisms mediating MPTP induced toxicity, remain still under evaluation. Parkinson's disease is caused by the progressive degeneration of the neurons of the substantia nigra. RAG compounds of the present invention are, therefore, useful to promote regeneration of functional neurons in a subject with Parkinson's disease.

RAG compounds, can be used in the prophylaxis or treatment of Alzhelmer's disease (see, e.g., Mori and Morli, J Neurosci Res. 2002 Nov. 1; 70(3): 264-73). Mori and Morii studied SCG10-related neuronal growth-associated proteins in neural development, plasticity, degeneration, and aging. Neuronal growth-associated proteins (nGAPs) are in general neuron-specific gene products whose expression correlates tightly with neuronal process outgrowth and/or regeneration, and are mostly good downstream targets of neurotrophin stimulation. Expression of genes encoding nGAPs such as GAP43, SCG10, and stathmin Is upregulated following lesioning of cortical and hippocampal regions of the adult rat brain. In the brains of aged animals, however, the magnitude of the response is reduced, whereas the time course of the response Is mostly unchanged when compared with that for brains of young ones. Expression of GAP-43 and stathmin is reduced by aging, and is also changed in age-related neurodegenerative conditions such as Alzheimer's disease in humans. Certain nGAPs are induced during long-term potentiation (LTP) and also during critical periods of song-learning and ocular dominance column formation in birds and cats, respectively. Recent evidence further supports the idea that functional synaptic modulation is often associated with remodeling of synaptic structures. These results suggest that neurotrophin-responsive nGAPs serve as molecular markers of neuronal plasticity during development and aging, and that the neuronal plasticity decreases, at least in certain neuronal circuits, in the aged brain and neurodegenerative diseases. RAG compounds of the present invention are, therefore, useful to promote regeneration of functional neurons in a subject with Alzheimer's disease.

RAG compounds, can be used in the prophylaxis or treatment of ALS (see, e.g., Parhad et al., Ann Neurol. 1992 Jun.; 31(6): 593-7). RAG expression, e.g., GAP-43 gene expression, is increased in anterior horn cells of subjects suffering from amyotrophic lateral sclerosis. In amyotrophic lateral sclerosis (ALS), neuronal loss and axonal degeneration occur in motor neurons. Although there is limited axonal regeneration, surviving motor neurons send collateral sprouts to denervated muscle fibers. GAP-43, a protein enriched in growth cones and synaptic terminals, is thought to have a role in axonal elongation and synaptogenesis. RAG compounds of the present invention are, therefore, useful to promote regeneration of functional neurons in a subject with ALS.

RAG compounds, can be used in the prophylaxis or treatment of peripheral neuropathy (e.g., diabetic neuropathy), see, e.g., (Yasuda et al., Prog Neurobiol. 2003 Mar; 69(4): 229-85). Diabetic neuropathy is the most common peripheral neuropathy in western countries. Although every effort has been made to clarify the pathogenic mechanism of diabetic neuropathy, thereby devising its ideal therapeutic drugs, neither convinced hypotheses nor unequivocally effective drugs have been established. In view of the pathologic basis for the treatment of diabetic neuropathy, it is Important to enhance nerve regeneration as well as prevent nerve degeneration. Nerve regeneration or sprouting in diabetes may occur not only in the nerve trunk but also in the dermis and around dorsal root ganglion neurons, thereby being implicated in the generation of pain sensation. Thus, inadequate nerve regeneration unequivocally contributes to the pathophysiologic mechanism of diabetic neuropathy. In this context, the research on nerve regeneration in diabetes should be more accelerated. Indeed, nerve regenerative capacity has been shown to be decreased in diabetic patients as well as in diabetic animals. Disturbed nerve regeneration in diabetes has been ascribed at least in part to all or some of decreased levels of neurotrophic factors, decreased expression of their receptors, altered cellular signal pathways and/or abnormal expression of cell adhesion molecules, although the mechanisms of their changes remain almost unclear. In addition to their steady-state changes in diabetes, nerve injury induces injury-specific changes in individual neurotrophic factors, their receptors and their intracellular signal pathways, which are dosely linked with altered neuronal function, varying from neuronal survival and neurite extension/nerve regeneration to apoptosis. RAG compounds of the present invention are, therefore, useful to promote regeneration of functional neurons in a subject with peripheral neuropathy, e.g., diabetic neuropathy.

RAG compounds, can be used in to promote reinnervation of transplanted tissue, e.g., transplanted heart tissue, see, e.g., (Bengel et al., N Engl J Med. 2001 Sep. 6; 345(10): 731-8). Studies on the effect of sympathetic reinnervation on cardiac performance after heart transplantation support the functional importance of reinnervabon in transplanted hearts. RAG compounds of the present invention are, therefore, useful to promote reinnervation of transplanted tissue, e.g., transplanted heart tissue.

### XII. DISEASE AND DISORDERS

Diseases and disorders that are characterized by increased (relative to a subject not suffering from the disease or disorder) levels or biological activity of RAG polypeptides or RAG target molecules can be treated with RAG-based therapeutic compounds that antagonize (ie., reduce or inhibit) activity, which can be administered in a therapeutic or prophylactic manner. Therapeutic compounds that can be utilized include, but are not limited to: (i) an aforementioned RAG peptide, or analogs, derivatives, fragments or homologs thereof; (ii) antPRAG antibodies to an aforementioned peptide; (iii) nucleic acids encoding RAG peptide; (iv) administration of antisense nucleic acid and nucleic acids that are “dysfunctional” (i.e., due to a heterologous insertion within the coding sequences of coding sequences to a RAG peptide) that are utilized to “knockout” endogenous function of RAG peptide by homologous recombination (see, e.g., Capecchi, 1989. Science 244:1288-1292) or (v) ‘knockdown’ endogenous function of RAG peptide by siRNA expression technology; or (vi) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity of RAG or RAG target molecule can be treated with RAG-based therapeutic compounds that increase (Le., are agonists to) RAG activity or RAG polypeptide activity. Therapeutics that upregulate activity can be administered in a therapeutic or prophylactic manner. Therapeutics that can be utilized include, but are not limited to, RAG polypeptide, or analogs, derivatives, fragments or homologs thereof; or an agonist that increases bioavailability.

Increased or decreased levels can be readily detected by quantifying RAG-induced peptides and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are wellknown within the art include, but are not limited to, immunoassays (e.g., by Western blot analysis, immunoprecipitation followed by sodium dodecyl sulfate (SDS) polyacrylamide gel electrophoresis, Immunocytochemistry, etc.) and/or hybridization assays to detect expression of mRNAs (e.g., Northern assays, dot blots, In situ hybridization, and the like).

A. Prophylactic Methods

In one aspect, the invention provides a method for preventing, in a subject, a disease or condition associated with an aberrant RAG polypeptide or RAG target molecule expression or activity, by administering to the subject a RAG or RAG mimetic that modulates RAG polypeptide or RAG target molecule expression or at least one RAG polypeptde or RAG target molecule activity.

Subjects at risk for a disease that is caused or contributed to by aberrant RAG polypeptide or RAG target molecule expression or activity can be identified by, for example, any or a combination of diagnostic or prognostic assays as described herein. Administration of a prophylactic compound can occur prior to the manifestation of symptoms characteristic of the aberrancy, such that a disease or disorder is prevented or, alternatively, delayed in its progression. Depending upon the type of aberrancy, for example, a RAG, RAG mimetic, or anti-RAG antibody, which acts as an RAG agonist or RAG antagonist compound can be used for treating the subject. The appropriate compound can be determined based on screening assays described herein.

B. Therapeutic Methods

Another aspect of the invention includes methods of modulating RAG polypeptides or RAG target molecule expression or activity in a subject for therapeutic purposes. The modulatory method of the invention involves contacting a cell with a compound of the present invention, that modulates one or more of the activities of the RAG polypeptide or RAG target molecule activity associated with the cell. A compound that modulates a RAG polypeptide or RAG target molecule activity is described herein, such as a nucleic acid or a polypeptide, a naturally-occurring cognate ligand of a RAG polypeptide, a RAG peptide, an anti-RAG antibody, a RAG mimetic, or a small molecule. In one embodiment, the compound stimulates one or more RAG polypeptide or RAG target molecule activity. Examples of such stimulatory compounds include a RAG polypeptide and a nucleic acid molecule encoding RAG that has been introduced into the cell. In another embodiment, the compound inhibits one or more RAG polypeptide or RAG target molecule activity, e.g., anti-RAG antibodies. These modulatory methods can be performed in vitro (e.g., by culturing the cell with the compound) or, altematively, in vivo (e.g., by administering the compound to a subject). As such, the invention provides methods of treating an individual afflicted with a RAG-associated disease or disorder characterized by aberrant expression or activity of a RAG polypeptide or RAG target molecule or nucleic acid molecules encoding them. In one embodiment, the method involves administering a compound (e.g., a compound identified by a screening assay described herein), or combination of compounds that modulates (e.g., up-regulates or down-regulates) RAG polypeptide or RAG target molecule expression or activity. In another embodiment, the method involves administering a RAG polypeptide or nucleic acid molecule encoding RAG as therapy to compensate for reduced or aberrant RAG polypeptide or RAG target molecule expression or activity.

Stimulation of RAG polypeptide or RAG target molecule activity Is desirable in situations in which RAG polypeptide or RAG target molecule Is abnormally downregulated and/or in which increased RAG activity is likely to have a beneficial effect. One example of a situation when a RAG antagonist is useful is where a subject has a disorder characterized by aberrant cell proliferation and/or differentiation (e.g., aberrant nerve growth factor production, brain cancer, neuroblastoma).

C. Determination of the Biological Effect of the RAG-based therapeutic

In various embodiments of the invention, suitable in vitro or in vivo assays are performed to determine the effect of a specific RAG-based therapeutic and whether its administration is indicated for treatment of the affected tissue in a subject.

In various specific embodiments, in vitro assays can be performed with representative cells of the type(s) involved in the patient's disorder, to determine If a given RAG-based therapeutic exerts the desired effect upon the cell type(s). Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

D. Prophylactic and Therapeutic Uses of the Compositions of the Invention

The RAG compounds of the present invention are useful in potential prophylactic and therapeutic applications Implicated in a variety of disorders in a subject including, but not limited to: those involving development, differentiation, and activation of nerve cells; in diseases or pathologies of cells in brain trauma, cancer, neurological diseases, neurodegenerative disorders, Alzheimer's disease, ALS, and Parkinson's disease. The compounds of the present invention are also useful to promote nerve growth following organ transplantation, e.g., heart transplantation surgery. See also, supra, Methods of Treatment.

As an example, a cDNA encoding the RAG polypeptide compound can be useful in gene therapy, and the polypeptide can be useful when administered to a subject in need thereof. By way of non-limiting example, the compositions of the invention will have efficacy for treatment of patients suffering from the above mentioned disorders. Both the novel nucleic acid encoding the RAG polypeptide or fragments thereof, are also be useful in diagnostic applications.

### EXAMPLES

The following examples are intended to be non-limiting illustrations of certain embodiments of the present invention. All references cited are hereby incorporated herein by reference in their entireties.

### Example 1

### Studies Demonstrating that Fibroblast Growth Factor-Inducible-14 is induced in Axotomized Neurons and Promotes Neurite Growth

### I. GENERAL

Validation and functional studies were conducted for several selected genes derived from the initial screen. Four genes were studied further myosin-X, SOX11, FLRT3, and Fn14. No prior studies investigated their role in the nervous system generally or in axonal regeneration specifically.

Fn14 is a transmembrane protein (Meighan-Mantha et al., 1999) reported to be a receptor for tumor necrosis-like weak inducer of apoptosis (TWEAK), a member of the tumor necrosis factor (TNF) superfamily of structurally related cytokines (Wiley et al., 2001). It has been reported that TWEAK Is an angiogenic factor and that TWEAK binding to Fn14 promotes the proliferation of both human endothelial cells, leading to angiogenesis (Lynch et al., 1999; Wiley et al., 2001; Harada et al., 2002; Donohue et al., 2003), and astrocytes (Desplat-Jego et al., 2002). There has been was no prior reported neuronal function for Fn14. We showed that Fn14 is induced in dorsal root ganglia (DRG) neurons during sciatic nerve regeneration and promotes neurite outgrowth through a mechanism that appears to involve the Rho family GTPase, Rac1.

### II. METHODS AND PROCEDURES

A. Animal Surgery and RNA Isolation

Adult male C57BLU6 mice were anesthetized by intraperitoneal injection of 2,2,2-tribromoethanol (Sigma-Aldrich, St. Louis, Mo.). The sciatic nerve at the mid-thigh level was exposed and transected. For retrograde labeling, fluoro-gold (FG) was applied to the cut proximal end of the nerve at the time of the transection. Animals were killed 1, 4, 7, 14, 28, or 56 d after injury, and the ipsilateral DRG neurons from L4, L5, and L6 were placed into liquid nitrogen. Contralateral DRG neurons served as control. Total RNA was extracted with an RNeasy mini kit (Qiagen, Valencia, Calif.).

B. Microarray Analysis

The total RNA from control and axotomized DRG samples was reverse-transcribed with T7-(dT)24 primer (Affymetrix, Santa Clara, Calif.), and double-stranded CDNA was synthesized with Superscript Choice system (Invitrogen, Carlsbad, Calif.). Biotin-labeled cRNA was generated with a BioArray High Yield RNA transcript labeling kit (Enzo Diagnostics, Inc, NY). These cRNA samples were hybridized with Mouse Genome U74 A, B, and C arrays from Affymetrix.

C. Northern Blot Analysis

As templates, myosin-X (bp 4-627, BF536234 [GenBank]), SOX11 (bp 40-382, BE854207 [GenBank]), FLRT3 (bp 18-472, BE286611 [GenBank]), and TWEAK (bp 78-561, AW763237 [GenBank]) were amplified using PCR reaction from I.M.A.G.E. clones (Incyte Genomics, St. Louis, Mo.). The Fn14 cDNA fragment (bp 13-41 1, NM013749) was amplified from a pcDNAI/Neo plasmid encoding influenza hemagglutinin (HA)-tagged Fn14 (Meighan-Mantha et al., 1999). Probes were synthesized from these cDNAs by random priming in the presence of [-32P]dCTP. Northern blot analysis was conducted as described with 5 μg samples of total RNA from DRG tissue or PC12 cells (Bonilla et al., 2002).

D. In situ Hybridization

In situ hybridization was performed as described previously (Tanabe et at., 1999, 2000). Fn14 cDNA (bp 22-411, NM03749) and FLRT3 cDNA (bp 18-472, BE286611 [GenBank]) were amplified by PCR and subcloned into pCR2Topo (Invitrogen). The SOX11 cDNA clone (bp 1873-2896, AF009414 [GenBank]) was a generous gift from P. Koopman (University of Queensland, Queensland, Australia). In vitro transcription from linearized plasmids was performed using T7 and SP6 RNA polymerase and [-35S]UTP (DuPont NEN, Wilmington, Del.) to prepare both antisense and sense riboprobes. After hybridization to tissue sections, mRNA localization was assessed with emulsion autoradiography.

E. Herpes Simplex Virus Preparation

The enhanced green fluorescent protein (GFP) cDNA was amplified from pEGFP-N3 (Clontech, Palo Alto, Calif.) and ligated to the pHSVprPUC vector (Nakamura et al., 1998; Takahashi et al., 1998, 1999). The full coding region of Fn14, Fn14 without the ectocellular domain (aa 77-129), or Fn14 without the cytoplasmic domain (aa 1-103) was amplified by PCR and ligated to the N-terminus EGFP coding region of pHSVprPUC-GFP to express Fn14-GFP fusion protein. The ectocellular-deleted Fn14 mutant contains the translational start and signal sequence from pSecTag2/Hygro vector (Invitrogen). These plasmids were transfected into 2-2 cells with Lipofectamine (Invitrogen) and superinfected with 5dl1.2 herpes simplex helper virus 1 d later as described (Nakamura et al., 1998; Takahashi et al., 1998, 1999).

F. Cell Culture, Transfection, and Herpes Simplex Virus Infection Assay

PC12 cells were grown in DMEM with 10% horse serum and 5% fetal bovine serum. For herpes simplex virus (HSV) infection, cells were plated on glass chamber slides coated with poly-L-lysine (100 μg/mi) plus laminin (10 mg/ml) and then incubated in serum-free DMEM with N2 supplement. Cells were fixed 24 hr after infection and stained with rhodamine-phalloidin for visualization of F-actin. GAP-43 was visualized with anti-GAP43 polyclonal antibody and cyanine 3 (Cy3)-conjugated antiabbit IgG antibody. For cotransfection of individual Rho family proteins and Fn14, Bos expression vectors including myc-tagged Cdc42 (G12V, wild, or T17N), Rac1 (G12V, wild, or T17N) and RhoA (G14V, wild, or T19N) were kindly provided by Y. Takal (Osaka University, Osaka, Japan). PC12 cells were transfected with the plasmids expressing Rho family proteins using Lipofectamine2000 (Invitrogen) and replated to poly-L-lysine 100 μg/ml) plus laminin (10 mg/ml)-coated chamber slides 24 hr after transfection. They were infected with HSVFn14-GFP virus and fixed 24 hr after infection. Recombinant human TWEAK was obtained from Peprotech (Rocky Hill, N.J.), and the extracellular domain of Fn14 fused with the Fc portion of murine IgG (Fn14-Fc) was prepared as described (Donohue et al., 2003).

DRG neurons were removed from the L46 level from 8- to 10-week-old mice with or without sciatic nerve crush injuries 1 week previously. Neurons were dissociated, cultured, and infected with HSV preparations as described previously (Bonilla et al., 2002). After 24 hr in culture, cells were fixed, and neurite outgrowth per GFP-expressing viral-infected neurons was scored as described (Bonilla et al., 2002). In these cultures, 50-80% of neurons were infected. Some cultures were collected after 24 hr, and protein was analyzed by immunoblot for GFP and Fn14 expression.

COS-7 cells were transfected with Bos myc-Cdc42 (G12V, wild, or T17N), Rac1 (G12V, wild, or T17N), or RhoA (G14V, wild, or T19N) and pEGFP-N3 containing coding region of Fn14 (Meighan-Mantha et al., 1999) using Fugene 6 (Roche, Indianapolis, Ind.). The cells were fixed 24 hr after transfecton, and Rho family GTPases were visualized with 9E10 c-Myc-antibody (Sigma-Aldrich) and Cy3-conjugated anti-mouse IgG antibody (Sigma-Aldrich).

G. Immunoprecipitation

Human embryonic kidney (HEK) 293T cells were transfected with pcDNA1-Fn14-HA (Meighan-Mantha et al., 1999) and Bos vectors encoding myc-Rac1 or myc-Cdc42. Transfected 293T cells were harvested and lysed at 4° C. for 1 hr in 50 mM Tris-HCl, pH 8, 150 mM NaCl, 5 mM MgCl2, 1% Triton X-00, 0.5% deoxycholic acid, 0.1% SDS, and complete proteinase inhibitor mixture (Roche). The lysates were centrifuged at 13,000×g at 4° C. for 20 min, and the supernatants were collected. Samples were then incubated with an anti-Myc-conjugated agarose (Santa Cruz Biotechnology, Santa Cruz, Calif.) for 14 hr at 4° C. The agarose beads were centrifuged and washed five times with wash buffer, 50 mM Tris-HCl, pH 8,150 mM NaCl, 5 mM MgCl2, 1% Triton X-100, and complete proteinase inhibitor mixture. The bound Fn14 was detected by immunoblotting with anti-HA monoclonal antibody (Roche). DRG samples were immunoblotted using anti-GFP (Clontech) and anti-Fn14 antibodies (Meighan-Mantha et al., 1999).

H. Rac1 Activity Assay

HEK293T cells were transfected with pcDNAI/Neo-Fn14-HA or pcDNAI/Neo and lysed with 50 mM Tris-HCl, pH 7.5, 500 mM NaCl, 0.1% SDS, 0.5% deoxycholate, 1% Triton X-100, 10 mM MgCl2, and complete proteinase inhibitor. The lysate was centrifuged at 13,000×g at 4° C. for 20 min, and the supematant was incubated with 10 μg of GST-p21-binding domain of PAK-1 coupled to glutathione agarose at 4° C. for 45 min. The agarose beads were collected by centrifugation and washed with 50 mM Tris-HCl, pH 7.5, 150 mM NaCl, 1% Triton X-100, 10 mM MgCl2, and complete proteinase inhibitor. The bound GTP-Rac1 was detected by immunoblotting with anti-Rac1 monoclonal antibody (Upstate Biotechnology, Lake Placid, N.Y.).

### III. RESULTS

A. Myosin-X, SOX11, and FLRT3 Expression are Linked to Regeneration-Development of Injured Neuronal Cells

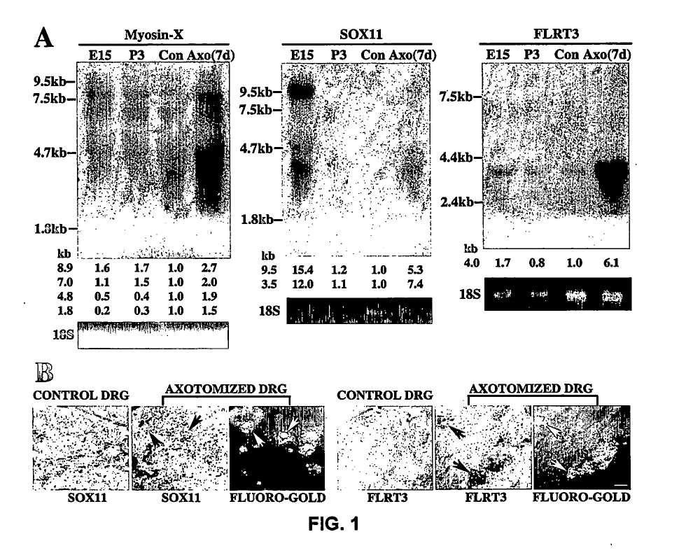

Studies were conducted to examine the expression profile of myosin-X, SOX11, and FLRT3 before and after nerve injury. As a first step, the microarray expression changes were validated by Northern blot analysis of control and 7 d post-sciatic nerve injury DRG RNA samples (FIG. 1A). Myosin-X has two transcript forms in adult mouse tissue, a full-length form (8.7 kb) and a shorter form deleting N-terminal head region (7.0 kb), with a third short isoform (4.8 kb) in embryonic tissue (Berg et al., 2000; Yonezawa et al., 2000). In myosin-X Northern blot hybridizations, four weak signals (8.7, 7.0, 4.8, and 1.8 kb) were observed seen in control DRG tissue, and all of these transcripts were upregulated to some degree by nerve injury. In embryonic day 15 (E15) and neonatal day 3 (P3) samples, the 8.7 and 7.0 kb transcripts were more highly expressed than in uninjured adult DRG.

For SOX11, a previous study had demonstrated a single transcript of 3.5 kb in the whole mouse embryo (Hargrave et al., 1997), whereas in human and chicken two transcripts (9.5 and 3.0 kb) were observed (Jay et al., 1995; Uwanogho et al., 1995). In the present study, a low level of SOX11 mRNA expression was detected (3.5 kb) in control adult DRG. In contrast, axotomized DRG samples expressed much higher levels of the 3.5 kb SOX11 transcript, with detectable levels of three minor transcripts (9.5, 4.8, and 2.0 kb). Uninjured E15 DRG expression of all four SOX1 1 transcripts was strong, and expression was downregulated to adult levels by P3.

In human tissues, FLRT3 was detected as a single transcript of 4.4 kb (Lacy et al., 1999). In the present studies, the mouse control adult ORG Northern blots, a single weak signal was also present at 4.0 kb. FLRT3 mRNA expression was dramatically increased after nerve injury, and E15 DRG had higher expression than adult control DRG.

In brief, the microarray data for myosin-X, SOX11, and FLRT3 were confirmed by Northern blotting. In each case, the up-regulation after injury mirrored the high expression during development, thus linking these genes and their polypeptides to regeneration-development of injured neuronal cells. Although the Northern analysis demonstrated DRG e, pression, it did not distinguish which cells express the mRNA of interest.

In situ hybridization studies were conducted for SOX11 and FLRT3 (FIG.1 B) to confirm neuronal expression of these genes and further support a direct role for SOX1 1 and FLRT3 genes in the process of axonal regeneration. Injured neurons were identified by retrograde labeling with FG applied at the time of injury to the proximal cut end of the sciatic nerve. Increased silver grain density reflecting hybridization to either FLRT3 or SOX11 mRNA was observed over cells in the injured DRG sections. Moreover, the increased grain density corresponded to the fluorescent label, demonstrating that these genes were induced in axotomized neurons. The induction was not DRG neuron-subtype specific as most of the FG-positive cells were SOX11 or FLRT3 positive after injury.

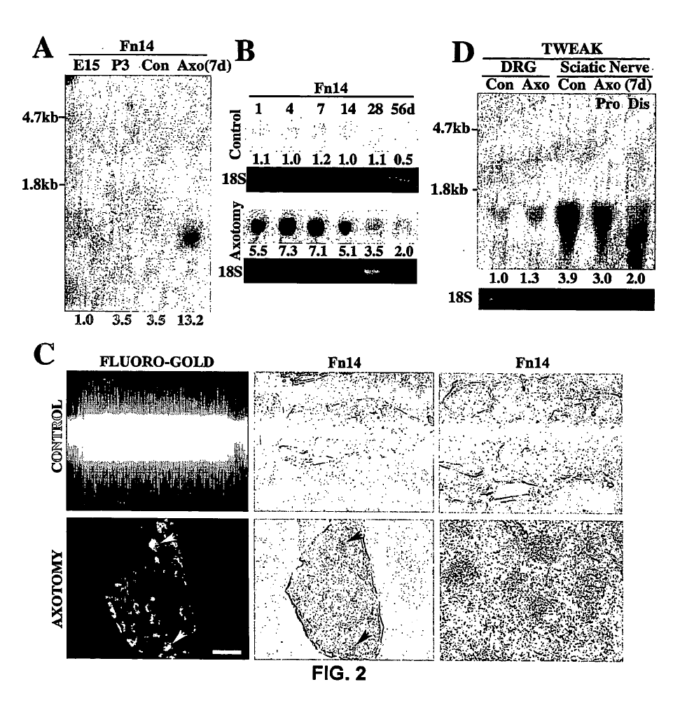

B. Fn14 and TWEAK are Linked to Regeneraton-Development of injured Neuronal Cells Studies were conducted to examine the expression profile of Fn14 and TWEAK before and after nerve injury. That is, validation studies by Northern analysis and in situ hybridization were also performed for the Fn14 gene. The expression level of the 1.2 kb Fn14 mRNA was barely detectable in uninjured adult DRG orin developing DRG (EI5 and P3); however, Fn14 mRNAwas induced after axotomy (FIG. 2A). Thus, Fn14 is a RAG not subject to developmental regulation but specific to the regeneration process. Fn14 expression was increased 1 d after axotomy and was elevated further by postoperative days 4-7. Thereafter, Fn14 mRNA expression gradually decreased to control levels by 56 d after axotomy (FIG. 2B). By in situ hybridization Fn14 expression was localized to injured DRG neurons retrogradely labeled with fluoro-gold from the axotomy site. There was little specificity of Fn14 regulation among different DRG populations (FIG. 2C) as >80% of fluoro-gold-stained cells exhibit increased Fn14 mRNA.

Recently, Fn14 was identified as a receptor for the TNF-related cytokine TWEAK (VViley et al., 2001). Therefore, TWEAK mRNA expression was examined in the DRG and in the proximal and distal nerve stumps before and after sciatic nerve injury (FIG. 2D). TWEAK mRNA level was the same in the control and axotomized DRG. TWEAK was moderately expressed in control sciatic nerve, and expression was downregulated by nerve injury. Coordinated regulation of Fn14 and TWEAK gene expression during sciatic nerve regeneration was not observed.

The fourth RAG examined, Fn14, was induced in neurons during peripheral nerve regeneration but is not present during neural development. In this regard, it does not fit the developmental paradigm for axonal regeneration. There is growing precedence for such a separation of these processes. Our previous studies on SPRRIA identified another RAG that distinguished regenerative axon growth from developmental axonal extension (Bonilla et al., 2002). Similarly, JAK-STAT signaling appears to play a role selectively in regenerative growth from DRG (Liu and Snider, 2001). Although TWEAK can serve as a ligand for Fn14 in endothelial cells (Wiley et al., 2001; Harada etal., 2002; Donohue et al., 2003), we did not find evidence for compensatory or parallel regulation of Fn14 and TWEAK during nerve regeneration. Similarly, functional studies of axon outgrowth and Fn14 argue against a role for TWEAIK.

Thus, the function of Fn14 in this context Is ligand independent. Ligand-independent, TNF receptor-associated factor (TRAF)-dependent Fn14 activation of nuclear factor-B (NF-B) has been reported (Brown et al., 2003). Thus, the absence of a role for TWEAK is perhaps not surprising for these Fn14-sensitive neuronal cultures.

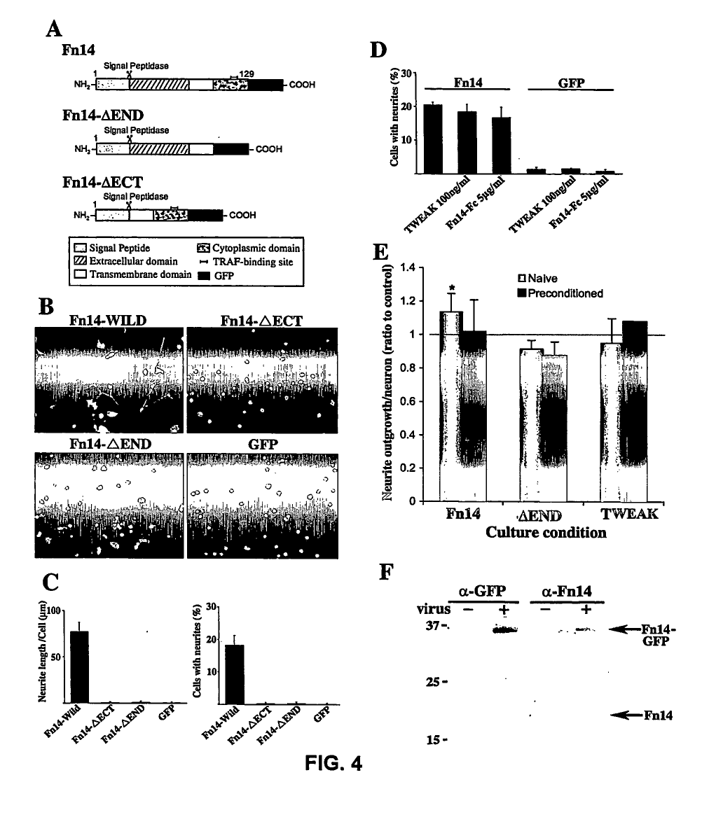

Fn14 overexpression promotes growth-cone formation and neurite outgrowth Although PC12 cells normally possess low levels of Fn14, overexpression of the protein induces filopodia, lamelipodia, and neuritogenesis. In NGF-dtfferentiated cultures, Fn14 overexpression promotes growth cone formation. A direct role for Fn14 in forming filopodia, neurites, and growth cones is supported by the localization of Fn14 protein to these structures and its colocalizatlon with F-actin and GAP-43. These effects are clear in PC12 cells that otherwise exhibit few or no neurites and growth cones. For DRG neurons, axon growth Is robust in culture, and minimal enhancement by Fn14 overexpression is observed in comparison only with cells expressing a potentially dominant-negative Fn14 truncation mutant (FIG. 4).

The lack of Fn14-stimulated outgrowth in adult DRG neurons suggests that Fn14 function in DRG cultures may be redundant with other outgrowth-promoting mechanisms. Other relevant pathways are likely to be numerous and may include, but not be limited to, GAP-43 and SPRR1A induction (Skene, 1989; Bonilla et al., 2002).

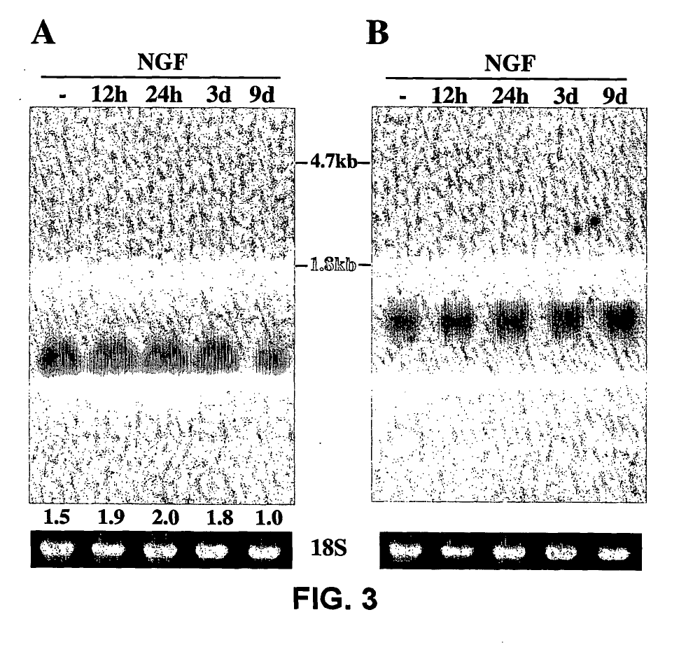

C. Fn14 receptor is linked to NGF-induced PC12 neuronal differentiation PC12 pheochromocytoma cells can differentiate under appropriate culture conditions to a neuronal phenotype and have been widely used for studies of nerve growth factor (NGF) signal transduction, neuronal survival, and neurite outgrowth. To consider the potential role of Fn14 and TWEAK during PC12 neurite growth, Fn14 and TWEAK mRNA expression levels were examined in undifferentiated and NGF-treated PC12 cells by Northern blot analysis (FIG. 3). Fn14 expression was detectable in untreated cells and was moderately increased at 12 hr after NGF addition. Peak Fn14 mRNA expression occurred after 1 d in the presence of NGF, followed by a decline after 9 d in NGF. In contrast, TWEAK mRNA expression was detected throughout differentiation and was not regulated by NGF treatment. These studies show that the Fn14 receptor is linked to NGF-induced PC12 neuronal differentiation.

D. Fn14 Induces Neurite Outgrowth in PC12 Cells

To explore a potential role for Fn14 in neurite outgrowth, wild-type and mutant forms of Fn14 were overexpressed by infection with a recombinant HSV vector (FIG. 4A). Within 20 hr of exposure to the Fn14 virus, expression of the wild-type Fn14-GFP fusion protein was observed. In the absence of any added NGF, a significant percentage of Fn14-GFP-expressing PC12 cells extend long neurntes on laminin-coated surfaces (FIG. 4B,C). Neuritogenesis was not observed in uninfected cultures or in GFP-expressing cells. This Fn14 response was more rapid and robust than the response to NGF in the same time period. Fn14 activity required both the ectodomain and the cytoplasmic domains, because expression of truncated mutant proteins had no neuritogenic effect in PC12 cells. No detectable change in cell viability was assodated with Fn14 expression in this assay, as determined by cell counts. It was clear that Fn14 overexpression can dramatically promoted neuritogenesis from PC12 cells.

PC12 cells co-express TWEAK and Fn14, therefore, studies were conducted to determine whether TWEAK could act in an autocrine manner to promote neuritogenesis. To enhance TWEAK function, purified TWEAK protein was added to the culture medium. To inhibit TWEAK function, an Fn14-Fc decoy receptor, consisting of the extracellular portion of Fn14 fused to the murine IgG1-Fc domain (Donohue etal., 2003), was added to the medium. Neither TWEAK nor Fn14-Fc promoted neurite extension from control PC12 cells (FIG. 4D). Furthermore, neither reagent altered Fn14-induced neuritogenesis. Thus, the Fn14 effect was TWEAK independent in this system. The fact that the Fn14-Fc protein had no effect on Fn14-induced neuritogenesis showed that Fn14 was acting in a ligand-independent manner.

Adult DRG neurons extend neurites much more rapidly than PC12 cells, even after NGF differentiation. The effect of overexpressing wild-type Fn14 or the potentially dominant-negative Fn14 mutant form lacking the cytoplasmic domain (Fn14-END) was examined in adult DRG neurons (FIG. 4E,F). L4-6 DRG neurons were cultured from naive animals (with low endogenous Fn14) and from animals with ipsilateral sciatic nerve lesion 1 week previously (with induced endogenous Fn14 expression). Fn14 wild-type overexpression did not significantly increase outgrowth compared with control GFP-expressing cultures. Fn14 wild-type overexpressing cells exhibited a very slight, but statistically significant, increase in outgrowth compared with Fn14-END-expressing cells. This minimal effect was significant only in naive cultures with low endogenous Fn14. As for PC12 cells, the addition of TWEAK ligand did not alter neurite outgrowth rates in adult DRG neurons (FIG. 4E).

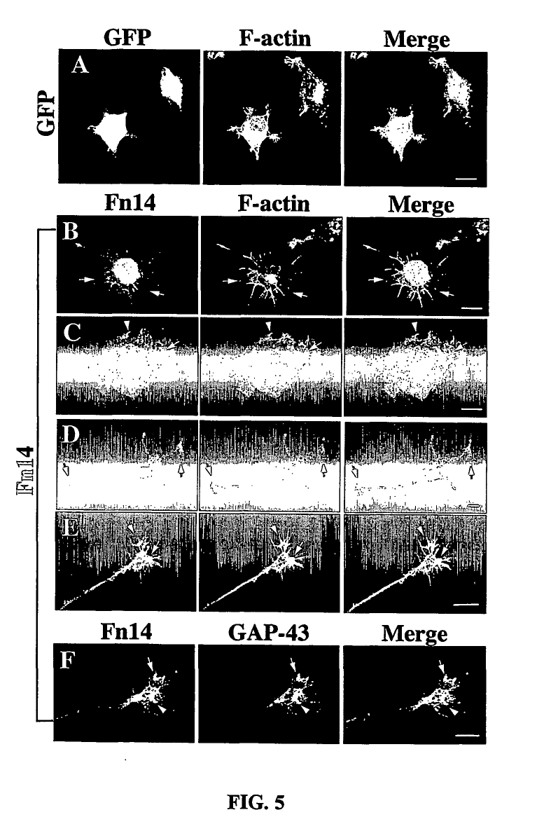

E. Fn14 induces filopodia and lamelipodia in PC12 cells The most readily apparent morphologic effect of Fn14 overexpression in PC12 cells is neuritogenesis. Closer examination, however, revealed additional morphologic changes. There was a striking increase in the presence of filopodial extensions from the cell perimeter of Fn14-overexpressing cells. Although untreated or HSV-GFP-infected PC12 cells have short filopodia at the acute angles of the cell surface (FIG. 5A), Fn14-GFP-expressing cells exhibited numerous long filopodia extending from straight cell borders as well as from angular cell borders (FIG. 5B). Concomitant with increased filopodial formation, Fn14-GFP-expressing cells showed cell spreading and lamelipodia formation (FIG. 5C). Filopodia and lamelipodia formation was also seen in extending neurites (FIG. 5D,E).

Filopodial and lamelipodial structures are rich in F-actin and their formation is dependent on microfilament rearrangements. A comparison of Fn14-GFP and F-actin localization demonstrated extensive colocalization near the cell perimeter and in filopida and lamelipodia (FIG. 5B-E). GAP43 has been localized to neuronal growth cones and has been considered a marker for regenerating neurites (Skene and Willard, 1981; Skene, 1989). Fn14-GFP-rich filopodia and lamelipodia contain abundant colocalized GAP-43 (FIG. 5F). Fn14 induction of F-actin/GAP-43rich filopodia and lamelipodia was, therefore, a precursor to neuritogenesis from PC12 cells. By these criteria, the neuritic morphology induced by NGF treatment and Fn14 overexpression share similarities at the cytoskeletal level.

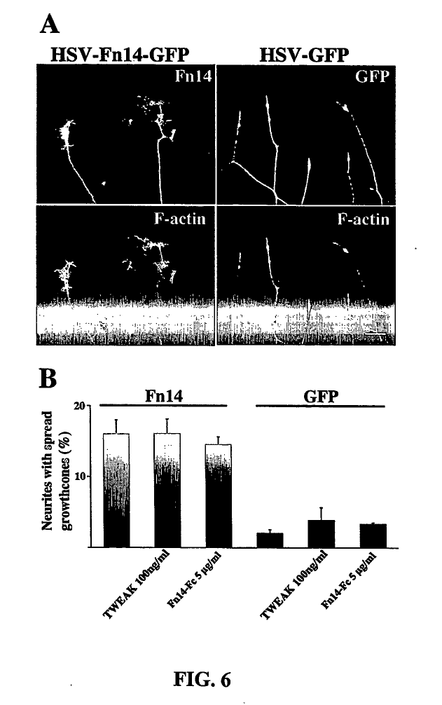

F. Fn14 promotes growth cone formation in differentiated PC12 cells The foregoing experiments demonstrate that Fn14 overexpression can promote the formation of PC12 neurites in the absence of NGF but do not reveal possible effects on neurites already established in NGF-treated cells. After 10 d of NGF treatment, PC12 cells exhibited extensive neurite outgrowth but most of the neurite tips displayed minimal lamelipodial growth cone-like specialization. To examine the effect of Fn14 overexpression on differentiated PC12 cells, cultures were infected with HSV-Fn14-GFP or HSV-GFP virus. Fn14-GFP but not GFP-expressing cells show a significant increase in the percentage of well-spread neuritic tips with growth cone widths >10 μm (FIG. 6A,B). As in the neuritogenesis assays, addition of TWEAK or Fn14-Fc did not enhance or inhibit growth cone formation (FIG. 6B). Thus, Fn14 promoted both growth cone formation and neuritogenesis by a mechanism that was ligand independent.

G. Fn14 interacts and colocalizes with Rac1 GTPase Rho family GTPases play a central role in modulating F-actin structures (Ridley and Hall, 1992; Kozma et al., 1995, 1997; Nobes and Hall, 1995; Hall, 1998) and thereby modulate neurite and growth cone motility (Mackay t at., 1995; Tanaka and Sabry, 1995; Jin and Strittmatter, 1997; Meyer and Feldman, 2002). For PC12 cells, it has been demonstrated that both activated Cdc42 and Rac lead to lamelipodia and cell spreading, whereas activated Cdc42 induces filopodia. Furthermore, effectors of Cdc42 and Rac cause PC12 neurite outgrowth (Daniels et al., 1998). Rac/Cdc42 activation Is required for neurite outgrowth in response to NGF (Chen etal., 1999; Yamaguchi etal., 2001; Yasul et al., 2001). in contrast, RhoA activation inhibits neurite outgrowth (Jalink et al., 1994; Tigyi et al., 1996; Jin and Strittmatter, 1997). The morphology of Fn14-overexpressing PC12 cells is remarkably similar to that of cells expressing activated Cdc42 or Rac1. On the basis of these observations, it was hypothesized that Fn14 modulates signal transduction by Rac1 or Cdc42, or both.

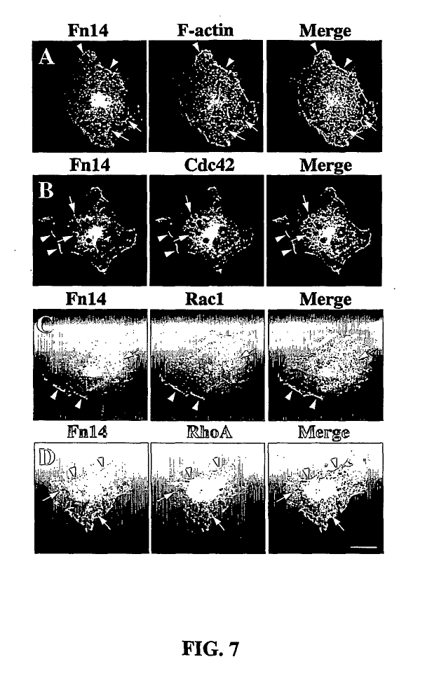

To test this hypothesis, the subcellular localization of Fn14 and Rho family GTPases were compared. COS-7 cells were cotransfected with plasmids encoding GFP-tagged Fn14 and myc-tagged Rho family GTPases. As in PC12 cells, Fn14 was enriched within leading edge ruffles and dorsal ruffles (FIG. 7). In these locations, Fn14 was colocalized with F-actin. The distribution of RhoA partially overlapped with that of Rac1, Cdc42, and Fn14 but was distinguished by Its absence from dorsal ruffles. The colocalization of Fn14 with Rac1 and Cdc42 supported the hypothesis that one or both G-proteins participate in the neuritogenic effects of Fn14.

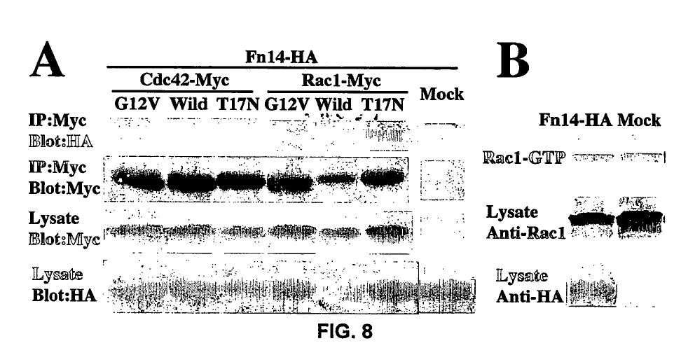

If Rac1 or Cdc42 participate in Fn14 action, then they may be present in a physical complex with Fn14. Myc epitope-tagged Rho family proteins were coexpressed with HA epitope-tagged Fn14 in HEK293T cells. Rac and Cdc42 immunoprecipitates were examined for associated Fn14. Only Rac1 immunopredpitates contained Fn14 despite equal expression of the closely related G-protein, Cdc42 (FIG. 8A). Some G-protein interactions depend on the activation state of the G-protein; however, Fn14 coimmunopredpitated equally well with wild-type, constitutively active, and dominant-negative Rac1. The equal binding to all forms of Rac raised the possibility that Fn14 does not directly alter the Rac1. activation state. Indeed, PAK pull-down assays of activated Rac1 showed no alteration of total activated Rac1. in Fn14-expressing cells (FIG. 8B). Thus, Fn14 colocalized with Rac1, physically associated with Rac1., and produced a phenotype resembling that of activated Rac1when overexpressed in PC12 cells.

The immunoprecipitation results do not distinguish between a direct and an indirect Rac1/Fn14 association. In experiments using recombinant Rac1 purified from bacterial cells, there was no detectable interaction with purified fragments of Fn14. Thus, additional cellular proteins participate in the Rac1/Fn14 complex detected in HEK293T cells.

H. Fn14-Induced Neuritogenesis Requires Rac1.

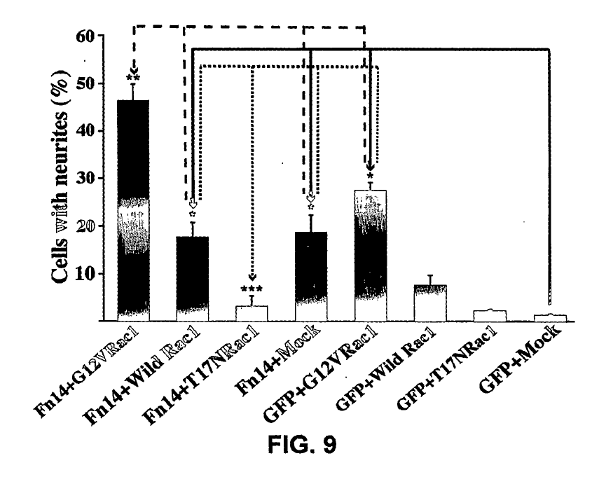

Inactivation of Rac1 should block the neuritogenesis induced by Fn14 overexpression where Fn14 effects are mediated via a protein complex that includes Rac1. To assess the role of Rac1 activity in Fn14 neuritogenesis, PC12 cells were transfected with an activated, wild-type, or dominant-negative Rac1 expression vector and replated to laminin-coated chamber slides before infection with HSV-Fn14-GFP or HSV-GFP. After 24 hr, neurite outgrowth was stimulated to a similar extent in cells co-expressing Fn14 plus wild-type Rac1 and in cells expressing activated G12VRac1 and GFP (FIG. 9). The effects of Fn14 and G12VRac1 were synergistic, leading to >50% of co-expressing cells extending neurites. In contrast, dominant-negative T17NRac1 suppressed the neuritogenic effect of Fn14. Thus, Fn14 and activated Rac1 act in the same pathway and Fn14 activity requires Rac1 function.

### IV. MYOSIN-X, SOX11, FLRT3, AND Fn14 PLAY A ROLE NEURONAL REGENERATIONS AND ARE USEFUL IN THE TREATMENT OF PAG-ASSOCIATED DISEASES AND CONDITIONS

Microarray analysis for detection of nerve regeneration-associated genes

RAGs were Identified by our microarray studies of sciatic nerve regeneration, many of which were unreported previously. Significant up-regulation of four previously unreported genes (myosin-X, SOX11, FLRT3, Fn14) was validated. Myosin-X is an unconventional myosin that Is reported to shuttle along filopodia and increase their number (Berg and Cheney, 2002). Because filopodia motility is essential for growth cone guidance, enhanced expression of myosin-X indicate that myosin-X might contribute to axonal guidance during neuronal development and regeneration. SOX11 is a transcription factor involved in neuronal differentiation (Uwanogho et al., 1995; Hargrave et al., 1997; Rex et al., 1998; Hyodo-Miura et al., 2002); however, the role of SOX11 in mature differentiated neurons has not been explored, and this study is the first report of upregulated SOX11 expression during nerve regeneration. The FLRT3 gene encodes a protein that may function as a cell surface receptor, but no ligand or functional biology is known (Lacy et al., 1999). All three of these genes are also expressed in developing DRG and have a role in nerve regeneration.

Although some of the RAGs are well-characterized and exhibit a coordinated expression in development and regeneration, Fn14 is one of a smaller group that is expressed more selectively during regeneration. The aforementioned studies demonstrate that Fn14 overexpression promotes neurite extension and growth cone formation in PC12 cells. Fn14 associates with the Rho family GTPase Rac1, and Rac1is necessary for the Fn14-induced neuronal cell effects. These data further expand the range of mechanisms capable of contributing to regenerative axon growth after injury.

Fn14 was induced in neurons during peripheral nerve regeneration but is not present during neural development in this regard, it does not fit the developmental paradigm for axonal regeneration. There is growing precedence for such a separation of these processes. Our previous studies on SPRRIA Identified another RAG that distinguished regenerative axon growth from developmental axonal extension (Bonilla et al., 2002). Similarly, JAK-STAT signaling appears to play a role selectively in regenerative growth from DRG (Liu and Snider, 2001). Although TWEAK can serve as a ligand for Fn14 in endothelial cells (Wiley et a., 2001; Harada et al., 2002; Donohue et a/, 2003), we did not find evidence for compensatory or parallel regulation of Fn14 and TWEAK during nerve regeneration. Similarly, functional studies of axon outgrowth and Fn14 argue against a role for TWEAK Thus, the function of Fn14 in this context Is ligand independent. Ugand-independent, TNF receptor-associated factor (TRAF)-dependent Fn14 activation of nuclear factor-B (NF-B) has been reported (Brown et al., 2003). Thus, the absence of a role for TVVEAK Is perhaps not surprising for these Fn14-sensitive neuronal cultures.

Fn14 overexpression promotes growth-cone formation and neurite outgrowth Although PC12 cells normally possess low levels of Fn14, overexpression of the protein induces filopodia, lamelipodia, and neuritogenesis. In NGF-differentiated cultures, Fn14 overexpression promotes growth cone formation. A direct role for Fn14 in forming filopodia, neurites, and growth cones is supported by the localization of Fn 14 protein to these structures and Its colocalization with F-actin and GAP43. These effects are clear in PC12 cells that otherwise exhibit few or no neurites and growth cones. For DRG neurons, axon growth is robust in culture, and minimal enhancement by Fn14 overexpression is observed in comparison only with cells expressing a potentially dominant-negative Fn14 truncation mutant (FIG. 4). The lack of Fn14-stimulated outgrowth in adult DRG neurons suggests that Fn14 function in DRG cultures may be redundant with other outgrowth-promoting mechanisms. Other relevant pathways are likely to be numerous and may include, but not be limited to, GAP43 and SPRRIA induction (Skene, 1989; Bonilla et al., 2002).

Rho family GTPases are known to play central roles in regulating growth cone motility and neurite extension (Mackay et al., 1995; Hall, 1998; Dickson, 2001; Meyer and Feldman, 2002). Several lines of evidence demonstrate that Fn14 promotes neuritogenesis and growth cone formation by interactions with Rac1. The two proteins colocalize and can be coimmunoprecipitated from transfected cells. In addition, activated Rac1 produces a PC12 phenotype similar to that of Fn14 overexpression, and dominant-negative Rac1blocks Fn14-induced neuritogenesis. It is notable that expression of constitutively active Rac1 in DRG neurons does not enhance axon growth (Jin and Strittmatter, 1997), a negative finding that parallels the reduced activity of Fn14 overexpression in these rapidly growing axons. For PC12 cells, an Fn14 complex with Rac1 and other unidentified proteins is capable of promoting axon outgrowth.

Fn14, like other TNF superfamily receptors, is associated via its cytoplasmic domain with adaptor proteins termed TRAFs (Wiley et al., 2001; Brown et al., 2003). Several specific TRAF isoforms are known to associate with the Fn14 cytoplasmic domain. In general, the TRAFs are known to be crucial for linking the TNF family receptors to NF-B activation. There is also some evidence, however, that TNF receptor/TRAF complexes can couple to Rho family G-proteins in non-neuronal cells (Min and Pober, 1997; Puls et al., 1999), providing one potential explanation for the neurite outgrowth effects observed here.

The effect of Fn14 expression on Rac1activation remains to be further eluddated. The Fn14 phenotype in PC12 cells resembles that of activated Rac1. Therefore, the simplest model would be that the complex formation between Fn14 and Rac1 leads to G-protein activation. Fn14 overexpression, however, does not increase the proportion of activated Rac1in the cell, and Fn14 has no binding preference for active versus inactive Rac1. Although most G-protein-regulating proteins exhibit a strong preference for either the active GTP-bound or inactive GDP-bound state, there is precedent for other proteins with nucleotide-ambivalent Rho family interactions. Drosophila PlexinB receptors bind Rho-GTP and Rho-GDP equally well, but reducing Rho gene dosage suppresses PlexinB gain-of-function phenotypes (Hu et al., 2001). The clearest example of a protein that interacts strongly with both Rac-GTP and Rac-GDP Is the Rho-GDI protein (Nomanbhoy and Cerione, 1996; Hoffrnan et al., 2000). Rho-GDI binds both states of Rho family proteins and inhibits both GTP hydrolysis and GDP release. Its in vivo activity is attributable primarily to a third activity, that of binding the geranylgeranyl moiety of the G-protein and sequestering the protein from the plasma membrane to the cytosol. Recently, several receptor systems have been shown to regulate Rho family activity indirectly by altering interactions between the G-protein and Rho-GDI (Takahashi et al., 1997; Del Pozo et al., 2002; Yamashita and Tohyama, 2003). The effect of Fn14 on Rac signaling cascades Is not fully defined. It may be that the Fn14 effect is to re-localize and concentrate Rac1 to sites more prone to lead to growth cone formation without changing its activation state. The effect would be opposite to Rho-GDI, moving the protein from the cytosol to the membrane. Alternatively, Fn14 and associated proteins may activate a small proportion of Rac1locally that is not detected under our experimental conditions. Nonetheless, Rac1Is essential for Fn14-induced neuritogenesis.

### V. REFERENCES

Araki T, Nagarajan R, Milbrandt J (2001) Identification of genes induced in peripheral nerve after injury. Expression profiling and novel gene discovery. J Biol Chem 276: 34131-34141.(Abstract/Free Full Text]
Benn S C, Perrelet D, Kato A C, Scholz J, Decosterd I, Mannion R J, Bakowska J C, Woolf C J (2002) Hsp27 upregulation and phosphorylation is required for injured sensory and motor neuron survival. Neuron 36: 45-56. [ISI][Medline]
Berg J S, Cheney R E (2002) Myosin-X Is an unconventional myosin that undergoes intrafilopodial motility. Nat Cell Biol 4: 246-250.[ISI][Medline]
Berg J S, Derfier B H, Pennisi C M, Corey D P, Cheney R E (2000) Myosin-X, a novel myosin with pleckstrin homology domains, associates with regions of dynamic actin. J Cell Sci 113: 3439-3451. [Abstract/Free Full Text]
Bonilla I E, Tanabe K, Strittmatter S M (2002) Small proline-rich repeat protein 1A is expressed by axotomized neurons and promotes axonal outgrowth. J Neurosci 22: 1303-1315. [Abstract/Free Full Text]
Bregman B S, Coumans J V, Dal H N, Kuhn P L, Lynskey J, McAtee M, Sandhu F (2002) Transplants and neurotrophic factors increase regeneration and recovery of function after spinal cord injury. Prog Brain Res 137: 257-273. [[ISI][Medline]
Brown S A, Richards C M, Hanscom H N, Feng S L, Winkles J A (2003) The Fn14 cytoplasmic tail binds tumour-necrosis-factor-receptor-associated factors 1, 2, 3 and 5 and mediates nuclear factor-kappaB activation. Biochem J 371: 395-403. [ISI][Medline]
Chen X Q, Tan I, Leung T, Lim L (1999) The myotonic dystrophy kinase-related Cdc42-binding kinase is involved in the regulation of neurite outgrowth in PC12 cells. J Biol Chem 274: 19901-19905. [Abstract/Free Full Text]
Chong M S, Woolf C J, Haque N S, Anderson P N (1999) Axonal regeneration from injured dorsal roots into the spinal cord of adult rats. J Comp Neurol 410: 42-54. [ISI][Medline]
Costigan M, Mannion R J, Kendall G, Lewis S E, Campagna J A, Coggeshall R E, Meridith-Middleton J, Tate S, Woolf C J (1998) Heat shock protein 27: developmental regulation and expression after peripheral nerve injury. J Neurosci 18: 5891-5900. [Abstract/Free Full Text]
Costigan M, Befort K, Karchewski L, Griffin R S, Da'Urso D, Allchorne A, Sitarski J, Mannion J W, Pratt R E, Woolf C J (2002) Replicate high-density rat genome oligonucleotide microarrays reveal hundreds of regulated genes in the dorsal root ganglion after peripheral nerve injury. BMC Neurosci 3: 16. [Medline]
Coumans J V, Un T T, Dal H N, MacArthur L, McAtee M, Nash C, Bregman B S (2001) Axonal regeneration and functional recovery after complete spinal cord transection in rats by delayed treatment with transplants and neurotrophins. J Neurosci 21: 9334-9344. [Abstract[Free Full Text]
Daniels R H, Hall P S, Bokoch G M (1998) Membrane targeting of p21-activated kinase 1 (PAK1) induces neurite outgrowth from PC12 cells. EMBO J 17: 754-764. (Abstrac/Free Full Text]
Del Pozo M A, Kiosses W B, Alderson N B, Meller N, Hahn K M, Schwartz M A (2002) integrins regulate GTP-Rac localized effector interactions through dissociation of Rho-GDI. Nat Cell Biol 4: 232-239. [ISI][Medline]
Desplat-Jego S, Varriale S, Creidy R, Terra R, Bernard D, Khrestchatisky M, Izui S, Chicheportiche Y, Boucraut J (2002) TWEAK is expressed by glial cells, induces astrocyte proliferation and increases EAE severity. J Neuroimmunol 133: 116-123.[ISI][Medline]
Dickson B J (2001) Rho GTPases in growth cone guidance. Curr Opin Neurobiol 11: 103-110. [ISI][Medline]
Donohue P J, Richards C M, Brown S A, Hanscom H N, Buschman J, Thangada S, Hla T, Williams M S, Winkles J A (2003) TWEAK is an endothelial cell growth and chemotactic factor that also potentiates FGF-2 and VEGF-A mitogenic activity. Arterioscier Thromb Vasc Biol 23: 594-600. [Abstract/Free Full Text]
Fawcett J W, Asher R A (1999) The glial scar and central nervous system repair. Brain Res Bull 49: 377-391. [ISI][Medline]
Foumier A E, Strittmatter S M (2001) Repulsive factors and axon regeneration in the CNS. Curr Opin Neurobiol 11: 89-94. [ISI][Medline]
Funakoshi H, Frisen J, Barbany G, Timmusk T, Zachrisson O, Verge V M, Persson H (1993) Differential expression of mRNAs for neurotrophins and their receptors after axotomy of the sciatic nerve. J Cell Biol 123: 455-465. [Abstract]
Goldberg J L, Klassen M P, Hua Y, Barres B A (2002) Amacrine-signaled loss of intrinsic axon growth ability by retinal ganglion cells. Science 296: 1860-1864. [Abstract/Free Full Text]
GrandPre T, Nakamura F, Vartanian T, Strittmatter S M (2000) Identification of the Nogo inhibitor of axon regeneration as a Reticulon protein. Nature 403: 439-444.[ISI][Medline]
GrandPre T, Li S, Strittmatter S M (2002) Nogo-66 receptor antagonist peptide promotes axonal regeneration. Nature 417: 547-551. [ISI][Medline]
Hall A (1998) Rho GTPases and the actin cytoskeleton. Science 279: 509-514. [Abstract/Free Full Text]
Harada N, Nakcayama M, Nalkano H, Fuluchi Y, Yagita H, Okumura K (2002) Pro-inflammatory effect of TWEAK/Fn14 interaction on human umbilical vein endothelial cells. Biochem Biophys Res Commun 299: 488-493. [ISI][Medline]
Hargrave M, Wright E, Kun J, Emery J, Cooper L, Koopman P (1997) Expression of the Sox11 gene in mouse embryos suggests roles in neuronal maturation and epithelio-mesenchymal induction. Dev Dyn 210: 79-86. [ISI][Medline]
Hoffman G R, Nassar N, Cerione R A (2000) Structure of the Rho family GTP-binding protein Cdc42 in complex with the multifunctional regulator RhoGDI. Cell 100: 345-356.[ISI][Medline]
Hu H, Marton T F, Goodman C S (2001) Plexin B mediates axon guidance in Drosophila by simultaneously inhibiting active Rac and enhancing RhoA signaling. Neuron 32: 39-51.[ISI][Medline]
Hyodo-Miura J, Urushlyama S, Nagai S, Nishita M, Ueno N, Shibuya H (2002) involvement of NLK and Sox11 in neural induction in Xenopus development Genes Cells 7: 487-496. [ISI][Medline]
Jalink K, van Corven E J, Hengeveld T, Morii N, Narumiya S, Moolenaar W H (1994) inhibition of lysophosphatidate- and thrombin-induced neurite retraction and neuronal cell rounding by ADP ribosylation of the small GTP-binding protein Rho. J Cell Biol 126: 801-810.[Abstract]
Jay P, Goze C, Marsollier C, Taviaux S, Hardelin J P, Koopman P. Berta P (I995) The human SOX11 gene: cloning, chromosomal assignment and tissue expression. Genomics 29: 541-545. [ISI][Medline]
Jin Z, Strittmafter S M (1997) Rac1 mediates collapsin-1-induced growth cone collapse. J Neurosci 17: 6256-6263. [Abstract/Free Full Text]
Kiryu S, Yao G L, Morita N, Kato H, Kiyama H (1995) Nerve injury enhances rat neuronal glutamate transporter expression: identification by differential display PCR. J Neurosci 15: 7872-7878. [Abstract]
Kozma R, Ahmed S, Best A, Lim L (1995) The Ras-related protein Cdc42Hs and bradykinin promote formation of peripheral actin microspikes and filopodia in Swiss 3T3 fibroblasts. Mol Cell Biol 15: 1942-1952. [Abstract]
Kozma R, Samer S, Ahmed S, Lim L (1997) Rho family GTPases and neuronal growth cone remodeling: relationship between increased complexity induced by Cdc42Hs, Rac1, and acetylcholine and collapse induced by RhoA and lysophosphatidic acid. Mol Cell Biol 17: 1201-1211. [Abstract]
Lacy S E, Bonnemann C G, Buzney E A, Kunkel L M (1999) Identification of FLRT1, FLRT2, and FLRT3: a novel family of transmembrane leucine-rich repeat proteins. Genomics 62: 417-426. [ISI][Medline]
Landry M, Holmberg K, Zhang X, Hokfelt T (2000) Effect of axotomy on expression of NPY, galanin, and NPY Y1 and Y2 receptors in dorsal root ganglia and the superior cervical ganglion studied with double-labeling in situ hybridization and immunohistochemistry. Exp Neurol 162: 361-384. [ISI][Medline]
Lewis S E, Mannion R J, White F A, Coggeshall R E, Beggs S, Costigan M, Martin J L, Dillmann W H, Woolf C J (1999) A role for HSP27 in sensory neuron survival. J Neurosci 19: 8945-8953. [Abstrac/Free Full Text]
Uu R Y, Snider W D (2001) Different signaling pathways mediate regenerative versus developmental sensory axon growth. J Neurosci 21: RC164(1-5).[Abstract/Free Full Text]
Lynch C N, Wang Y C, Lund J K, Chen Y W, Leal J A, Wiley S R (1999) TWEAK induces angiogenesis and proliferation of endothelial cells. J Biol Chem 274: 8455-8459. [AbstracVFree Full Text]
Mackay D J, Nobes C D, Hall A (1995) The Rho's progress: a potential role during neuritogenesis for the Rho family of GTPases. Trends Neurosci 18: 496-501. [lSI][Medline]
Mattsson P, Morgan B P, Svensson M (1998) Complement activation and CD59 expression in the motor facial nudeus following intracranial transection of the facial nerve in the adult rat. J Neuroimmunol 91: 180-1 89. [ISI][Medline]
McKerracher L, David S, Jackson D L, Kotis V, Dunn R J, Braun P E (1994) Identfication of myelin-associated glycoprotein as a major myelin-derived inhibitor of neurite growth. Neuron 13: 805-811.[ISI][Medline]
Meighan-Mantha R L, Hsu D K, Guo Y, Brown S A, Feng S L, Peifley K A, Alberts G F, Copeland N G, Gilbert D J, Jenkins N A, Richards C M, Winkles J A (1999) The mitogen-inducible Fn14 gene encodes a type I transmembrane protein that modulates fibroblast adhesion and migration. J Biol Chem 274: 33166-33176.[Abstract/Free Full Text]
Meyer G, Feldman E L (2002) Signaling mechanisms that regulate actin-based motility processes in the nervous system. J Neurochem 83: 490-503. [Abstrac/Free Full Text]
Min W, Pober J S (1997) TNF initiates E-selectin transcription in human endothelial cells through parallel TRAF-NF-kappa B and TRAF-RAC/CDC42-JNK-cJun/ATF2 pathways. J immunol 159: 3508-3518. [Abstract]
Moskowitz P F, Oblinger M M (1995) Sensory neurons selectively upregulate synthesis and transport of the III-tubulin protein during axonal regeneration. J Neurosci 15: 1545-1555. [Abstract]
Muma N A, Hoffman P N, Slunt H H, Applegate M D, Lieberburg I, Price D L (1990) Alterations in levels of mRNAs coding for neurofilament protein subunits during regeneration. Exp Neurol 107: 230-235. [ISI][Medline]
Nakamura F, Tanaka M, Takahashi T, Kalb R G, Striftmafter S M (1998) Neuropilin-1 extracelfular domains mediate semaphorin D/III-induced growth cone collapse. Neuron 21: 1093-1100. [ISI][Medline]
Neumann S, Woolf C J (1999) Regeneration of dorsal column fibers into and beyond the lesion site following adult spinal cord injury. Neuron 23: 83-91. [ISI][Medline]
Niederost B P, Zimmermann D R, Schwab M E, Bandtlow C E (1999) Bovine CNS myelin contains neurite growth-inhibitory activity associated with chondroitin sulfate proteoglycans. J Neurosci 19: 8979-8989. [Abstract/Free Full Text]
Nobes C D, Hall A (1995) Rho, rac, and cdc42 GTPases regulate the assembly of multimolecular focal complexes associated with actin stress fibers, lamellipodia, and filopodia. Cell 81: 5342. [ISI][Medline]
Nomanbhoy T K, Cerione R (1996) Characterization of the interaction between RhoGDI and Cdc42Hs using fluorescence spectroscopy. J Biol Chem 271: 10004-10009. [Abstract/Free Full Text]
Puls A, Eliopoulos A G, Nobes C D, Bridges T, Young L S, Hall A (1999) Activation of the small GTPase Cdr42 by the inflammatory cytokines TNF(alpha) and IL-1, and by the Epstein-Barr virus transforming protein LMP1. J Cell Sci 112: 2983-2992. [Abstract[Free Full Text]
Qiu J, Cal D, Filbin M T (2002a) A role for CAMP in regeneration during development and after injury. Prog Brain Res 137: 381-387. [ISI][Medline]
Qiu J, Cal D, Dal H, McAtee M, Hoffman P N, Bregman B S, Filbin M T (2002b) Spinal axon regeneration induced by elevation of cyclic AMP. Neuron 34: 895-903. [ISI][Medline]
Rex M, Church R, Tointon K, Ichihashi R M, Mokhtar S, Uwanogho D, Sharpe P T, Scotting P J (1998) Granule cell development in the cerebellum is punctuated by changes in Sox gene expression. Brain Res Mol Brain Res 55: 28-34. [ISI][Medline]
Richardson P M, McGuinness U M, Aguayo A J (1980) Axons from CNS neurons regenerate into PNS grafts. Nature 284: 264-265. [ISI][Medline]
Ridley A J, Hall A (1992) The small GTP-binding protein rho regulates the assembly of focal adhesions and actin stress fibers in response to growth factors. Cell 70: 389-399. [ISI][Medline]
Skene J H (1989) Axonal growth-associated proteins. Annu Rev Neurosci 12: 127-156. [ISI][Medline]
Skene J H, Willard M (1981) Axonally transported proteins associated with axon growth in rabbit central and peripheral nervous systems. J Cell Biol 89: 96-103. [ISI][Medline]
Takahashi K, Sasaki T, Mammoto A, Takaishi K, Kameyama T, Tsukita S, Takai Y (1997) Direct interaction of the Rho GDP dissociation inhibitor with ezrin/radixin/moesin initiates the activation of the Rho small G protein. J Biol Chem 272: 23371-23375. [Abstract/Free Full Text]
Takahashi T, Nakamura F, Jin Z, Kalb R G, Striftmatter S M (1 998) Semaphorins A and E act as antagonists of neuropilin-1 and agonists of neuropilin-2 receptors. Nat Neurosci 1: 487-493. [ISI][Medline]
Takahashi T, Foumier A, Nakamura F, Wang L H, Murakami Y, Kalb R G, Fujisawa H, Strittmatter S M (1999) Plexin-neuropilin-1 complexes form functional semaphorin-3A receptors. Cell 99: 59-69. [ISI][Medline]
Tanabe K, Nakagomi S, Kiryu-Seo S, Namikawa K, Imai Y, Ochi T, Tohyama M, Kiyama H (1999) Expressed-sequence-tag approach to identify differentially expressed genes following peripheral nerve axotomy. Brain Res Mol Brain Res 64: 34-40. [ISI][Medline]
Tanabe K, Tachibana T, Yamashita T, Che Y H, Yoneda Y, Ochi T, Tohyama M, Yoshikawa H, Kiyama H (2000) The small GTP-binding protein TC10 promotes nerve elongation in neuronal cells, and its expression is induced during nerve regeneration in rats. J Neurosci 20: 4138-4144. [Abstract/Free Full Text]
Tanaka E, Sabry J (1995) Making the connection: cytoskeletal rearrangements during growth cone guidance. Cell 83: 171-176. [ISI][Medline]
Tigyi G, Fischer D J, Sebok A, Yang C, Dyer D L, Miledi R (1996) Lysophosphatidic acid-induced neurite retraction in PC12 cells: control by phosphoinos!Ude-Ca2+signaling and Rho. J Neurochem 66: 537-548. [Abstract]
Tsujino H, Kondo E, Fukuoka T, Dal Y, Tokunaga A, Miki K, Yonenobu K, Ochi T, Noguchi K (2000) Activating transcription factor 3 (ATF3) induction by axotomy in sensory and motoneurons: a novel neuronal marker of nerve injury. Mol Cell Neurosci 15: 170-182. [ISI][Medline]
Uwanogho D, Rex M, Cartwright E J, Pearl G, Healy C, Scotting P J, Sharpe P T (1995) Embryonic expression of the chicken Sox2, Sox3 and Sox11 genes suggests an interactive role in neuronal development. Mech Dev 49: 23-36. [ISI][Medline]
Villar M J, Cortes R, Theodorsson E, Wiesenfeld-Hallin Z, Schalling M, Fahrenkrug J, Emson P C, Hokfelt T (1989) Neuropeptide expression in rat dorsal root ganglion cells and spinal cord after peripheral nerve injury with special reference to galanin. Neuroscience 33: 587-604. [ISI][Medline]
Wakisaka S, Kajander K C, Bennett G J (1991) increased neuropeptide Y (NPY)-like immunoreactivity in rat sensory neurons following penpheral axotomy. Neurosci Lett 124: 200-203. [ISI[Medline]
Wang K C, Koprivica V, Kim J A, Sivasankaran R, Guo Y, Neve R L, He Z (2002) Oligodendrocyte-myelin glycoprotein is a Nogo receptor ligand that inhibits neurite outgrowth. Nature 417: 941-944. [ISI][Medline]
Wiley S R, Cassiano L, Lofton T, Davis-Smith T, Winkles J A, Lindner V, Liu H, Daniel T O, Smith C A, Fanslow W C (2001) A novel TNF receptor family member binds TWEAK and is implicated in angiogenesis. Immunity 15: 837-846. [ISI][Medline]
Yamaguchi Y, Katoh H, Yasui H, Mori K, Negishi M (2001) RhoA inhibits the nerve growth factor-induced Rac1 activation through Rho-associated kinase-dependent pathway. J Biol Chem 276: 18977-18983. [Abstract/Free Full Text]
Yamashita T, Tohyama M (2003) The p75 receptor acts as a displacement factor that releases Rho from Rho-GDI. Nat Neuroscd 6: 461-467. [ISI][Medline]
Yasui H, Katoh H, Yamaguchi Y, Aoid J, Fujita H, Mori K, Negishi M (2001) Differential responses to nerve growth factor and epidermal growth factor in neurite outgrowth of PCi2 cells are determined by Rac1. activation systems. J Biol Chem 276: 15298-15305. [Abstrac/Free Full Text]
Yonezawa S, Kimura A, Koshiba S, Masaki S, Ono T, Hanai A, Sonta S, Kageyama T, Takahashi T, Moriyama A (2000) Mouse myosin X: molecular architecture and tissue expression as revealed by northern blot and In situ hybridization analyses. Biochem Biophys Res Commun 271: 526-533. [ISI][Medline]
Zhang X, Bao L, Xu Z Q, Diez M, Frey P, Hokfelt T (1996) Peripheral axotomy induces increased expression of neurotensin in large neurons in rat lumbar dorsal root ganglia. Neurosci Res 25: 359-369.

### Example 2

### Systems and Methods for Structure-Based Rational Drug Design

RAG compounds can be designed and refined, in part, based on structural models such as x-ray crystallography and nuclear magnetic resonance, and the following references (all incorporated herein in there entirety) are suitable models for the crystallization, preparation and structural analysis of the RAG compounds disclosed herein. Methods of structure-based drug design using crystalline polypeptides are described in at least U.S. Pat. Nos 6,329,184 and 6,403,330 both to Uppenberg. Methods for using x-ray topography and diffractometry to improve protein crystal growth are described in U.S. Pat. No. 6,468,346 to Arnowitz, et al. Methods and apparatus for automatically selecting Bragg reflections, and systems for automatically determining crystallographic orientation are described by U.S. Pat. No. 6,198,796 to Yokoyama, et al., Methods for the preparation and labeling of proteins for NMR with ¹³C, ¹⁵N, and ²H for structural determinations is described in U.S. Pat. No. 6,376,253 to Anderson, et al. NMR spectroscopy of large or complex proteins is described in U.S. Pat. No. 6,198,281 to Wand, et al., Use of nuclear magnetic resonance to design ligands to target biomolecules Is described in U.S. Pat. No. 5,989,827 to Fesik, et al.

The process of rational drug design of RAG protein mimetics with nuclear magnetic resonance includes the steps of Identifying a candidate RAG compound that is a potential ligand to the target molecule (such as a RAG receptor) using two-dimensional ¹⁵N/¹H NMR correlation spectroscopy; b) forming a binary complex by binding the candidate RAG compound to the target molecule, c) determining the three dimensional structure of the binary complex and thus the spatial orientation of the candidate RAG compound on the target molecule. The process of rational drug design of RAG protein mimetics with x-ray crystallography is accomplished in a similar manner, but structural data is first obtained by forming crystals of the candidate RAG compound that is a potential ligand to the target molecule (or co-crystals of the complex), and obtaining a data set of the atomic reflections after x-ray irradiation. These techniques are known to those skilled in the art in view of the teachings provided herein.

Refinements to the candidate RAG compound are then made to increase the affinity of the candidate RAG compound for the target molecule. Refinements include constraining and cyclizing the RAG compound or incorporation of non-classical amino acids that induce conformational constraints. A constrained, cyclic or rigidized RAG compound may be prepared synthetically, provided that in at least two positions in the sequence of the RAG compound, an amino acid or amino acid analog is inserted that provides a chemical functional group capable of cross-linking to constrain, cyclise or rigidize the RAG compound after treatment to form the crosslink. Cyclization will be favored when a tum-inducing amino acid is incorporated. Examples of amino acids capable of crosslinking a RAG compound are cysteine to form disulfides, aspartic acid to form a lactone or a lactam, and a chelator such as gamma-carboxyl glutamic acid (Gla) (Bachem) to chelate a transition metal and form a cross-link. Protected gamma.-carboxyl glutamic acid may be prepared by modifying the synthesis described by Zee-Cheng and Olson (Biophys. Biochem. Res. Commun., 94:1128-1132 (1980)). A RAG compound in which the peptide sequence comprises at least two amino adds capable of crosslinking may be treated, e.g., by oxidation of cysteine residues to form a disulfide or addition of a meal ion to form a chelate, so as to crosslink the peptide and form a constrained, cyclic or rigidized RAG compound.

Non-classical amino acids may be incorporated in the RAG compound in order to introduce particular conformational motifs, for example but not limited to 1 ,2,3,4-tetrahydroisoquinolne-3-carboxylate (Kazmierski et al., J. Am. Chem. Soc., 113:2275-2283 (1991)); (2S,3S)-methyl-phenylalanine, (2S,3R)-methyl-phenylalanine, (2R,3S)methyl-phenylalanine and (2R,3R)-methyl-phenylalanine (Kazmierski and Hruby, Tetrahedron Lett. (1991)); 2-aminotetrahydronaphthalene-2-carboxylic acid (Landis, Ph.D. Thesis, University of Arizona (1989)); hydroxy-1,2,3,4-tetrahydroisoquinoline-3-carboxylate (Miyake et al., J. Takeda Res. Labs, 43:53-76 (1989)); beta-carboline (D and L) (Kazmierski, Ph.D. Thesis, University of Arizona (1988)); HIC (histidine isoquinoline carboxylic acid) (Zechel et al., Int. J. Pep. Protein Res., 43 (1991)); and HIC (histidine cyclic urea). Amino acid analogs and peptidomimetics may be incorporated into a peptide to induce or favor specific secondary structures, including but not limited to: LL-Acp-(LL-3-amino-2-propenidone-6-carboxylic acid), a beta-turn inducing dipeptide analog (Kemp et al., J. Org. Chem. 50:5834-5838 (1985)); beta-sheet inducing analogs (Kemp et al., Tetrahedron Lett. 29:5081-5082 (1988)); beta-turn including analogs (Kemp etat, Tetrahedron Lett., 29:5057-5060 (1988)); helix Inducing analogs (Kemp et al., Tetrahedron Lett., 29:4935-4938 (1988)); gamma-tum inducing analogs (Kemp et al., J. Org. Chem. 54:109:115 (1989)); and analogs provided by the following references: Nagai and Sato, Tetrahedron Left., 26:647;14 650 (1985); DiMalo et al., J. Chem. Soc. Perkin Trans. p. 1687 (1989); also a Gly-Ala turn analog (Kahn et a/, Tetrahedron Lett., 30:2317 (1989)); amide bond isoetere (Jones et al., Tetrahedron Lett., 29:3853-3856 (1988)) tretazol (Zabrocki et al., J. Am. Chem. Soc. 110:5875-5880 (1988)); DTC (Samanen et al., Int. J. Protein Pep. Res., 35:501:509 (1990)); and analogs taught in Olson etal., J. Am. Chem. Sci., 112:323-333 (1990) and Garvey et al., J. Org. Chem., 56:436 (1990). ConformaUonally-restricted mimetics of beta turns and beta bulges, and peptides containing them, are described in U.S. Pat. No. 5,440,013, Issued Aug. 8, 1995 to Kahn.

Once the three-dimensional structure of a RAG compound (or a refinement of the same) is determined, Its therapeutic potential (as an antagonist or agonist) can be examined through the use of computer modeling using a docking program such as GRAM, DOCK, or AUTODOCK. Computer programs that can be used to aid in solving the three-dimensional structure of the RAG compound and binding complexes thereof include QUANTA, CHARMM, INSIGHT, SYBYL, MACROMODE, and ICM, MOLMOL, RASMOL, AND GRASP (Kraulis, J. Appl. Crystallogr. 24:946-950 (1991)). Most if not all of these programs and others as well can be also obtained from the World Wide Web through the internet. The rational design of RAG compounds can include computer fitUng of potential agents to the RAG compound to ascertain how well the shape and the chemical structure of the modified RAG compound will complement or interfere with the interaction between the RAG compound and its ligand. Computer programs can also be employed to estimate the attraction, repulsion, and steric hindrance of the potential therapeutic RAG compound to the RAG binding site of a RAG target molecule, for example. Generally the tighter the fit (e.g., the lower the steric hindrance, and/or the greater the attractive force) the more potent the potential therapeutic RAG compound will be since these properties are consistent with a tighter binding constraint.

Initially a potential therapeutic RAG compound can be obtained by screening a random peptide library produced by recombinant bacteriophage for example, (Scoft and Smith, Science, 249:386-390 (1990); Cwirla et al., Proc. Natl. Acad. Sci., 87:6378-6382 (1990); Devlin et al., Science, 249:404-406 (1990)) or a chemical library. A candidate therapeutic RAG compound selected in this manner Is then systematically modified by computer modeling programs until one or more promising potential therapeutic RAG compounds are identified. Such analysis has been shown to be effective in the development of HIV protease inhibitors (Lam et al., Science 263:380-384 (1994); Wlodawer et al., Ann. Rev. Biochem. 62:543-585 (1993); Appelt, Perspectives in Drug Discovery and Design 1:2348 (1993); Erickson, Perspectives in Drug Discovery and Design 1: 109-128 (1993)). A computer-based method for classifying and producing analogs of polypeptides and can be found at PCT Publication WO/02/37313 to Keck, and is useful for the selection of RAG compounds as described herein.

Such computer modeling allows the selection of a finite number of rational chemical modifications, as opposed to the countless number of essentially random chemical modifications that could be made, any of which any one might lead to a useful drug. Each chemical modification requires additional chemical steps, which while being reasonable for the synthesis of a finite number of compounds, quickly becomes overwhelming if all possible modifications needed to be synthesized. Thus through the use of the three-dimensional structural analysis disclosed herein and computer modeling, a large number of these candidate RAG compounds can be rapidly screened, and a few likely candidate therapeutic RAG compounds can be determined without the laborious synthesis of untold numbers of RAG compounds.

The candidate therapeutic RAG compounds can then be tested in any standard binding assay (including in high throughput binding assays) for its ability to bind to a RAG, RAG polypeptide or fragment thereof. Alternatively the potential drug can be tested for its ability to modulate (either inhibit or stimulate) the biological activity of a RAG. When a suitable potential drug Is identified, a second structural analysis can optionally be performed on the binding complex formed between the ligand and the candidate therapeutic RAG compound. For all of the screening assays described herein further refinements to the structure of the candidate RAG therapeutic compound will generally be necessary and can be made by the successive iterations of any and/or all of the steps provided by the particular drug screening assay, including further structural analysis by x-ray crystallography or NMR, for example.

### Example 3

### An In vitro Assay for Biological Activity

For analysis of the biological activity of RAG test compound in vitro, DRG neurons are removed from the L4-6 level from 8- to 10-week-old mice with or without sciatic nerve crush injuries 1 week previously. Neurons are dissociated, cultured, and infected with HSV preparations as described previously (Bonilla et al., 2002). Cells are contacted, e.g., exogenous application or transfection, with increasing concentrations (e.g., 1 pmol10 mol test compound per well) of RAG compound. After 24 hr in culture, cells are fixed, and neurite outgrowth scored as described (Bonilla et al., 2002). An increase in the level of neurite growth of cells contacted with RAG compound compared to level of neurite growth of cells observed in the absence of contact with RAG compound indicates that the RAG compound is a therapeutic compound useful to promote nerve regeneration and growth. A decrease in the level of neurite growth of cells contacted with RAG compound compared to the level of neurite growth of cells observed in the absence of contact with RAG compound indicates that the RAG compound is a therapeutic compound useful to inhibit nerve regeneration and growth.

### Example 4

### An In vivo Assay for Biological Activity

For analysis of the effect of RAG test compound on axon regeneration in vivo, DRG axons are transected in the dorsal columns on both sides of the spinal cord, at the level of the cervico-thoracic junction (>4 weeks). A segment of sciatic nerve on one side is resected and grafted into the spinal cord lesion site to provide injured axons with an optimal environment for regrowth (Richardson et al., Nature 309:791 (1984); Richardson et al., J. Neurocytol. 15: 585 (1986)). The resection produces a peripheral nerve injury that affects DRG neurons on the same side as the lesion, but leaves the contralateral ganglia uninjured except for the spinal cord lesion. RAG test compound is injected daily into the region of the graft of test subjects. An equivalent volume of vehicle alone Is injected daily into the region of the graft of control subjects. After 1 week to 4 months, the fluorescent axonal tracer dil is introduced into the distal end of the nerve grafts to visualize neurons that regenerated their axons at lest 5 mm into the graft. After another 5 days, the animals are perfused transcardially with 4% paraformaldehyde, and the dorsal root ganglia removed and post-fixed in 30% sucrose. Thirty micron cryostat tissue sections are prepared and evaluated under fluorescent microscopy. Fluorescently labeled cells are counted.

An increase in the number of labeled nerve cells in the graft region of test animals that received RAG compound compared to number of labeled of nerve cells observed in the graft region of control animals receiving the control vehicle indicates that the RAG compound is a therapeutic compound useful to promote nerve regeneration and growth. A decrease in the number of labeled nerve cells in the graft region of test animals that received RAG compound compared to number of labeled of nerve cells observed in the graft region of control animals receiving the control vehicle indicates that the RAG compound Is a therapeutic compound useful to inhibit nerve regeneration and growth.

### Equivalents

From the foregoing detailed description of the specific embodiments of the invention, it should be apparent that unique bioactive peptides have been described. Although particular embodiments have been disclosed herein in detail, this has been done by way of example for purposes of illustration only, and is not intended to be limiting with respect to the scope of the appended claims which follow. In particular, it is contemplated by the inventor that various substitutions, alterations, and modifications may be made to the invention without departing from the spirit and scope of the invention as defined by the claims.

What is claimed is:

1. A method of regenerating neurons comprising administering to a subject in need thereof a RAG compound that modulates the activity of an Fn14 polypeptide (RAG ID NO:6) in amounts effective to cause neuronal regeneration.

2. A method according to claim 1, wherein the RAG compound is selected from a group consisting of: a polypeptide, peptde, polynucleotide, antiRAG antbody, and small molecule.

3. A method according to claim 1, wherein the subject Is a human.

4. A method according to claim 1, wherein the RAG compound increases the activity of the Fn14 polypeptide (RAG ID NO:6).

5. A method according to claim 4, wherein the RAG compound Is an Fn14 agonist.

6. A method according to claim 4, wherein the RAG compound Is a TWEAK polypeptide, homolog, or fragment thereof.

7. A method according to claim 1, wherein the RAG compound increases the expression of the Fn14 polypeptide (RAG ID NO:6).

8. A method according to claim 1, wherein the RAG compound decreases the activity of the Fn14 polypeptide (RAG ID NO:6).

9. A method according to claim 8, wherein the RAG compound is an Fn14 antagonist.

10. A method according to claim 1, wherein the RAG compound decreases the expression of the Fn14 polypeptide (RAG ID NO:6).

11. A method of inhibiting neuron growth comprising administering to a subject in need thereof a RAG compound that modulates the activity of an Fn14 polypeptide (RAG ID NO:6) in amounts effective to cause inhibition of neuronal growth.

12. A method according to claim 11, wherein the RAG compound is selected from a group consisting of a polypeptide, peptide, polynucleotide, anti-RAG antibody, and small molecule.

13. A method according to claim 11, wherein the subject is a human.

14. A method according to claim 11, wherein the RAG compound increases the activity of the Fn14 polypeptide (RAG ID NO:6).

15. A method according to claim 14, wherein the RAG compound is an Fn14 agonist.

16. A method according to claim 11, wherein the RAG compound increases the expression of the Fn14 polypeptide (RAG ID NO:6).

17. A method according to claim 11, wherein the RAG compound decreases the activity of the Fn14 polypeptide (RAG ID NO:6).

18. A method according to claim 17, wherein the RAG compound is an Fn14 antagonist

19. A method according to claim 11, wherein the RAG compound decreases the expression of the Fn14 polypeptide (RAG ID NO:6).

20. A method of regenerating neurons comprising administering to a subject in need thereof an effective amount of an Fn14 polypeptide (RAG ID NO:6).

21. A method according to claim 20, wherein a composition that upregulates the Fn14 polypeptide (RAG ID NO:6) is administered to the subject.

22. A method according to claim 20, wherein a composition that down-regulates the Fn14 polypeptide (RAG ID NO:6) is administered to the subject.

23. A method according to claim 21, wherein the composition comprises a vector that delivers an Fn14 polynucleotide (RAG ID NO:6).

24. A method according to claim 22, wherein the composition comprises a vector that delivers an Fn14 polynucleotide (RAG ID NO:6).

25. A method according to claim 20, wherein the subject Is a human.

26. The use of a compound for the manufacture of a medicament for treatment of a RAG-associated disorder, wherein the compound is an Fn14 polypeptide (RAG ID NO:6).

27. A use according to claim 26, wherein the RAG-associated disorder Is selected from the group consisting of brain trauma, spinal cord trauma, stroke, cerebral palsy, multiple sclerosis, Parkinson's disease, Alzheimer's disease, ALS, stroke, peripheral neuropathies, brain cancer, neuroblastoma, and reinnervation of transplanted tissue.

28. A method of treating or preventing a RAG-associated disorder, the method comprising administering to a subject in which such treatment or prevention Is desired an Fn14 polypeptide (RAG ID NO:6) in an amount sufficient to treat or prevent the RAG-associated disorder.

29. A method according to claim 28, wherein the RAG-associated disorder is selected from the group consisting of: brain trauma, spinal cord trauma, stroke, cerebral palsy, multiple sclerosis, Parkinson's disease, Alzheimer's disease, ALS, stroke, peripheral neuropathies, brain cancer, neuroblastoma, and reinnervation of transplanted tissue.

30. A method according to claim 28, wherein the subject is a human.

31. A method of treating or preventing a RAG-associated disorder, the method comprising administering to a subject in which such treatment or prevention is desired a polynucleotide encoding an Fn14 polypeptide (RAG ID NO:6) in an amount sufficient to treat or prevent the RAG-associated disorder in the subject.

32. A method according to claim 31, wherein the subject is a human.

33. A method according to claim 31, wherein the RAG-associated disorder Is selected from the group consisting of RAG-associated disorder is selected from the group consisting of brain trauma, spinal cord trauma, stroke, cerebral palsy, multiple sclerosis, Parkinson's disease, Alzheimer's disease, ALS, stroke, peripheral neuropathies, brain cancer, neuroblastoma, and reinnervation of transplanted tissue.

34. A method of treating a pathological state in a mammal, the method comprising administering to the mammal a compound in an amount that is sufficient to alleviate the pathological state, wherein the compound is a compound having an amino acid sequence at least 90% identical to a compound comprising an Fn14 polypeptide (RAG ID NO:6).

35. A method of treating a RAG-associated disorder in a mammal, the method comprising administering to the mammal at least one compound which modulates the expression or activity of an Fn14 polypeptide (RAG ID NO:6).

36. A method according to claim 35, wherein the RAG-associated disorder is selected from the group consisting of: brain trauma, spinal cord trauma, stroke, cerebral palsy, multiple sclerosis, Parkinson's disease, Alzheimer's disease, ALS, stroke, peripheral neuropathies, brain cancer, neuroblastoma, and reinnervation of transplanted tissue.

37. A method of identifying a compound which binds to an Fn14 polypeptide (RAG ID NO:6), the method comprising the steps of:
a) providing a candidate compound;
b) contacting the candidate compound with the Fn14 polypeptide under conditions which a complex is formed between the candidate compound and the Fn14 polypeptide;
c) Incubating the complex under conditions where co-crystals of the complex form;
d) determining the structural atomic coordinates of the complex by x-ray diffraction; and
e) modeling the structure of the complex to determine the binding of the candidate compound to the polypeptide.

38. A crystalline preparation of an Fn14 polypeptide (RAG ID NO:6) and a test compound prepared by the method of claim 37.

39. A method of identifying a compound which binds to an Fn14 polypeptide (RAG ID NO:6), the method comprising the steps of:
a) providing a candidate compound;
b) contacting the candidate compound with the Fn14 polypeptide under conditions which a complex is formed between the candidate compound and the Fn14 polypeptide;
c) determining the binding or structure of the complex by methods of nuclear magnetic resonance spectroscopy or mass; and optionally
e) modeling the structure of the complex.

40. A transgenic non-human mammal, having genomically-integrated in non-human mammal cells, comprising a nucleic acid compound having a first segment which is a regulatory region and a second segment which is a polynucleotide sequence encoding an Fn14 polypeptide (RAG ID NO:6), wherein the first segment is operably linked to the second segment.

41. The transgenic non-human mammal of claim 40, wherein the transgenic non-human mammal Is a transgenic mouse.

42. The transgenic non-human mammal of claim 40, wherein the first segment is a regulatable expression element or elements which are subject to cell- or tissue-specific regulation.

43. Tissue or cells derived, or cultured from, the non-human transgenic mammal of claim 40.

44. A transgenic knockout non-human mammal whose genome comprises a homozygous or heterozygous disruption in at least one Fn14 gene (RAG ID NO:6), wherein the homozygous disruption prevents the expression of a functional Fn14 polypeptide (RAG ID NO:6), and wherein the heterozygous disruption reduces the expression of functional Fn14 polypeptide (RAG ID NO:6) in the knockout mouse.

45. The transgenic knockout non-human mammal of claim 44, wherein the transgenic knockout non-human mammal is a transgenic mouse.

46. Tissue or cells derived, or cultured from, the non-human knockout transgenic non-human mammal of claim 44.

47. A method for producing a cell in which the expression of Fn14 gene (RAG ID NO:6) is decreased, wherein the method comprises the steps of: introducing an siRNA expression system into a host cells; and selecting the host cells in which the siRNA expression system is introduced.

48. A cell maintaining an siRNA expression system in which the expression of an Fn14 polypeptide (RAG ID NO:6) encoded by an Fn14 gene (RAG ID NO:6) is decreased.

49. A cell according to claim 48, wherein the cell is a mammalian cell.

50. An antibody or fragment thereof that binds immunospecifically to an Fn14 polypeptide (RAG ID NO:6).

51. An antibody according to claim 50, wherein the antibody is a monoclonal antibody.

52. An antibody according to claim 50, wherein the antibody is a humanized antibody.

53. A method of treating a pathological state in a mammal, the method comprising administering to the mammal the antibody of claim 50 In an amount sufficient to alleviate the pathological state.

54. A pharmaceutical composition comprising an antibody of claim 50 and a pharmaceutically acceptable carrier.

55. A kit comprising in one or more containers, the pharmaceutical composition of claim 50 and instructions for using the contents therein.

56. A method of detecting an Fn14 polypeptide (RAG ID NO:6), the method comprising:
(a) providing a test sample;
(b) contacting the test sample with an anti-Fn14 antibody of claim 50 under conditions which the anti-Fn14 antibody complexes with the Fn14 polypeptide to form an anti-Fn14 antibody/Fn14 polypeptide complexes;
(c) detecting the anti-Fn14 antibody/Fn14 polypeptide complexes; and
(d) quantifying the anti-Fn14 antibody/Fn14 polypeptide complexes in the test sample.

57. A method of identifying a compound that binds to an Fn14 polypeptide (RAG ID NO:6), the method comprising:
(a) contacting the compound with the Fn14 polypeptide; and
(b) determining whether the compound binds to the Fn14 polypeptide (RAG ID NO:6).

58. A method for determining the presence of, or predisposition to, a disease associated with altered levels of an Fn14 polypeptide (RAG ID NO:6) In a first mammalian subject, the method comprising:
(a) providing a test sample from the first mammalian subject;
(b) contacting the test sample from the first mammalian subject with compound that binds the Fn14 polypeptide;
(c) detecting the level of compound/Fn14 polypeptide complex;
(d) quantifying the level of expression of the Fn14 polypeptide in the sample from the first mammalian subject; and
(e) comparing the amount of the Fn14 polypeptide in the sample of step (a) to the amount of the Fn14 polypeptide present in a control sample from a second mammalian subject known not to have, or not to be predisposed to, the disease, wherein an alteration in the expression level of the Fn14 polypeptide in the first subject as compared to the control sample indicates the presence of or predisposition to the disease.

59. A method of regenerating neurons comprising administering to a subject in need thereof an effective amount of a polypeptide encoded by a polynucleotide selected from the group consisting of RAG ID NOs:1-281.

60. A method according to claim 59, wherein a composition that upregulates a polypeptide encoded by a polynucleotide selected from the group consisting of RAG ID NOs:1-281 Is administered to the subject.

61. A method according to claim 59, wherein a composition that down-regulates a polypeptide encoded by a polynucleotide selected from the group consisting of RAG ID NOs:1-281 is administered to the subject.

62. A method according to claim 60, wherein the composition comprises a vector that delivers a polynucleotide selected from the group consisting of RAG ID NOs:1-281.

63. A method according to claim 61, wherein the composition comprises a vector that delivers a polynucleotide selected from the group consisting of RAG ID NOs:1-281.

64. A method according to claim 59, wherein the subject is a human.

65. A method of regenerating neurons comprising administering to a subject in need thereof a RAG compound that modulates the activity of a polypeptide encoded by a polynucleotide selected from the group consisting of RAG ID NOs: 1-281 in amounts effective to cause neuronal regeneration.

66. A method according to claim 65, wherein the subject is a human.

67. A method according to claim 65, wherein the RAG compound increases the activity of a polypeptide encoded by a polynucleotide selected from the group consisting of RAG ID NOs:1-281.

68. A method according to claim 65, wherein the RAG compound increases the expression of a polypeptide encoded by a polynucleotide selected from the group consisting of RAG ID NOs:1-281.

69. A method according to claim 65, wherein the RAG compound decreases the activity of a polypeptide encoded by a polynucleotide selected from the group consisting of RAG ID NOs:1-281.

70. A method according to claim 65, wherein the RAG compound decreases the expression of a polypeptide encoded by a polynucleotide selected from the group consisting of RAG ID NOs:1-281.

71. A method of inhibiting neuron growth comprising administering to a subject in need thereof a RAG compound that modulates the activity of a polypeptide encoded by a polynucleotide selected from the group consisting of RAG ID NOs:1-281 in amounts effective to cause inhibition of neuronal growth.

72. A method according to claim 71, wherein the subject is a human.

73. A method according to claim 71, wherein the RAG compound increases the activity of a polypeptide encoded by a polynucleotide selected from the group consisting of RAG ID NOs: 1-281.

74. A method according to claim 71, wherein the RAG compound increases the expression of a polypeptide encoded by a polynucleotide selected from the group consisting of RAG ID NOs:1-281.

75. A method according to claim 71, wherein the RAG compound decreases the activity of a polypeptide encoded by a polynucleotide selected from the group consisting of RAG ID NOs:1-281.

76. A method according to claim 71, wherein the RAG compound decreases the expression of a polypeptide encoded by a polynucleotide selected from the group consisting of RAG ID NOs:1-281.

77. The use of a compound for the manufacture of a medicament for treatment of a RAG-associated disorder, wherein the compound is selected from the group consisting of a polypeptide encoded by a polynucleotide selected from the RAG ID NOs:1-281.

78. A use according to claim 77, wherein the RAG-associated disorder is selected from the group consisting of brain trauma, spinal cord trauma, stroke, cerebral palsy, multiple sclerosis, Parkinson's disease, Alzheimer's disease, ALS, stroke, peripheral neuropathies, brain cancer, neuroblastoma, and reinnervation of transplanted tissue.

79. A method of treating or preventing a RAG-associated disorder, the method comprising administering to a subject in which such treatment or prevention is desired a polypeptide encoded by a polynucleotide selected from the group consisting of RAG ID NOs:1-281 in an amount sufficient to treat or prevent the RAG associated disorder.

80. A method according to claim 79, wherein the RAG-associated disorder is selected from the group consisting of RAG-associated disorder is selected from the group consisting of: brain trauma, spinal cord trauma, stroke, cerebral palsy, multiple sclerosis, Parkinson's disease, Alzheimer's disease, ALS, stroke, peripheral neuropathies, brain cancer, neuroblastoma, and reinnervation of transplanted tissue.

81. A method according to claim 79, wherein the subject is a human.

82. A method of treating or preventing a RAG-associated disorder, the method comprising administering to a subject in which such treatment or prevention is desired a polynucleotide selected from the group consisting of RAG ID NOs: 1-281 in an amount sufficient to treat or prevent the RAG-associated disorder in the subject.

83. A method according to claim 82, wherein the subject is a human.

84. A method according to claim 82, wherein the RAG-associated disorder is selected from the group consisting of RAG-associated disorder is selected from the group consisting of: brain trauma, spinal cord trauma, stroke, cerebral palsy, multiple sclerosis, Parkinson's disease, Alzheimers disease, ALS, stroke, peripheral neuropathies, brain cancer, neuroblastoma, and reinnervation of transplanted tissue.

85. A method of treating a pathological state in a mammal, the method comprising administering to the mammal a compound in an amount that is sufficient to alleviate the pathological state, wherein the compound is a compound having an amino add sequence at least 90% identical to a compound comprising a polypeptide encoded by a polynucleotide selected from the group consisting of RAG ID NOs:1-281.

86. A method of treating a RAG-associated disorder in a mammal, the method comprising administering to the mammal at least one compound which modulates the expression or activity of a polypeptide encoded by a polynucleotide selected from the group consisting of RAG ID NOs:1-281.

87. A method according to claim 86, wherein the RAG-associated disorder is selected from the group consisting of: brain trauma, spinal cord trauma, stroke, cerebral palsy, multiple sclerosis, Parkinson's disease, Alzheimer's disease, ALS, stroke, peripheral neuropathies, brain cancer, neuroblastoma, and reinnervation of transplanted Tissue.

88. A method of identifying a compound which binds to a polypeptide encoded by a polynucleotide selected from the group consisting of RAG ID NOs:1-281, the method comprising the steps of:
a) providing a candidate compound;
b) contacting the candidate compound with the polypeptide under conditions which a complex is formed between the candidate compound and the polypeptide;
c) Incubating the complex under conditions where co-crystals of the complex form;
d) determining the structural atomic coordinates of the complex by )x-ray diffraction; and
e) modeling the structure of the complex to determine the binding of the candidate compound to the polypeptide.

89. A crystalline preparation of a polypeptide encoded by a polynucleotide selected from the group consisting of RAG ID Nos:1-281 and a test compound prepared by the method of claim 88.

90. A method of Identifying a compound which binds to a polypeptide encoded by a polynucleotide selected from the group consisting of RAG ID NOs:1-281, the method comprising the steps of.
a) providing a candidate compound;
b) contacting the candidate compound with the polypeptide encoded by a polynucleotide selected from the group consisting of RAG ID NOs:1-281 under conditions which a complex is formed between the candidate compound and the polypeptide;
c) determining the binding or structure of the complex by methods of nudear magnetic resonance spectroscopy or mass; and optionally
e) modeling the structure of the complex.

91. A transgenic non-human mammal, having genomically-integrated in non-human mammal cells, comprising a nucleic acid compound having a first segment which is a regulatory region and a second segment which is a polynucleotide sequence encoding a compound selected from the group consisting of RAG ID NOs: 1-281, wherein the first segment is operably linked to the second segment.

92. The transgenic non-human mammal of claim 91, wherein the transgenic non-human mammal is a transgenic mouse.

93. The transgenic non-human mammal of claim 91, wherein the first segment is a regulatable expression element or elements which are subject to cell- or tissue-specific regulation.

94. Tissue or cells derived, or cultured from, the non-human transgenic mammal of claim 91.

95. A transgenic knockout non-human mammal whose genome comprises a homozygous or heterozygous disruption in at least one RAG gene selected from the group consisting of RAG ID NOs:1-281, wherein the homozygous disruption prevents the expression of a functional RAG polypeptide, and wherein the heterozygous disruption reduces the expression of a functional RAG polypeptide in the knockout mouse.

96. The transgenic knockout non-human mammal of claim 95, wherein the transgenic knockout non-human mammal is a transgenic mouse.

97. Tissue or cells derived, or cultured from, the non-human knockout transgenic non-human mammal of claim 95.

98. A method for producing a cell in which the expression of a RAG gene selected from the group consisting of RAG ID NOs:1-281 expression is decreased, wherein the method comprises the steps of: Introducing an siRNA expression system into a host cells; and selecting the host cells in which the siRNA expression system is introduced.

99. A cell maintaining an siRNA expression system in which the expression of a RAG polypeptide encoded by a RAG gene selected from the group consisting of RAG ID NOs:1-281 expression is decreased.

100. A cell according to claim 99, wherein the cell is a mammalian cell.

101. An antibody or fragment thereof that binds immunospecifically to a polypeptide encoded by a polynucleotide selected from the group consisting of RAG ID NOs:1-281.

102. An antibody according to claim 101, wherein the antibody Is a monoclonal antibody.

103. An antibody according to claim 101, wherein the antibody is a humanized antibody.

104. A method of treating a pathological state in a mammal, the method comprising administering to the mammal the antibody of claim 101 In an amount sufficient to alleviate the pathological state.

105. A pharmaceutical composition comprising an antibody of claim 101, and a pharmaceutically acceptable carrier.

106. A kit comprising in one or more containers, the pharmaceutical composition of claim 101 and instructions for using the contents therein.

107. A method of detecting a RAG polypeptide encoded by a polynucleotide selected from the group consisting of RAG ID Nos:1-281, the method comprising:
(a) providing a test sample;
(b) contacting the test sample with an anti-RAG antibody of claim 94 under conditions which the antiRAG antibody complexes with the RAG polypeptide to form an anti-RAG antibody/RAG polypeptide complexes;
(c) detecting the anti-RAG antibody/RAG polypeptide complexes; and
(d) quantifying the anti-RAG antibody/RAG polypeptide complexes in the test sample.

108. A method of identifying a compound that binds to a polypeptide encoded by a polynucleotide selected from the group consisting of RAG ID NOs:1-281, the method comprising:
(a) contacting the compound with the polypeptide encoded by a polynucleotide selected from the group consisting of RAG ID NOs:1-281; and
(b) determining whether the compound binds to the compound of the polypeptide encoded by a polynucleotide selected from the group consisting of RAG ID Nos:1-281.

109. A method for determining the presence of, or predisposition to, a disease associated with altered levels of a RAG polypeptide encoded by a polynucleotide selected from the group consisting of RAG ID NOs:1-281 in a first mammalian subject, the method comprising:
(a) providing a test sample from the first mammalian subject;
(b) contacting the test sample from the first mammalian subject with compound that binds the RAG polypeptide;
(c) detecting the level of compound/RAG polypeptide complex;
(d) quantifying the level of expression of the RAG polypeptide in the sample from the first mammalian subject; and
(e) comparing the amount of the RAG polypeptide in the sample of step (a) to the amount of polypeptide present in a control sample from a second mammalian subject known not to have, or not to be predisposed to, the disease, wherein an alteration in the expression level of the RAG polypeptide in the first subject as compared to the control sample indicates the presence of or predisposition to the disease.
